(19) United States
(12) Patent Application Publication
(10) Pub. No.: US 2021/0318315 A1
(43) Pub. Date: Oct. 14, 2021

(54) ULTRA-HIGH-THROUGHPUT MICROFLUIDIC ENZYME SCREENING PLATFORM FOR ENZYME DEVELOPMENT

(71) Applicant: National University of Singapore, Singapore (SG)

(72) Inventors: Zhi Li, Singapore (SG); Chia-Hung Chen, Singapore (SG); Kang Zhou, Singapore (SG); Jun Li, Singapore (SG); Ji Liu, Singapore (SG); Akbar Vahidi Khalfekandi, Singapore (SG); Guoyun Sun, Singapore (SG); Yuting Wen, Singapore (SG); Kaiyun Tian, Singapore (SG)

(73) Assignee: National University of Singapore, Singapore (SG)

(21) Appl. No.: 17/229,800

(22) Filed: Apr. 13, 2021

Related U.S. Application Data
(60) Provisional application No. 63/199,500, filed on Jan. 4, 2021.
(60) Provisional application No. 63/008,870, filed on Apr. 13, 2020.

(51) Int. Cl. G01N 33/573

(52) U.S. Cl. CPC G01N 33/5735

(57) ABSTRACT

Systems and methods for screening enzyme variants are described, wherein the enzyme variants may catalyze a reaction that is cofactor-dependent or cofactor-independent.

### RELATED APPLICATIONS

This application claims priority under 35 U.S.C. § 119(e) to U.S. Provisional Application No. 63/008,870, filed Apr. 13, 2020, and entitled “Generic Ultra-High-Throughput Microfluidic Enzyme Screening Platform for Enzyme Development,” and to U.S. Provisional Application No. 63/199,500, filed Jan. 4, 2021, and entitled “Generic Ultra-High-Throughput Microfluidic Enzyme Screening Platform For Enzyme Development,” each of which are incorporated herein by reference in their entireties for all purposes.

### TECHNICAL FIELD

Systems and methods for screening enzymes are generally described. The enzymatic activity may depend on the presence of a cofactor or may be independent of the presence of a cofactor.

### BACKGROUND

Biocatalysis, which is the use of enzymes or other biological catalysts to speed up chemical reactions, is a useful tool for green and sustainable synthesis. Compared to chemical catalysis, biocatalysis has the advantages of non-toxicity, mild reaction conditions, high chemo-, regio- and stereo-selectivity, and the ability to perform one-pot multi-step synthesis via cascade reactions. Therefore, biocatalysis has become a key area of focus for pharmaceutical, food and beverages, cosmetics, fragrance and nutraceuticals industries, chemical and biochemical manufacturing, and synthetic biology.

For the successful application of biocatalysis in various industries, it is important to identify enzymes with high activity and desired selectivity and stability for the target application. Although advances in functional genomics have allowed the discovery of numerous new enzymes from various sources, most naturally-occurring enzymes are not optimized for practical applications due to differences between the cellular environment and the industrial setting. Therefore, approaches such as directed evolution, which involves the generation of enzyme variants via random or targeted mutagenesis and the subsequent screening of enzyme variants, have been used to fine-tune enzymes for practical applications.

The traditional microtiter plate-based enzyme screening approach suffers from low throughput and prolonged development times. The use of microfluidic-based platforms has enabled high-throughput enzyme screening to speed up enzyme development via directed evolution. However, the majority of current state-of-the-art microfluidics-based technologies rely on labeled reaction substrates, for example fluorophore-labeled substrates, for the generation of the fluorescence signal in the enzyme screening process. Accordingly, improved systems and methods are needed.

### SUMMARY

Systems and methods for screening enzymes are generally described. The enzymatic activity may depend on the presence of a cofactor or may be independent of the presence of the cofactor. The subject matter of the present disclosure involves, in some cases, interrelated products, alternative solutions to a particular problem, and/or a plurality of different uses of one or more systems and/or articles.

In one aspect, a method of screening an enzyme for one or more characteristics is described, the method comprising the steps of: (a) generating a plurality of droplets, wherein at least one droplet comprises: an enzyme, a reaction substrate; and one or more redox cofactors, or one or more redox cofactors and one or more detection reagents, wherein the enzyme converts the reaction substrate to a target product; (b) detecting a signal emitted from the droplet, wherein the signal is a fluorescence signal, a bioluminescence signal or a chemiluminescence signal, (i) wherein the signal is emitted when the redox cofactor is oxidized or reduced when the reaction substrate is converted to the target product by the enzyme, or (ii) wherein the signal is emitted when the redox cofactor is oxidized or reduced when the target product is further converted to a subsequent product by the one or more detection reagents in the droplet.

In another aspect, a method of screening an enzyme for one or more characteristics is described, the method comprising, in a fluidic device comprising an incubation region comprising a plurality of incubation chambers including a first incubation chamber and a second incubation chamber, wherein the plurality of incubation chambers are configured to allow continuous flow of a plurality of droplets, performing the steps of: culturing a cell in the droplet to form a plurality of cells while flowing the cell from the first incubation chamber to the second incubation chamber; continuously flowing the droplet in the incubation region, wherein the droplet comprises, an enzyme, a reaction substrate, and one or more redox cofactors, or one or more redox cofactors and one or more detection reagents, wherein the enzyme converts the reaction substrate to a target product; and generating a signal from the droplet, wherein the signal is a fluorescence signal, a bioluminescence signal or a chemiluminescence signal.

In another aspect, a fluidic system, comprising, the fluidic system comprising a plurality of droplets including a first droplet comprising a redox cofactor, wherein a concentration of the redox cofactor in the droplet is greater than or equal to 5 μM; a microfluidic channel containing the droplet; and a droplet-sorting region in fluidic communication with the microfluidic channel, wherein the droplet-sorting region comprises a set of electrodes adjacent to the microfluidic channel.

In another aspect, a fluidic system is described, the fluidic system comprising a microfluidic channel containing a plurality of droplets, wherein at least one droplet comprises a redox cofactor and a detection reagent; and wherein the detection reagent is configured to react with a redox couple of the redox cofactor to produce a luminescent signal.

In yet another aspect, a fluidic system is described, the fluidic system comprising, a first reagent chamber configured to contain a first liquid and a plurality of cells, at least one of the cells comprising an enzyme; a second reagent chamber, wherein the second chamber is configured to contain a reaction substrate; a carrier fluid chamber configured to contain a second liquid immiscible with the first fluid, and in fluidic communication with at least the first reagent chamber; a merging region configured to allow merging of the first and second liquids; an incubation region in fluidic communication with the merging region, wherein the incubation region comprises: a plurality of incubation chambers including a first incubation chamber and a second incubation chamber in fluidic wherein the first and second incubation chambers are configured to allow continuous flow of droplets between the chambers; a droplet-sorting region in fluidic communication with the incubation region, wherein the droplet-sorting region comprises: a detection area, a set of electrodes downstream from the detection area configured to sort the plurality of droplets based on detection of a component in the droplet, a collection channel, and a waste channel; and a bubble trap positioned between the incubation region and the droplet-sorting region. In some embodiments, the fluidic system of the preceding claim, further comprising a second carrier fluid chamber within the droplet-sorting region.

In another aspect still, a fluidic system is described, the fluidic system comprising a first reagent chamber configured to contain a first liquid and a plurality of cells, at least one of the cells comprising an enzyme; a second reagent chamber, wherein the second chamber is configured to contain a reaction substrate; a carrier fluid chamber configured to contain a second liquid immiscible with the first fluid, and in fluidic communication with at least the first reagent chamber; a merging region configured to allow merging of the first and second liquids; an incubation region in fluidic communication with the merging region, wherein the incubation region comprises a plurality of incubation chambers including a first incubation chamber and a second incubation chamber; a detection area comprising a first detector, wherein the first detector is configured to determine a first luminescent signal of a first wavelength and a second luminescent signal of a second wavelength, wherein the first wavelength and the second wavelength are different; a droplet-sorting region in fluidic communication with the incubation region; a set of electrodes downstream from the detection area configured to sort the plurality of droplets based on detection of a component or components in the droplet; a collection channel; and a waste channel.

In another aspect, there is provided a method of screening an enzyme for one or more characteristics, the method comprising the steps of: (a) generating a plurality of droplets, wherein at least one or more of the plurality of droplets comprises: an enzyme, a reaction substrate; and one or more redox cofactors, or one or more redox cofactors and one or more detection enzymes, wherein the enzyme converts the reaction substrate to a target product; (b) detecting a signal emitted from each droplet, wherein the signal is a fluorescence signal, a bioluminescence signal or a chemiluminescence signal, and (i) wherein the signal is emitted when the one or more redox cofactors is oxidized or reduced when the reaction substrate is converted to the target product by the enzyme; or (ii) wherein the signal is emitted when the one or more redox cofactors is oxidized or reduced when the target product is further converted to a subsequent product by the one or more detection enzymes in the droplet; (c) screening the enzyme for the one or more characteristics based on the level of emitted signal detected compared to a reference signal, wherein a change in the level of emitted signal compared to the reference signal indicates that the enzyme has the one or more characteristics; and (d) isolating the droplets based on the level of emitted signal detected compared to the reference signal.

In another aspect, provided herein is a use of the method of any one of the preceding claims for screening an enzyme for increasing conversion of its natural substrate, increasing conversion of its non-natural substrate, increasing catalytic activity, increasing stereoselectivity, increasing thermal stability, increasing stability in a predetermined pH range, or a combination thereof.

Other advantages and novel features of the present disclosure will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B show cross-sectional schematic side views of a droplet containing a cell containing a target enzyme merging with reagents, according to some embodiments;

FIGS. 1C-1E show cross-sectional schematic side views illustrating the generation of a luminescent signal in a screening using a detection reagent, according to some embodiments;

FIG. 1F shows a cross-sectional schematic side view illustrating the generation of a luminescent signal in a droplet screening without the use of a detection reagent, according to some embodiments;

FIG. 2A shows a schematic illustration of a microfluidic device for screening enzymes, according to some embodiments;

FIG. 2B shows a microfluidic device on chip for screening enzymes, according to some embodiments;

FIGS. 3A-3B show schematic side views of a portion of a droplet-sorting region in an OFF position in which the droplets are not sorted and in an ON positioned in which the droplets are sorted, according to some embodiments;

FIG. 4 shows a schematic illustration of the ultrahigh-throughput microfluidic screening systems for directed evolution of redox cofactor-dependent or independent enzymes. The oxidized or reduced cofactor that produced from the target biotransformation is detected directly, using red fluorescence reagents, bioluminescence, quantum dots, or biosensor assays or produced and detected through enzymatic cascade reactions by conversion of the target biotransformation product. These systems include: a) Intracellular enzyme with whole-cell and detection of cofactor dependent or independent enzymes via conversion of the target biotransformation product with another enzymatic reaction in cascade to produce oxidized or reduced cofactor. b) Surface displayed cofactor-independent enzyme with the reaction detection via conversion of the target biotransformation product with another enzymatic reaction in cascade to produce oxidized or reduced cofactor. c) Intracellular cofactor-independent enzyme with lysed cell and the reaction detection via conversion of the target biotransformation product with another enzymatic reaction in cascade to produce oxidized or reduced cofactor. d) Surface displayed cofactor-dependent enzyme with the detection of oxidized or reduced cofactor. e) Intracellular cofactor-dependent enzyme with lysed cell and the detection of NAD(P)H or NAD(P)⁺, according to some embodiments;

FIG. 5 shows a schematic illustration of the ultrahigh-throughput microfluidic screening systems for directed evolution of both redox cofactor-dependent or independent enzymes with low activity and or low expression level. The oxidized or reduced cofactor that produced from the target biotransformation is detected directly, using red fluorescence reagents, bioluminescence, quantum dots, or biosensors assays or produced and detected through enzymatic cascade reactions by conversion of the target biotransformation product. These systems undergo additional steps of cell growth and droplet-merging and include, a) Intracellular enzyme with whole-cell and detection of cofactor-dependent or independent enzymes via conversion of the target biotransformation product with another enzymatic reaction in cascade to produce oxidized or reduced cofactor. b) Extracellular cofactor-independent enzymes with the reaction detection via conversion of the target biotransformation product with another enzymatic reaction in cascade to produce oxidized or reduced cofactor. c) Intracellular cofactor-independent enzyme with lysed cell and the reaction detection via conversion of the target biotransformation product with another enzymatic reaction in cascade to produce oxidized or reduced cofactor. d) Extracellular cofactor-dependent enzyme with the detection of oxidized or reduced cofactor. e) Intracellular cofactor-dependent enzyme with lysed cell and the detection of oxidized or reduced cofactor, according to some embodiments;

FIGS. 6A-6E show a calibration curve of the direct NADH fluorescence detection signal at 450 nm with NADH concentration in FIG. 6A) microtiter plate, and FIG. 6B) microfluidic droplets with diameter of 30 μm. Calibration curve of the resazurin-based fluorescence signal from NADH at 590 nm with NADH concentration in FIG. 6C) microtiter plate, and FIG. 6D) microfluidic droplets with diameter of 30 μm. FIG. 6E) Reaction scheme for the fluorescence detection of NAD(P)H with resazurin, according to some embodiments;

FIG. 7 shows fluorescence (450-490 nm) images of droplets with different NADH concentrations. Detection was performed with a complementary metal oxide semiconductor (CMOS) camera with the exposure time of 200 ms. Droplets were kept in a polydimethylsiloxane (PDMS) observing chip with an excitation at 350 nm, according to some embodiments;

FIGS. 8A-8C show FIG. 8A) reaction scheme for the bioluminescence detection of NADH. The calibration curve of the bioluminescence signal at different NADH concentration in FIG. 8B) microtiter plate, and FIG. 8C) microfluidic droplets with diameter of 30 μm, according to some embodiments;

FIG. 9 shows the bioluminescent detection of droplets containing butanol dehydrogenase (BDHA), 1-phenyl-1,2-ethanediol (100 μM) and NAD+ in potassium phosphate (KP) buffer (pH 7.5, 50 mM) obtained from photomultiplier tube (PMT). Data are plotted based on PMT signal that obtained from the flow of the droplets containing reaction mixture and bioluminescent kit reagent, according to some embodiments;

FIGS. 10A-10B show FIG. 10A) structure of quantum dot-based NADH sensor. CdSe-core CdS(2Ls)/Cd0.5Zn0.5S(3L)/ZnS(2Ls) multi shell quantum dots were synthesized and solubilized by 3-mercaptopropionic acid. The water -soluble quantum dots are further modified with bovine serum albumin (BSA) and Nile blue. FIG. 10B) Working principle of NADH quantum dot-based sensor. The sensor is efficiently quenched by the Nile blue through FRET. The Nile blue dye assisted the oxidation of the NAD(P)H cofactors. In the presence of NADH, the dye is reduced to a different form that was not able to absorb photons in the visible spectrum, thereby stopping the quenching and recovers the fluorescence, according to some embodiments;

FIGS. 11A-11C show NADH sensing on microfluidic system. PMT signals were recorded. FIG. 11A) PMT signals of quenched QDs-Nile blue. FIG. 11B) PMT signals of fluorescent QDs-BSA. FIG. 11C) PMT signals of QDs treated with 500 mM NADH, PMT signals of 1000 mM NADH was recorded as control, according to some embodiments;

FIGS. 12A-12B show FIG. 12A) working principle of the NAD⁺ biosensor. FIG. 12B) Demonstrating the NAD⁺ detection with biosensor in microfluidics system, according to some embodiments;

FIGS. 13A-13B show FIG. 13A) synthesis scheme of NAD⁺ quantum dot sensor, CdSe-core CdS(2Ls)/Cd_{0.5}Zn_{0.5}S(3 L)/ZnS(2 Ls) multi shell quantum dots. FIG. 13B) Working principle of NAD⁺ quantum dot-based sensor, according to some embodiments;

FIGS. 14A-14B show the NAD⁺ detection quantum dot sensor in microfluidics. FIG. 14A) Droplet signals obtained from Qd sensor mixing with various concentration of NAD⁺, detected by photomultiplier tube (PMT). FIG. 14B) Fluorescent microscopic images of mixing droplets, scale bar=100 μm, according to some embodiments;

FIG. 15 shows the PMT signals of fluorescent droplets. The sampling rate could reach to 300 kHz, according to some embodiments;

FIG. 16 shows a schematic representation of the microfluidic sorting chip design, according to some embodiments;

FIGS. 17A-17F show the sorting performance of the droplet sorting chip. Sorting sample consists of 10% positive and 90% empty (or blank) droplets containing red fluorescence. FIG. 17A) Droplets flow to waste channel when the pulse trigger is OFF. FIG. 17B) Droplets flow to collect channel when pulse trigger is ON. FIG. 17C) Droplets collected from waste channel after sorting. FIG. 17D) Droplets collected from collector channel after sorting. The scale bar is 50 μm. The fluorescence images of after sorting of NAD(P)H using its direct fluorescence detection at 450 nm, FIG. 17E) droplets in the collector, and FIG. 17F) droplets in the waste channel, according to some embodiments;

FIG. 18 shows images of fluorescence droplets containing E. coli expressing secondary alcohol dehydrogenase from Candida parapsilosis (CpSADH) in the collector and waste channel after sorting, according to some embodiments;

FIGS. 19A-19B show FIG. 19A) schematic diagram of the operation of a pico-injection device. FIG. 19B) The performance of the device for droplet merging as determined with two orthogonal fluorescence signals, according to some embodiments;

FIG. 20A shows a time course of the oxidation of racemic 2-octanol (1 mM) with the lysed cells of different variants, according to some embodiments;

FIG. 20B shows the reaction course of the oxidation of (R)-2-octanol with purified wild type CpSADH or mutants. Reaction was performed in 1 mL of phosphate buffer (100 mM, pH 8.0) containing purified CpSADH or the mutants (1.5 μM), (R)-2-octanol (1 mM), and NAD⁺ (2 mM) at 24° C. NADH concentration was determined with a UV spectrophotometer at 340 nm, according to some embodiments;

FIG. 21 shows conversion of the oxidation of rac-2-octanol (10 mM) to ketone 2a with E. coli cells (5 g cdw/L) expressing CpSADH or the mutants in the presence of acetone (2% v/v) for NAD⁺ regeneration in 5 mL reaction volume at 24° C. for 4 h and 24 h. The concentrations of 2-octanol and 2-octanone were determined by GC analysis, according to some embodiments;

FIG. 22 shows a schematic illustration of activity assay of epoxide hydrolase (EH) as a cofactor-independent enzyme through NADH production and detection via cascade reactions, according to some embodiments;

FIGS. 23A-23B show NADH generated from FIG. 23A) cascade hydrolysis and oxidation of cyclohexene epoxide in droplets and FIG. 23B) cascade hydrolysis and oxidation of styrene oxide in droplets, according to some embodiments;

FIG. 24 shows a schematic illustration of activity assay of styrene oxide isomerase (SOI) for isomerization of styrene oxide through single-cell encapsulation and NADH production via cascade reaction, according to some embodiments;

FIGS. 25A-25B show a schematic illustration of FIG. 25A) NADH-dependent activity assay of an alcohol dehydrogenase (CpsADH) for the oxidation of 2-octanol through single-cell encapsulation and surface displayed enzyme, and FIG. 25B) plasmid construction of surface displayed system of CpsADH in E. coli cells, according to some embodiments;

FIGS. 26A-26B show enzyme screening through cell growth strategy. FIG. 26A) Schematic illustration of P450 monooxygenase (P450pyr) catalyzed hydroxylation of alkane and detection via NADH produced from cascade reaction. FIG. 26B) Number of cells required to achieve detectable reaction conversion, according to some embodiments; and

FIGS. 27A-27B show FIG. 27A) the growth of encapsulated E. coli (P450pyr) cells in droplets. FIG. 27B) The cell counts in droplet at different growth conditions, according to some embodiments.

### DETAILED DESCRIPTION

Systems and methods for screening enzymes are described herein. These systems and methods may be used to screen a plurality of enzyme variants, each contained in a cell within a droplet. While some microfluidic systems for screening enzymes exist, these systems suffer from significant drawbacks. For example, certain existing microfluidics-based screening platforms use labeled substrates or colorimetric detection of a cofactor, such as NADH, in order to screen enzymes. However, this approach suffers from low sensitivity and requires the pre-addition of a high concentration of NADH, and results in low accuracy in identifying a positive enzyme variant. Such an existing system requires the enzyme involve the cofactor. In addition, the throughput of these existing screening processes can be low and may also be based on the absorbance of a particular droplet. This approach also requires the enzyme produce NADH to produce a colorimetric signal but is ineffective for enzymatic reactions that do not produce NADH.

By contrast, the systems (e.g., fluidic systems, microfluidic devices) and methods disclosed herein may screen an enzymatic reaction that is dependent or independent on a cofactor (e.g., NADH). When screening a cofactor-independent enzyme, a detection reagent, such as a detection enzyme, may be configured to react with the target product (e.g., in a cofactor-dependent reaction) in order to produce a signal (e.g., a luminescent signal, a fluorescent signal). In such a scenario, the enzymatic reaction may not depend on or involve the cofactor, while the detection reagent is configured to involve the cofactor. For example, in some embodiments, the detection reagent is a detection enzyme that reacts with a target product to produce a subsequent product and may also react or interact with the cofactor (e.g., via a redox reaction) to generate a redox couple of the cofactor. The subsequent product may be configured to generate a luminescent signal in order to screen an enzymatic reaction of a target enzyme within a droplet (or otherwise detect, sort, and/or isolate a droplet generated the luminescent signal). In this way, the detection enzyme indirectly links the activity of the enzymatic reaction of the target enzyme to a luminescent signal generated by the detection enzyme and the subsequent product. This luminescent signal (or two or more luminescent signals) generated within a droplet containing the target enzyme (and detection enzyme) may be used to sort this droplet of interest from undesired droplets not containing the desired enzymatic activity or that do not contain the target enzyme (or variant of the target enzyme, i.e., blank droplets).

The signal may be a luminescent signal, such as fluorescence signal, a bioluminescence signal or a chemiluminescence signal. Advantageously, the use of a luminescent signal for screening or detecting avoids the drawbacks of colorimetric or absorbance-based detection methods, such as a low sensitivity. That is to say, luminescent-based detecting and screening is often more sensitive than colorimetric detection and hence the system and methods described herein may be more sensitive or selective for screening or detecting droplets that contain enzyme variants of interest compared to certain existing enzyme screening systems and devices. In some embodiments, two or more distinct luminescent signals (e.g., a first luminescent signal, a second luminescent signal) may be generated within the same (or different) droplet, and each luminescent signal may provide distinct information about a droplet (or other component of the system, such as a fluid), such as the number of cells within a droplet and/or information related to a cofactor (e.g., cofactor concentration, enzymatic activity).

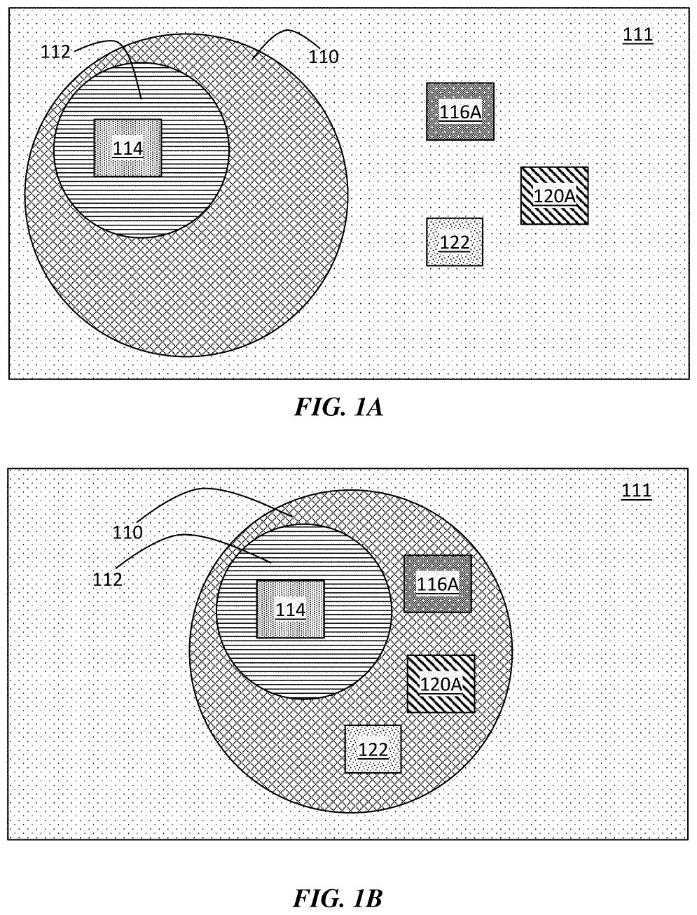

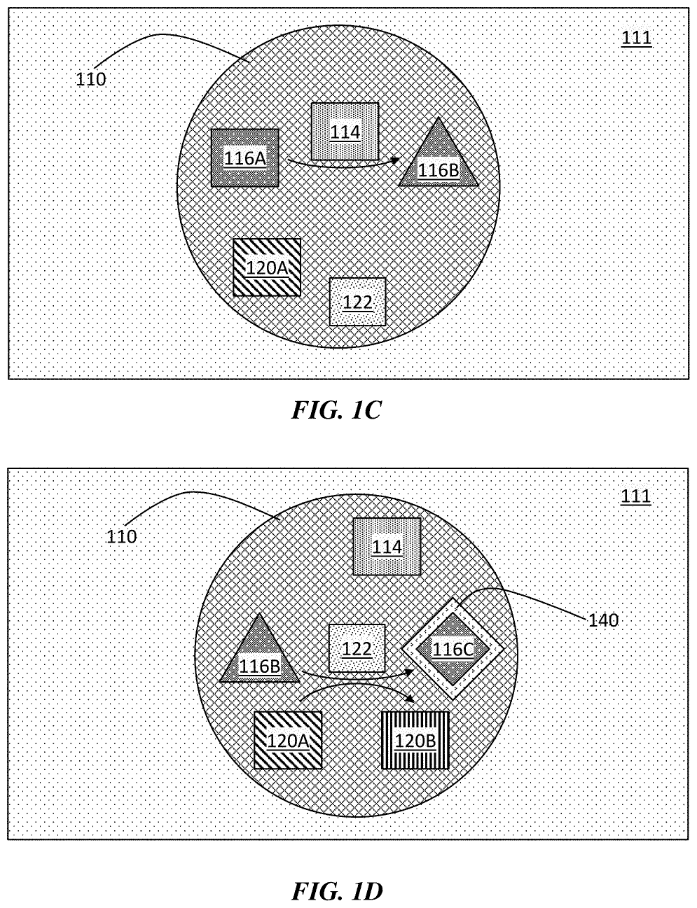

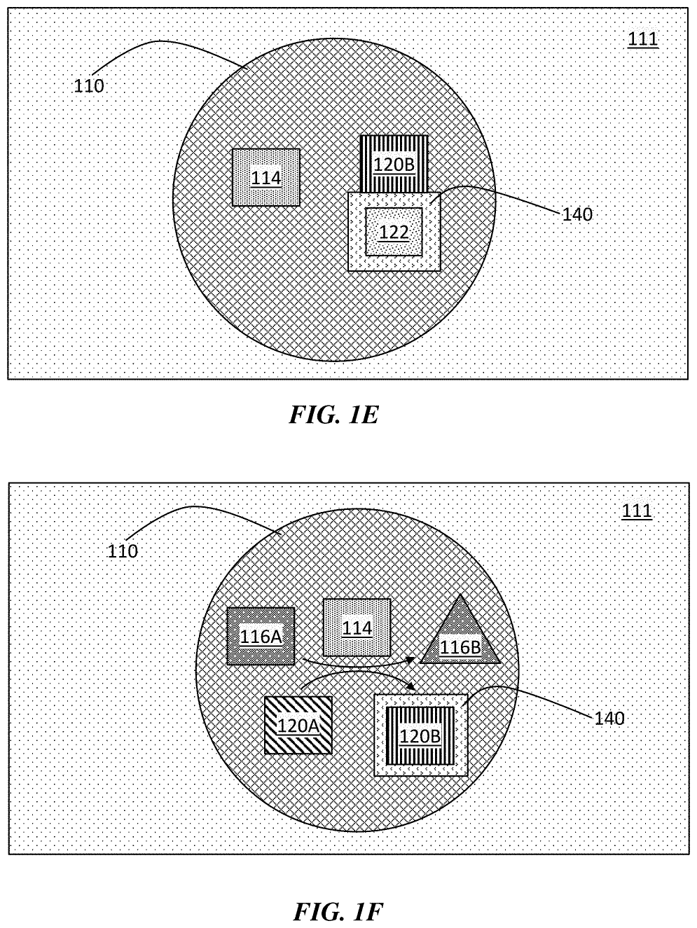

FIGS. 1A-1E provide schematic illustrations showing the generation of a luminescent signal by an enzyme (e.g., a target enzyme, an enzyme variant) and one or more detection reagents. In FIG. 1A, a droplet 110 is disposed within a microfluidic channel 111 comprising a single cell 112, and The single cell 112 comprising a target enzyme 114 to be screened for a particular property (e.g., one or more characteristics as described elsewhere herein). Also disposed within the microfluidic channel 111 are a reaction substrate 116A, a cofactor 120A, and a detection reagent 122. These reagents may be merged (e.g., within a merging region) into the droplet. For example, in FIG. 1B, the droplet 110 contains the reaction substrate 116A, the cofactor 120A, and the detection reagent 122, along with cell 112. In some embodiments, a set of merging electrodes may facilitate the introduction and/or merging of the one or more reagents into the droplet (not pictured).

To facilitate the generation of a luminescent signal, in some embodiments, the cells may be lysed within a droplet (e.g., using a lysis buffer) so that the components of the cell (including the target enzyme) may react or interact with the reagents added to the droplet. Lysis of the cell within the droplet may occur before or after introduction of additional reagents into the droplet and may also occur prior to incubation or after incubation of the cell. In some embodiments, additional reagents are introduced into the droplet before and/or after cell lysis. However, it should be understood that, in some embodiments, one or more reagents may be capable of diffusing into the cell (via its cell membrane) such that lysis of the cell is not always required. Additionally, in some embodiments, a target enzyme may be displayed on the surface of the cell such that lysis is not required for the target enzyme to react or interact with the reagents or additional reagents.

In FIG. 1C, the cell 112 has been lysed such that the target enzyme 114 may interact with the reaction substrate 116A. The target enzyme 114 is configured to catalyze the conversion of reaction substrate 116A into target product 116B. Advantageously, encapsulating the cell within the droplet may prevent undesired merging of the contents of the cell from other cells containing enzymes for screening (e.g., merging or fusing of cells). In this manner, each droplet may act as an environment for an individual target enzyme or enzyme variant to be expressed without undesired influence from other target enzymes or enzyme variants contained in other droplets (e.g., other droplets containing other cells with other target enzymes). It should be noted, however, that while FIGS. 1A-1B show encapsulation of a single cell within the droplet, a plurality of cells may be encapsulated within the droplet (not shown). In such an embodiment where the droplet contains a plurality of cells, each target enzyme (directly or indirectly) within each cell may generate a luminescent signal. The plurality of cells may be encapsulated within a droplet or a single cell may be encapsulated in a droplet and proliferate (e.g., multiply) into a plurality of cells, each cell containing the target enzyme. In some such embodiments, the cells may be lysed, such the luminescent signal of the droplet may be an average (e.g., a cell-normalized average) or accumulation of the luminescent signal of each target enzyme of each cell that was within the droplet (also not shown).

The target enzyme may convert the reaction substrate into a target product in a cofactor-independent reaction. For example, in FIG. 1C, target enzyme 114 catalyzes the conversion of reaction substrate 116A into target product 116B. In some embodiments, he enzymatic reaction of target enzyme 114 does not involve or depend on an interaction with the cofactor 120A. Where target product 116B does not directly generate a luminescent signal, one or more detection reagents, such as detection reagent 122, may be configured to utilize target product 116B, in one or more reactions, to generate a luminescent signal.

In some embodiments, the detection reagent is configured to react (or catalyze a reaction) with a target product of the target enzyme and may also be configured to react or interact with one or more cofactors. That is, in some such embodiments, while the target enzyme may catalyze a reaction that does not directly involve the cofactor, the detection reagent may be configured to directly react or interact with the cofactor. In this manner, the systems and methods disclosed may be used to screen a target enzyme that does not directly involve the cofactor (i.e., the target enzyme catalyzes a reaction that is independent of the presence of the cofactor). For example, FIG. 1D schematically shows the conversion of target product 116B by detection reagent 122 (e.g., a detection enzyme) and cofactor 120A into a luminescent product 116C, which generates a luminescent signal 140. Cofactor 120A is concomitantly oxidized or reduced into redox couple 120B when detection reagent 122 reacts with target product 116B. Redox couple 120B is related to cofactor 120A in that the two are redox pairs of one another (i.e., related to one another via one or more oxidation-reduction reactions).

In some embodiments, a redox couple of the cofactor may react or interact with a detection reagent. For example, FIG. 1E schematically illustrates an embodiment where redox couple 120B reacts with the detection reagent 122 to generate a luminescent signal 140 from the detection reagent 122. For example, the detection reagent 122 may be a quantum dot functionalized with a ligand that may interact with the cofactor to generate (or quench) a fluorescent signal. It should be appreciated that, in some embodiments, more than one detection reagents may be present in the droplet 110, such that the redox couple 120B shown in FIG. 1E may be produced by a detection enzyme (e.g., as shown in FIG. 1D). In some embodiments, the redox couple 120B shown in FIG. 1E may be produced by a target enzyme (e.g., as shown in FIG. 1F). As discussed in more detail below, luminescent signal 140 may be used to screen, detect, and/or isolate droplets containing the target enzyme(s) of interest.

In some embodiments, screening, detecting, and/or isolating an enzyme (e.g., a target enzyme, an enzyme variant) involves an enzymatic reaction that does not require a detection reagent to generate the luminescent signal. That is, the target enzyme may directly generate a fluorescent signal upon oxidization or reduction of the cofactor. By way of illustration, in FIG. 1F, the target enzyme 114 catalyzes the conversion of the reaction substrate 116A into target product 116B with concomitant oxidation or reduction of the cofactor 120A to form redox couple 120B, which is configured to emit luminescent signal 140. In An exemplary embodiment, the cofactor is NAD+ and the redox couple is NADH, which may generate a luminescent signal (e.g., a fluorescent signal) upon reduction from NAD⁺ to NADH.

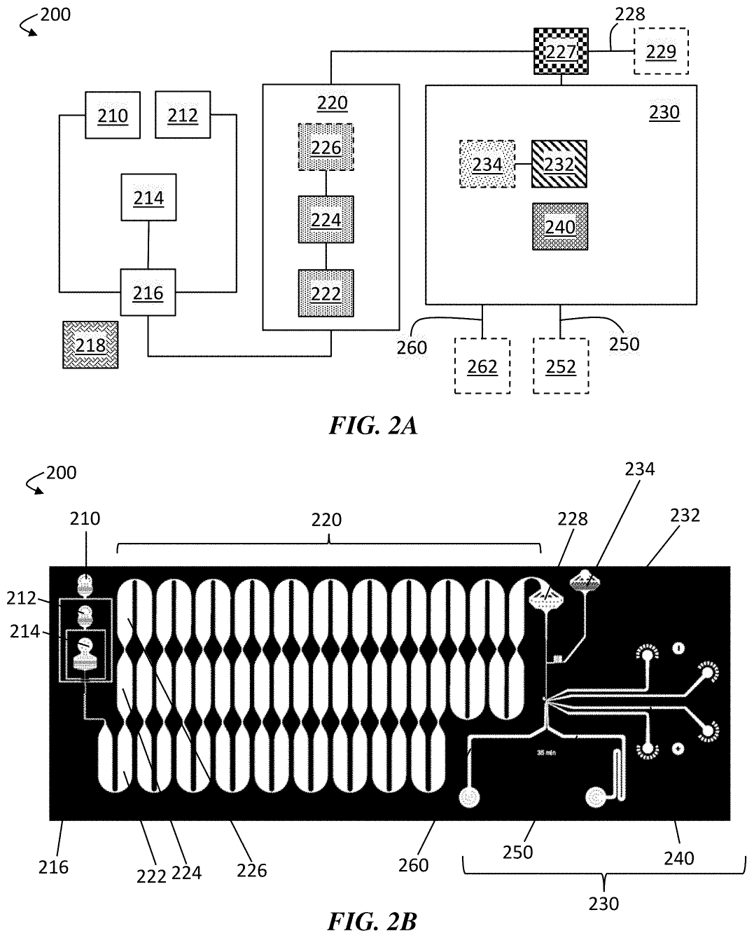

FIG. 2 shows a schematic illustration of a system for screening enzymes (e.g., target enzymes, enzyme variants) within cells contained in droplets. Microfluidic device 200 comprises a first reagent chamber 210 and a second reagent chamber 212. The first reagent chamber may comprise or be configured to contain cells (e.g., single cells) where each cell comprises a target enzyme or an enzyme variant of the target enzyme. In some embodiments, the first reagent chamber may also comprise other reagents, such as surfactants, buffers, cofactors, and/or reagents for culturing and/or proliferating the cells. The second reagent chamber may comprise or be configured to contain a reaction substrate, where each target enzyme or enzyme variant is configured to catalyze the enzymatic reaction of the reaction substrate to a target product. In some embodiments, the second reagent chamber may also comprise other reagents, such as surfactants, buffers, cofactors, and/or reagents for culturing and/or proliferating cells.

The microfluidic device 200 may also comprise a carrier fluid chamber 214. The carrier fluid chamber may comprise or be configured to contain a carrier fluid, such as an oil. In some embodiments, the carrier fluid is selected so that it is immiscible with reagents (or a liquid containing the reagents) of the first reagent chamber and/or the second reagent chamber. For example, if the first reagent chamber or the second reagent chamber contains aqueous solution or aqueous-based reagents, the carrier fluid chamber may contain an oil immiscible with water. Non-limiting examples of a carrier fluid include oils, such as hydrocarbon oil, mineral oil, fluorinated oil, and/or silicone oil. Other carrier fluids are possible. In other embodiments, the carrier fluid may be miscible with some or all of the reagents or a liquid containing some or all of the reagents as this disclosure is so limited.

As described above and elsewhere herein, each cell containing an enzyme may be encapsulated into a droplet, and the droplet may also contain other reagents (e.g., cofactors, culturing reagents) to facilitate conversion of the reaction substrate into the target product and for detection of the target product (e.g., via one or more detection reagents). The droplet may be formed at or within the merging region (e.g., where the reagent fluid(s) of the first reagent chamber and/or the second reagent chamber are contacted with the carrier fluid).

In some embodiments, a set of electrodes is positioned adjacent to the merging region to facilitate merging of reagents (e.g., detection reagents) into the droplet. For example, in FIG. 2A, merging electrodes 218 are adjacent to the merging region 216. The merging electrodes may facilitate control over the volume of the reagents merged within the droplet by controlling the potential applied by the electrodes relative to the droplet and/or the reagents.

In other embodiments, droplets may be generated off-chip and then subsequently introduced into the system or method.

A plurality of droplets, at least some of which contain a cell with a target enzyme, may flow into an incubation region to provide time for the target enzyme to catalyze the reaction of the reaction substrate. For example, in FIG. 2A, the incubation region 220 comprises a plurality of incubation chambers including a first incubation chamber 222 and a second incubation chamber 224. As shown in the figure, the chambers of the incubation region may be joined by a channel with a smaller maximum cross-sectional transverse dimension, followed by an adjacent chamber with a larger maximum cross-sectional transverse dimension as the channel moves towards the larger area of the adjacent chamber. Such a configuration can be used to adjust the time provided for cell incubation within each droplet. Such a configuration (e.g., a channel having a relatively large maximum cross-sectional transverse dimension directly adjacent to a channel having a relatively small maximum cross-sectional transverse dimension) had been avoided in certain existing microfluidic systems, as it was believed it could result in the formation of gas bubbles or negatively affect the flow rate and/or result in clogging of the device, but it has been recognized and appreciated in the context of the present disclosure that such a configuration can be used to adequately incubate cells within the droplets, and, in some cases tune incubation time of the droplets within the incubation region. In some cases, cells may be cultured and/or proliferated (e.g., grown) within the incubation region. In some embodiments, cells may be cultured and/or proliferated during continuous flow of fluid and/or the droplets in the incubation region and/or the device. Advantageously, the configuration of the incubation region described herein can also provide for screening of the droplets in relative sequential order. For example, if a plurality of droplets (at least some of which contain a target enzyme or enzyme variant) enters the incubation region in a particular order, that order may be at least partially preserved when the plurality of droplets exits the incubation region. The incubation region may further comprise additional incubation chambers (e.g., a third incubation chamber, a fourth incubation chamber, a fifth incubation chamber, and so forth), such as additional incubation chamber 226 shown in FIG. 2A. Those skilled in the art in view of the teachings of the present disclosure will be capable of choosing the appropriate number and dimensions of the incubation chambers within the incubation region based on a desired incubation time and/or culturing step.

In some embodiments, a flow containing a plurality of droplets may pass through a bubble trap, for example, after exiting the incubation region. In some such embodiments, the bubble trap may advantageously remove and/or reduce the size of any air bubbles formed during the flow or incubation of the cells in the incubation region (or an incubation chamber). By way of illustration, in FIG. 2A, a bubble trap 227 is positioned adjacent to and downstream of the incubation region. In such a configuration, the bubble trap 227 may remove gas bubbles from a stream as it flows from the incubation region 220 to a droplet -sorting region 230. Optionally, bubble trap 227 may comprise a vacuum port 228 for attaching to a vacuum 229. The vacuum may be used to place the bubble trap under reduced pressure to facilitate the removal of gas from a flow or a stream within the system 200. In some embodiments, the bubble trap comprises a membrane, indentations, protrusions, a surfactant, or any other suitable feature that can remove and/or reduce the size of air bubbles in the channel. The bubble trap may, for example, be position in, on, and/or adjacent to the channel.

The droplet-sorting region may be used to separate droplets that contain target enzymes at or above an adequate threshold (i.e., droplets of interests, droplets containing enzymes that have one or more characteristics) from undesired droplets or droplets that do not express the target enzyme, do not express the target enzyme at an adequate threshold (e.g., enzymes that do not have one or more characteristics), and/or do not contain any cells (e.g., blank droplets). For example, in FIG. 2A, the droplet -sorting region 230 is adjacent to and downstream of the incubation region 220 and bubble trap 227. After incubation, one or more droplets containing one or more cells each with a target enzyme may enter the droplet -sorting region where they may be screened and sorted accordingly. The droplet -sorting region includes a detection area 232 for detecting a luminescent signal (e.g., a fluorescent signal) within each droplet of the plurality of droplets that passes through the detection area. The droplet-sorting region also includes a set of sorting electrodes 240 for attracting or urging droplets of interest towards a collection channel 250, as determined by luminescent signal (or ratio of luminescent signals) produced by the droplet containing the target enzyme. The detection area 232 may be in electronic communication with a set of sorting electrodes 240 so as to provide a signal to sort (or not sort) the droplets. The detection area 232 may also be in electronic communication with a controller (e.g., a microcontroller) and/or one or more detectors located at an upstream or downstream position. In some embodiments, the detection area includes two or more detectors, including a first detector and a second detector. In some cases, the first detector is positioned upstream of the sorting electrodes and a second detector positioned downstream of the sorting electrodes. In other embodiments, both detectors are positioned upstream of the sorting electrodes. Other configurations are also possible.

In some embodiments, the sorting region 230 may optionally comprise a spacing fluid chamber comprising or configured to contain a spacing fluid. The spacing fluid chamber may merge with the channel containing the droplets at an intersection. Advantageously, the spacing fluid chamber may provide or introduce a spacing fluid for spacing or separating incoming droplets, which may facilitate or improve the detection of the luminescent signal or the sorting of the droplets. The spacing fluid may be the same or different from the carrier fluid within the carrier fluid chamber, if present.

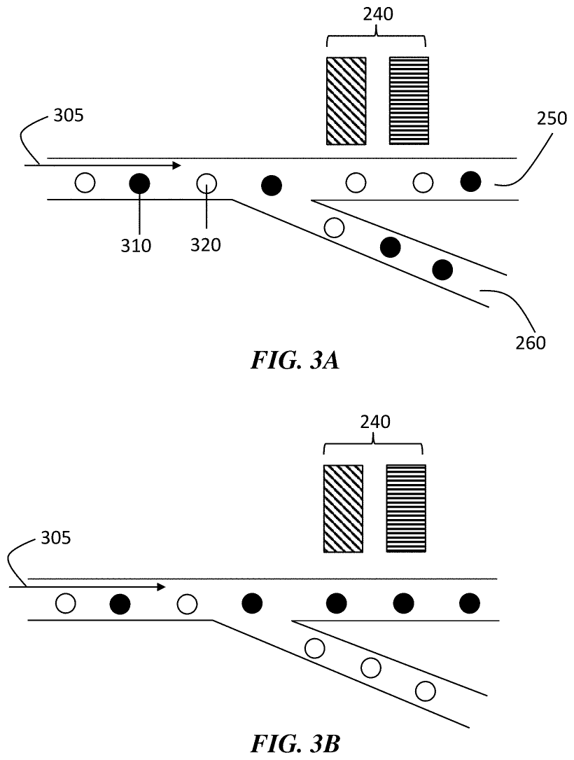

Droplets that do not contain target enzymes of interest (e.g., enzymes with one or more characteristics) may not generate the appropriate luminescent signal. For example, in FIG. 3A, droplets that do not contain a target enzyme of interest may not be attracted to sorting electrodes 240 and will pass to a waste channel 260. In some cases, however, droplets not attracted to the sorting electrodes 240 may pass to a different portion of the system or microfluidic device, such as an upstream portion for further incubation (not pictured). Additional details regarding droplet sorting are described elsewhere herein.

It should be noted that the various regions, areas, and/or chambers described herein may be joined and/or in fluid communication with one another via one or more channels, chambers or conduits between the regions, areas, and/or chambers through which droplets and fluids may flow. For example, a first channel (e.g., a collection channel) may connect to and be in fluid communication with a sorting region, and a second channel (e.g., a waste channel) may also connect to and be in fluid communication with the sorting region. However, additional channels or conduits may be present. For example, a channel may connect an incubation region with a sorting region, a sorting region may have a channel connecting an upstream position to allow droplets more time for incubation, and/or a conduit can connect a merging region to an incubation region. Of course, other connections between regions, areas, and/or chambers (and/or other channels within the system) as possible, and those ordinary skill in the art in view of the teachings of this disclosure will be capable of selecting appropriate channels or conduits between the various regions, areas, and/or chambers described herein.

In some embodiments, microfluidic device 200 further comprises a collection chamber 252 and a waste chamber 262. The collection chamber is configured to store droplets containing the target enzyme (e.g., with one or more characteristics) performing at or above a desired threshold, while the waste chamber is configured to store droplets not containing the target enzyme or not containing the target enzyme with one or more characteristics. The collection chamber may further be configured to (or comprise reagents for) sequencing each target enzyme. This may advantageously allow a user to sequence enzyme variants that perform better (e.g., with a faster reaction rate) than other variants (e.g., enzyme variants collected in the waste chamber that did not perform with a fast enough reaction rate).

As mentioned above, the systems and methods described herein may be used to screen, detect, sort, and/or isolate droplets (or cells contained within droplets) that contain certain target enzymes, for example, when those target enzymes perform at a particular rate or with a particular activity. For example, according to some embodiments, the systems and methods disclosed may be used to screen an enzyme library containing a plurality of enzyme variants. Each enzyme variant may catalyze a reaction (e.g., converting a reaction substrate into a target product) at a different rate, and it may be desired to screen and/or isolate enzyme variants that, for example, react at a faster rate. Thus, droplets containing the target enzyme that generate (or quench) a luminescent signal based, at least in part, on the activity of the enzyme variant may be screened and/or sorted, while droplets containing a target enzyme that does not have the desired enzymatic activity (and hence does not generate or quench a luminescent signal) may be avoided, rejected, or provided with more incubation time.

As mentioned above, the systems and methods of the disclosure allow screening of an enzyme for one or more characteristics. In one example, the one or more characteristics of the enzyme is enzyme activity, enzyme stability at various temperatures and/or pH, enzyme specificity, stereoselectivity, the ability to convert a non-natural substrate. To determine enzyme stability, the droplets comprising the enzyme may be incubated at various temperatures or pH and screening the enzymes for activity. Other examples of enzyme characteristics that can be screened include but are not limited to chemoselectivity, regioselectivity, stability in the presence of a solvent, temperature stability, solubility, expression level in a single host organism, expression level in multiple host organisms, discovery of a new enzyme and discovery of a new function of a known enzyme. Of course, other characteristics are possible.

The present disclosure may also provide a method for screening an enzyme for increasing conversion of its natural substrate, increasing conversion of its non-natural substrate, increasing catalytic activity, increasing stereoselectivity, increasing thermal stability, increasing stability in a predetermined pH range, or a combination thereof. The present disclosure may also provide a method for increasing chemoselectivity, increasing regioselectivity, increasing stability in the presence of a solvent, such as a water miscible solvent, increasing solubility, increasing expression level in a signal host microorganism, increasing expression level in multiple host organisms, discovery of a new enzyme, discovery of a new function of a known enzyme, protein and enzyme engineering, directed enzyme evolution or rational design enzyme engineering.

As mentioned above, in some examples, the enzyme (e.g., the target enzyme) is a redox cofactor-dependent enzyme. In some such examples, the enzyme is a redox cofactor-independent enzyme, and the signal is emitted when the redox cofactor is oxidized or reduced when the target product is further converted to a subsequent product by the detection enzyme. More than one detection enzyme can be used, such as in a cascade of reactions. It will be generally understood by a person skilled in the art that cascade reactions may involve multiple steps of enzymatic reactions. Detection enzymes may be cofactor-dependent enzymes or co-factor independent enzymes. When only one detection enzyme is used, the detection enzyme is generally a cofactor-dependent enzyme. When more than one detection enzyme is used, at least one of the detection enzymes is a cofactor-dependent enzyme. In other examples, the enzyme is a redox cofactor-independent enzyme.

Examples of detection enzymes include but are not limited to alcohol dehydrogenase (ADH), aldehyde dehydrogenase, amine dehydrogenase. It will generally be understood that the specific detection enzyme selected depends on the target product produced. If more than one detection enzymes are used, one or more detection enzymes that are dependent on the specific cofactors may be selected.

In some examples, the enzyme is expressed in a host cell and the plurality of droplets further comprise the host cell or fragments thereof. The host cell may be a eukaryotic, prokaryotic, archaeal, fungal, protozoan, mammalian, bacterial, yeast, plant, or algal cell. In one example, the host cell is an Escherichia coli cell. Fragments of the host cell may be generated by lysis of the host cell.

In some embodiments, a target product is converted to a subsequent product by a cofactor-dependent detection enzyme. The signal is emitted when the cofactor is oxidized or reduced when the target product is converted to the subsequent product by the cofactor-dependent detection enzyme.

In various embodiments, a target product may be converted to a first subsequent product by a first detection enzyme, where the first detection enzyme is a cofactor-independent enzyme, and the first subsequent product is further converted to a second subsequent product by a second detection enzyme wherein the second detection enzyme is a cofactor-dependent enzyme. The signal is emitted when the cofactor is oxidized or reduced when the first subsequent product is further converted to a second subsequent product by the second detection enzyme.

In some embodiments, the enzyme (e.g., target enzyme) is a redox cofactor-dependent enzyme, and the signal is emitted when the redox cofactor is oxidized or reduced when the reaction substrate is converted to the target product by the enzyme.

In some embodiments, the enzyme (e.g., target enzyme) is an intracellular enzyme, a surface displayed enzyme or an extracellular enzyme. It will be understood to those of skill in the art that any enzyme may be screened by the methods of the disclosure. The enzymes may be natural or synthetic enzymes. In some examples, the enzyme is Candida parapsilosis (CpSADH), epoxide hydrolase from Sphingomonas sp. HXN-200 (SpEH), styrene oxide isomerase (SOI), P450 monooxygenase (P450pyr), amine dehydrogenase from Rhodococcus (AmDH), or butanediol dehydrogenase (BDHA), without limitation.

In some embodiments, the enzyme (e.g., the target enzyme) is an enzyme variant selected from an enzyme variant library or from a library of various single enzymes. The library of enzyme variants may be a pre-existing library of enzyme variants or may be a library of enzyme variants generated based on an enzyme that has been screened with the methods of the disclosure. It will be understood that the screened enzyme from the present disclosure can be used to generate an enzyme variant library where the variants of this generated library are subjected to another round of screening using the methods of the disclosure. Multiple rounds of screening and library generation may be performed.

In another embodiment, the enzyme is an enzyme selected from a group of different enzymes. For example, the group of different enzymes may be a metagenomics library or synthetic sequences from databases. The methods of the disclosure can therefore be used to screen for enzyme variants for protein and enzyme engineering or directed enzyme evolution, or to screen for different enzymes for enzyme discovery.

In some embodiments, the systems and methods include one or more microfluidic droplets. The droplets used in the method of the disclosure may be water-in-oil droplets, water-in-oil-in-water droplets, oil-in-water droplets, and hydrogel droplets.

The droplets described herein may be of any suitable size (e.g., a diameter). In some embodiments, a diameter of a droplet is greater than or equal to 10 μm, greater than or equal to 20 μm, greater than or equal to 30 μm, greater than or equal to 40 μm, greater than or equal to 50 μm, greater than or equal to 60 μm, greater than or equal to 70 μm, greater than or equal to 80 μm, or greater than or equal to 90 μm, or greater than or equal to 100 μm. In some embodiments, a diameter of a droplet is less than or equal to 100 μm, less than or equal to 90 μm, less than or equal to 80 μm, less than or equal to 70 μm, less than or equal to 60 μm, less than or equal to 50 μm, less than or equal to 40 μm, less than or equal to 30 μm, less than or equal to 20 μm, or less than or equal to 10 μm. Combinations of the above-referenced ranges are also possible (e.g., greater than or equal to 10 μm and less than or equal to 100 μm). Other ranges are possible.

Various embodiments described herein may include one or more cofactors. In some embodiments, the cofactor may undergo one or more chemical reactions (e.g., oxidation-reduction reactions) to form a redox couple of the cofactor. In some embodiments, the cofactor may generate a luminescent signal directly or may indirectly participate in one or more chemical reactions to generate a luminescent signal or cause another chemical species to generate the luminescent signal. In some embodiments, the cofactor is selected from the group consisting of NAD⁺, NADH, NADP⁺, NADPH, NAD⁺ and NADP⁺, and NADH and NADPH. However, other cofactors are possible as this disclosure is not so limited. It will generally be understood that when the redox cofactor is NAD+ or NADP+ or both, the signal is emitted when NAD+ or NADP+ or both is reduced. Similarly, when the redox cofactor is NADH or NADPH or both, the signal is emitted when NADH or NADPH or both are oxidized. In one example, the signal detected is the fluorescence of NADPH or NADH or both.

As mentioned elsewhere herein, each droplet of the plurality of droplets may also further comprise one or more detection reagents that react with the oxidized or reduced redox cofactor to produce the emitted signal. A person skilled in the art would understand that the detection reagents used depends on the redox cofactor in the droplet. In some embodiments, the detection reagent is a fluorescent dye configured to react with the cofactor (or a redox couple of the cofactor) in order to generate the luminescent signal. In some embodiments, the detection reagent is a detection enzyme configured to react with a target product of the target enzyme. In some embodiments, the detection reagent comprises a quantum dot configured with a molecule (e.g., a ligand, L) that may interact with the cofactor (or a redox couple of the cofactor) in order to generate the luminescent signal.

Any suitable fluorescent dye capable of interacting with a cofactor (or a redox couple of the cofactor) may be used. In one example, when the redox cofactor is NADH or NADPH or both, the one or more detection reagents is resazurin and diaphorase. The NADH and/or NADPH reacts with resazurin in the presence of diaphorase to give a resorufin. A signal emitted by resorufin is then detected. However, other fluorescent dyes are possible, as this disclosure is not so limited.

A detection enzyme may be selected such that it reacts with a target product of the target enzyme reaction. In one example, when the redox cofactor is NADH or NADPH or both, the one or more detection reagent is proluciferin, luciferase, a reductase, ATP and Mg²⁺. Proluciferin is reduced to D-luciferin by a reductase in the presence of NADH and/or NADPH and the D-luciferin is converted in the presence of Mg²⁺ and ATP to oxyluciferin. A signal emitted by oxyluciferin is then detected. However, other detection reagents (e.g., detection enzymes) are possible as this disclosure is not so limited. Those of ordinary skill in the art in light of the teachings of the present disclosure will be capable of selecting appropriate detection reagents in order to generate a luminescent signal for screening one or more target enzymes.

In some embodiments, the detection reagent comprises a quantum dot (i.e., a nanocrystal). The quantum dot may be configured or functionalized to generate or quench a fluorescent signal upon reaction or interaction with a cofactor or a redox couple of the cofactor. For example, in some embodiments, the redox cofactor is NADH, NADPH, NAD+, NADP+, NAD+ and NADP+ or NADH and NADPH, the one or more detection reagents is a quantum dot. Non-limiting examples of quantum dots include CdS, CdSe, ZnS, ZnSe, and/or CdZnS. Other quantum dots are possible.

The quantum dot may be functionalized (e.g., surface functionalized) or conjugated with a ligand L, which may react (e.g., oxidation, reduction) or interact with the cofactor to generate or quench a fluorescent signal. Without wishing to be bound by any particular theory, the ligand may interact with the quantum dot via a fluorescence resonance energy transfer (FRET) mechanism. A cofactor may come into proximity or contact with the ligand and/or quantum dot and activate or deactivate FRET between the quantum dot and the ligand, thereby generating (or quenching) a signal, which may be used by a detector (e.g., a detector in a detection area or sorting region, a detector upstream the sorting region) to initiate or stop droplet sorting.

In an exemplary embodiment, the ligand of the quantum dot comprises Nile blue (i.e., [9-(diethylamino)benzo[a]phenoxazin-5-ylidene]azanium sulfate) or an analog thereof. In some embodiments, the ligand comprises formula (I):

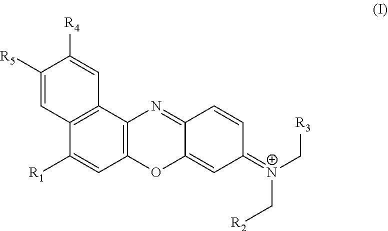

where R₁ is selected from the group including or consisting of amido, amino, and diamino; R₂ is selected from the group including or consisting of alkyl (e.g., methyl, ethyl), carboxyl (e.g., acetyl), and sulfonyl (e.g., —CH₂CH₂SO₃H); R₃ is selected from the group including or consisting of alkyl, carboxyl (e.g., acetyl), and sulfonyl (e.g., —CH₂CH₂SO₃H); R₄ is selected from the group including or consisting of hydrogen and hydroxyl; and Rs is selected from the group including or consisting of hydrogen and sulfonyl (e.g., —SO₃H). In some embodiments, R₁ may also bond or conjugate the ligand to the quantum dot. In some embodiments, an analog of Nile blue comprises formula (I).

In some embodiments, the detection reagent comprises a biosensor, such as a protein biosensor, as one non-limiting example of a biosensor. The biosensor may be configured to react with the cofactor (or a redox couple of the cofactor) to generate (or quench) a luminescent signal. The generation (or quenching) of the luminescent signal may be used to detect, screen, and/or isolate droplets containing the target enzyme. In one example, the protein biosensor comprises a fluorescent core domain and a bipartite NAD+ and/or NADP+ binding domain. The fluorescent core domain has an excitation at 488 nm and an emission at 520 nm. In the presence of NAD+ and/or NADP+, the fluorescence from the core domain is quenched by binding of the NAD+ and/or NADP+ to the bipartite binding domain.

In some embodiments, the activity of the enzyme (e.g., the target enzyme) within a droplet results directly or indirectly in the generation of a luminescent signal. The luminescent signal may be a fluorescence signal, a bioluminescence signal or a chemiluminescence signal. In some embodiments, the luminescent signal may be used to screen, detect, and/or or isolate droplets containing the enzyme of interest (e.g., the target enzyme). For example, droplets containing a target enzyme in which the target enzyme carries out the desired reaction may generate a luminescent signal and be sorted into a collection chamber. In some cases, however, it is possible for the absence of a luminescent signal relative to a reference signal to trigger screening and/or sorting.

In some embodiments, the reference signal is a signal from a reference enzyme. The reference enzyme may be a wild-type enzyme, a mutated enzyme, a chimeric enzyme or enzyme variant. In some embodiments, the reference signal is a signal of a known intensity or level, for example, a fluorescence or a bioluminescence signal from a fluorophore. In some embodiments, the reference signal is a signal having a predetermined intensity or level that is greater than or less than a reference. In some embodiments, a derived signal (e.g., a ratio of a first and second luminescent signal) is determined. In some embodiments, the derived signal is compared to a reference signal. In other embodiments, the reference signal may be no signal, an undetectable signal or a signal that is indistinguishable from noise.

In some embodiments, two or more distinct luminescent signals may be generated within the droplet (e.g., from a detection reagent within the droplet, from a cofactor within the droplet). That is, a first luminescent signal of a first wavelength and a second luminescent signal of a second wavelength may be generated within a droplet wherein the first wavelength and the second wavelength are different. Advantageously, generating two or more distinct luminescent signals may, for example, increase the sensitivity for detecting target enzymes by reducing the background noise generated from excitation sources (e.g., an excitation light, an excitation laser) in the case where the luminescence is fluorescence, and an excitation source may be used to generate a fluorescent signal.

In some embodiments, two (or more) distinct luminescent signals (e.g., generated within the same or different droplet) may determine two (or more) distinct characteristics of a droplet. For example, a first luminescent signal may determine the number of cells within the droplet, and a second luminescent signal may indicate the presence (e.g., the concentration) of the cofactor within the droplet. Other characteristics may also be determined. The two or more signals may be detected using a single detector or two or more detectors as described herein. In some embodiments, the first and/or second luminescent signals (or derivatives thereof) may indicate a characteristic of the droplet and/or signal a function of the system or method involving the droplet. In some embodiments, a ratio of the first and second luminescent signal may indicate a characteristic of the droplet and/or or signal a function of the system or method involving the droplet, such as how the droplet is sorted. Other functions may be signaled.

As described elsewhere herein, a detector may be used to detect a luminescent signal (e.g., a fluorescent signal). A detector may measure the intensity or a magnitude of the intensity of the luminescent signal. While the detector may be located within a detection area of the sorting region, one or more detectors may also be located in an upstream or downstream position of the sorting region. The one or more detectors (or a controller operatively associated with the detector(s)) may digitize or otherwise electronically transform the signals, e.g., a first luminescent signal, a second luminescent signal, and/or a ratio of the first luminescent signal and the second luminescent signal, which may provide information, for example, for sorting of the droplets as discussed in more detail elsewhere herein. For instance, the first luminescent signal, second luminescent signal, or a derivative (e.g., a ratio) of the first and second luminescent signals (e.g., from a single droplet) may be transmitted to a feedback control or controller (e.g., a microcontroller) which compares the luminescent signal(s) to a pre-programmed or predetermined signal. In some such embodiments, the comparison may activate or deactivate droplet sorting or some other function or step of the fluidic system or disclosed method. For example, the comparison may determine whether a voltage is to be applied to a set of sorting electrodes to begin sorting (e.g., via electrophoretic force) a droplet of interest (e.g., containing a target enzyme). In some embodiments, the voltage applied to a set of sorting electrodes determines if a droplet of interest is sorted into a first channel (e.g., a collection channel) or a second channel (e.g., a waste channel). In some embodiments, a voltage is applied to a set of merging electrodes to facilitate merging of one or more reagents (e.g., detection reagents) into a droplet. Other functions may be enabled by a comparison of the signals, e.g., the first luminescent signal, the second luminescent signal, and/or a ratio of the first and second luminescent signal, as this disclosure is not so limited.

For example, in some embodiments, the first and/or second luminescent signals (or derivatives thereof) may be transmitted to a feedback control or controller (e.g., a microcontroller) to determine whether the flow rate of the droplets (e.g., in the incubation region) should be modulated. For instance, the incubation time may be modulated by such a control mechanism where the feedback control or controller is electronically connected to a flow source (e.g., a pressure source such as a pump or vacuum). The signals detected in the droplets can thus provide feedback control of the flow rate and/or incubation time of the droplets.

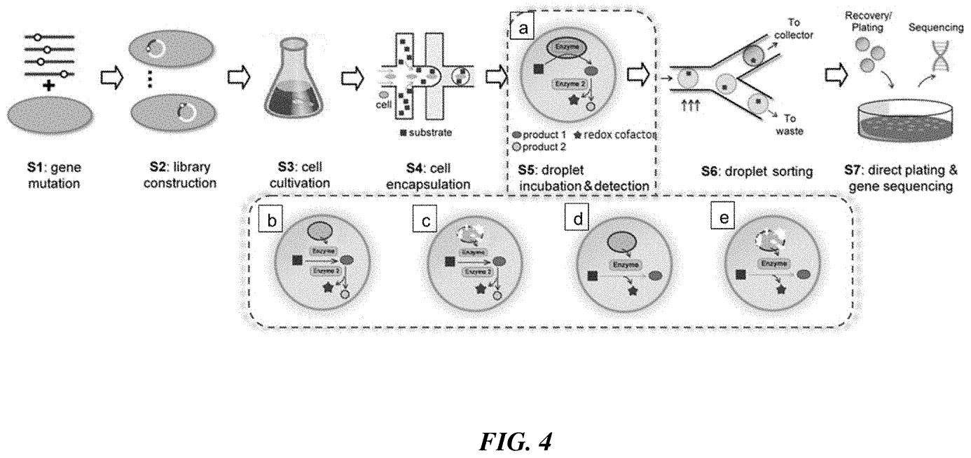

As mentioned above, the systems and methods described herein may be used to screen enzymes (e.g., enzyme variants each within a plurality of cells, enzyme variants each within a plurality of droplets) within droplets that generate (or quench) a luminescent signal while rejecting droplets that do not contain the target enzyme, or where the target enzyme does not have one or more characteristics and hence does not generate (or quench) a luminescent signal. In order to detect droplets containing the target enzyme(s), a detector may sense or detect a luminescent signal or a change in the luminescent signal at some particular threshold (e.g., a predetermined value). Droplets above this threshold may be collected, while droplets below this threshold may go to waste (or vice versa) or back to an upstream channel, such an upstream incubation region or incubation channel. In some embodiments, the detector is coupled to a set of sorting electrodes, which may be “OFF” below the particular threshold and “ON” above the particular threshold. In this way, droplets above the particular threshold may be sorted from droplets below the particular threshold. FIG. 4A and 4B illustrate this feature. FIG. 4A is a schematic of a portion of the sorting region. In FIG. 4A, the sorting electrodes 340 are OFF, such that a droplet of interest 410 and an undesired droplet 420 are not sorted as they flow towards collection channel 350 and waste channel 360. However, in FIG. 4B, when the electrodes are turned on (e.g., by a signal received from the detector), the droplets of interest 410 are flowed to collection channel 360 via an attractive force generated by sorting electrodes 340, while the undesired droplets 420 are not attracted towards the sorting electrodes 340 and flow, instead, to waste channel 360. Without wishing to be bound by any particular theory, the electrodes may attract (e.g., via electrostatic forces, dielectrophoretically) droplets above the particular threshold to a collection channel or collection chamber, while droplets below the particular threshold are not sorted to the collection channel or collection chamber.

Any suitable detector may be used to detect the luminescent signal. The detector may be configured to detect electromagnetic radiation (i.e., light) such as the electromagnetic radiation generated by luminesce (e.g., fluorescence, bioluminescence, chemiluminescence). In some embodiments, the detector is configured to detect more than one wavelength. In an exemplary embodiment, the detector comprises a photomultiplier (PMT) detector. In some embodiments, the detector is capable of and/or configured to receive two or more luminescent signals, such as a dual-channel fluorescent detector. However, other detectors are possible. In some embodiments, the detector may also comprise an excitation source, for example, to subsequently generate a fluorescent signal after exciting a molecule capable of fluorescence. The detection may further comprise or be configured to connect to a lens or a camera (e.g., a monochrome camera), which may collect or help collect an intensity of the luminescent signal.

In some embodiments, the detector may be configured to receive two or more separate signals from the same (or different) droplet. For example, the detector may be configured to receive at least two signals (e.g., at two different wavelengths) from a droplet, which may be used to provide more information for sorting droplets. Details of receiving two or more separate signals are discussed in more detail below and elsewhere herein.

In some embodiments, the detector may be configured to transform (e.g., digitize) a signal to electronically communicate with other components of the system. In some such embodiments the detector may comprise or be in electronic communication with one or more controllers, which may further be in electronic communication with other components of the system. In some embodiments, the detector (or a controller of the detector or in electronic communication with the detector) is in electronic communication with a set of sorting electrodes and is further configured to turn on or turn off the electrodes (i.e., by applying a voltage) to start or stop sorting of droplets above or below a particular detection threshold. Those skilled in the art based on the teachings of the present disclosure will be capable of selecting a particular detection threshold for desired screening, sorting, and/or isolation of target enzymes.

In some embodiments, the systems and methods may involve a feedback control in which a signal from one position in the system, such as a downstream position (e.g., a detection area), may be used to provide information or to control (at least in part) a function in another position in the system, such as an upstream position (e.g., incubation, flow rate). In some instances, the systems and methods may involve a feedback control in which a signal from an upstream position (e.g., a second detector upstream of the first detector) may be used to provide information or control (at least in part) to a function in a downstream position. For example, a detector (e.g., a first detector) may obtain a signal (e.g., a luminescent signal, a fluorescent signal) from a downstream position and function of an upstream position (e.g., flow rate, incubation time) may be modified based (at least in part) from the downstream signal. As another example, a second detector at an upstream position may receive a signal (e.g., a reference signal) and the detection area (e.g., a first detector within the detection area) may receive the signal from the second detector and, modify or control a downstream function (e.g., detecting, sorting) based (at least in part) on the signal received from the second detector. Of course, other feedback configurations are possible. In some embodiments, a controller (e.g., a microcontroller) may be in electronic communication with one or more detectors and/or one or more components to be controlled in the system, and may convey a signal and/or control one or more functions of embodiments described herein.

In some embodiments, the feedback control involves the detection of one or more signals (e.g., luminescent signals), events, or processes occurring in the system or method. Detection may involve, for example, determination of at least one characteristic of a target enzyme, a component within a fluid, a characteristic of the fluid, interaction between components within regions of the system, and/or a condition within a region (e.g., a sorting region) of the system (e.g., flow rate, sorting threshold, temperature, pressure). For instance, detection may involve detecting a luminescent signal of a droplet, a concentration of one or more components in a droplet, a volume of one or more fluids inside or outside a droplet, a flow rate of one or more fluids (or a droplet within a fluid), and an average time period between the detection of a first luminescent signal and a second luminescent signal, without limitation. Detection of the one more characteristics, conditions, or events may, in some embodiments, result in the generation of one or more signals, which can be optionally further processed and transmitted to the control system (e.g., using a microcontroller). As described in more detail herein, the one or more signals may be compared with one or more other signals, values or thresholds pre-programmed into the feedback control, and may be used to provide feedback to the systems or methods.

In some embodiments, detection of signals from one or more detectors is performed continuously. In other embodiments, detection of signals from one or more detectors is performed periodically. In some embodiments, detectors are positioned at multiple or various regions in the system (e.g., adjacent to multiple incubation chambers) to monitor the droplets as a function of time. For instance, cell growth as a function of time may be determined. In some cases, the signals detected may be used to provide feedback control of another process within the system (e.g., to control incubation time, flow rate, and/or sorting).

A variety of signals or patterns of signals can be generated and/or determined (e.g., measured) using the systems and methods described herein. In some embodiments, the signal is a luminescent signal or a fluorescent signal. In one set of embodiments, a signal includes an intensity component. Intensity may indicate or be used to indicate, for example, one or more of: the concentration of a component in a droplet, an indication of the type of enzyme being detected, the amount of a component in a droplet, and the volume of a droplet. In some embodiments, the intensity may include a signal (e.g., a luminescent signal) from one target enzyme or detection reagents within a droplet. In other embodiments, the intensity may include a signal from two or more target enzymes or detection reagents within a droplet. In some such embodiment the intensity of the signals may be an average of each individual luminescent signal within the droplet. In some cases, intensity is a luminescent intensity from a luminescent signal of a droplet. Other intensity determinations are possible.

In some embodiments, a frequency of signals may be generated and/or determined from one or more luminescent signals. For example, a series of signals each having an intensity (e.g., above or below a threshold intensity) may be measured by a detector. This number may be compared with a number of signals or values (having the intensity above or below the threshold intensity) pre-programmed into the feedback control or other unit (e.g., a microcontroller). Based at least in part on this comparison, the feedback control may initiate, halt, or change a function such as the modulation of fluid flow or activation/deactivation of droplet sorting within the system or method.

In some embodiments, a duration of a signal (e.g., a luminescent signal) is generated and/or determined. The duration of a signal may indicate or be used to indicate, for example, one or more of: sorting, the flow rate of a fluid, a characteristic of a component within a fluid (e.g., how long a component has a certain characteristic or activity, such as chemiluminescence, fluorescence, and the like), and how long a particular droplet has been positioned in a specific region (e.g., an incubation region) of the system or method.

In some embodiments, a position of a signal in time relative to a second position in time or relative to another process or event (e.g., that has occurred in the system or method) is generated and/or determined. For example, a detector may detect when a certain droplet passes across the detector (e.g., a droplet containing an enzyme with one or more characteristics), and the timing of this signal may be related to a second position in time (e.g., when detection was initiated; a certain amount of time after a process has occurred, etc.). In another example, a detector may detect when a certain droplet passes across the detector after (or before) a component of the system (e.g., a set sorting electrode) has been activated

In another set of embodiments, an average time between signals or events is generated and/or determined. For instance, the average time period between two signals may be measured, where each of the signals may independently correspond to one or more characteristics or conditions described herein. In other embodiments, the average time between the first and the last of a series of similar signals is determined (e.g., the average time between a series of wash fluids passing across a detector).

In some embodiments, a pattern of signals is generated and/or determined. The pattern of signals may include, for example, at least two of (or, in other embodiments, at least three of, or at least four of) an intensity of a signal, a frequency of signals, a duration of a signal, a position of a signal in time relative to a second position in time or relative to another process or event occurring (or has occurred) in the microfluidic system, and an average time period between two or more signals or events. In other embodiments, the pattern of signals comprises at least two of (or, in other embodiments, at least three of, or at least four of) an intensity of a first signal, a duration of the first signal, a position of the first signal in time relative to a second position in time; an intensity of a second signal, a duration of the second signal, a position of the second signal in time relative to a second position in time, and an average time period between the first and second signals. The pattern of signals may indicate, in some embodiments, whether a particular event or process is taking place properly within the microfluidic system. In other embodiments, the pattern of signals indicates whether a particular process or event has occurred in the microfluidic system. In yet other embodiments, a pattern of signals can indicate a particular sequence of events.

A variety of signals or patterns of signals, such as those described above and herein, can be generated and/or determined and can be used alone or in combination to provide feedback for controlling one more or more process, such as modulation of sorting within the system. That is, the feedback control or any other suitable unit may determine, in some embodiments, whether to activate or deactivate droplet sorting within the system based at least in part on the pattern of signals. For example, determination of whether to sort may be based at least in part on a pattern of signals that includes an intensity of a first signal (e.g., a first luminescent signal) and a position in time of the first signal relative to a second position in time may involve the use of both of these pieces of information to make a decision on whether or not to sort droplets into collection or waste. For instance, these signals may be compared to one or more reference signals (e.g., a threshold intensity or intensity range, and a threshold position in time or range of positions in time, relative to a second position in time) that may be pre-programmed or pre-set into the feedback control. If each of the measured signals falls within the respective threshold values or ranges, a decision on whether to sort the droplets may be made. Only one of the parameters to be considered (e.g., only an intensity of the first signal or only a position in time of the first signal) that meets a threshold value or range may not be sufficient information to make a decision on whether or not to sort the droplets, because it may not give enough information about the luminescent signal or component(s) that gave rise to the signal(s) for the purposes described herein. For example, in some cases the luminescent signal detected may not be sufficiently identified for the purposes described herein unless a pattern of signals is taken into consideration.

In some embodiments, one or more measured signals is processed or manipulated (e.g., before or after transmission, and/or before being compared to a reference signal or value). It should be appreciated, therefore, that when a signal is transmitted (e.g., to a control system), compared (e.g., with a reference signal or value), or otherwise used in a feedback process, that the raw signal may be used or a processed/manipulated signal based (at least in part) on the raw signal may be used. For example, in some cases, one or more derivative signals of a measured signal can be calculated (e.g., using a differentiator, or any other suitable method) and used to provide feedback. In other cases, signals are normalized (e.g., subtracting a measured signal from a background signal). In one set of embodiments, a signal comprises a slope or average slope, e.g., an average slope of intensity as a function of time.

A feedback control may be used to continually sort droplets of interest into a first channel (e.g., a collection channel) while sorting undesired droplets into a second channel (e.g., a waste channel, a channel to an upstream position of the fluidic system). For example, a detector located an upstream position of the sorting region may detect a first luminescent signal of one or more droplets indicative of the presence or number of cells within the droplet. The detector may also detect a second luminescent signal indicative of characteristic of a cofactor (e.g., a cofactor concentration). Optionally, the detector (or a controller operatively associated with the detector) may calculate a derived signal involving average signal intensity per droplet (i.e., total signal intensity of the droplet divided by the number of cells in the droplet). In some embodiments, a ratio of the first and second luminescent signal (or an intensity of the signal) is determined. The signal (or derived signal) from the upstream detector may then be transmitted to downstream position (e.g., via a microcontroller). In some embodiments, the first luminescent signal, the second luminescent signal and/or the derived signal are compared to pre-programmed or pre-determined signal (e.g., pre-programmed into the microcontroller). The microcontroller may then determine a voltage or actuate a set of electrodes to begin sorted or to stop sorting based, at least in part, on the signal from the upstream detector.

In some embodiments, the detector is part of an analyzer separate from the system (or “off-chip), and the system may be configured to be inserted into or otherwise be analyzed by the analyzer. The analyzer may be used in a variety of ways to process and analyze a system placed within the analyzer. In some embodiments, once a mechanical component configured to interface with the system is properly loaded in the analyzer, the analyzer reads and identifies information about the system (e.g., a luminescent signal). The analyzer may be configured to compare the information to stored data (e.g., data stored within a microcontroller, data stored within a feedback control system). In some embodiments, the analyzer comprises a detector. By way of illustration and not limitation, the analyzer may receive information from a luminescent signal and determine (alone or in part with other parts of the system, such as the microcontroller or a feedback control) if sorting (within the sorting region) should be activated or if longer incubation time is required by the system.

The sorting electrodes may apply a voltage to attract or urge droplets of interest (e.g., droplets containing a target enzyme of a particular characteristic) towards a first channel (e.g., a collection channel). In some embodiments, a voltage of greater than or equal to 1 V, greater than or equal to 10 V, greater than or equal to 25 V, greater than or equal to 50 V, greater than or equal to 100 V, greater than or equal to 250 V, greater than or equal to 500 V, greater than or equal to 750 V, or greater than or equal to 1 kV is applied by the sorting electrodes. In some embodiments, a voltage of less than or equal to 1 kV, less than or equal to 750 V, less than or equal to 500 V, less than or equal to 250 V, less than or equal to 100 V, less than or equal to 50 V, less than or equal to 25 V, less than or equal to 10 V, or less than or equal to 1 V. Combinations of the above-referenced ranges also possible (e.g., greater than or equal to 1 V and less than or equal to 1 kV). Other ranges are possible.

Droplet sorting may be conducted at any suitable speed for differentiating droplets of interest (e.g., droplets containing a target enzyme with one or more characteristics) from undesired droplets. In some embodiments, the droplet sorting speed is greater than or equal to 10 droplets/second, greater than or equal to 20 droplets/second, greater than or equal to 50 droplets/second, greater than or equal to 100 droplets/second, 250 droplets/second, greater than or equal to 250 droplets/second, greater than or equal to 500 droplets/second, greater than or equal to 750 droplets/second, greater than or equal to 1,000 droplets/second, greater than or equal to 2,500 droplets/second, greater than or equal to 5,000 droplets/second, greater than or equal to 10,000 droplets/second, greater than or equal to 50,000 droplets/second, or greater than or equal to 100,000 droplets/second. In some embodiments, the droplet sorting speed is less than or equal to 100,000 droplets/second, 50,000 droplets/second, 10,000 droplets/second, 5,00 droplets/second, 1,000 droplets/second, less than or equal to 750 droplets/second, less than or equal to 500 droplets/second, less than or equal to 250 droplets/second, less than or equal to 100 droplets/second, less than or equal to 50 droplets/second, less than or equal to 20 droplets/second, or less than or equal to 10 droplets/second. Combinations of the above-referenced ranges are also possible (e.g., greater than or equal to 10 droplets/second and less than or equal to 100,000 droplets/second). Other ranges are possible.

Droplets may be isolated by various methods known in the art. In some examples, the droplets are isolated using fluorescence-activated droplet sorting (FADS), fluorescence-activated cell sorting (FACS) or magnetic-activated cell sorting (MACS). In other examples, the droplets are isolated using acoustic control techniques, magnetic control techniques, pneumatic control techniques, thermal control techniques, electric control techniques and combinations thereof.

In some embodiments, detection of a cofactor (e.g., NAD+, NADH) may occur at a concentration of greater than or equal to 1 μM, greater than or equal to 10 μM, greater than or equal to 100 μM, greater than or equal to 1000 μM, greater than or equal to 10 mM, or greater than or equal to 100 mM. In some embodiments, detection of a cofactor my occur at a concentration of less than or equal to 100 mM, less than or equal to 10 mM, less than or equal to 1000 μM, less than or equal to 100 μM, less than or equal to 10 μM, or less than or equal to 1 μM. Combinations of the above-referenced ranges are also possible (e.g., greater than or equal to 1 μM and less than or equal to 100 mM). Other ranges are possible.

As an example, NAD+, NADP+, NADH or NADPH can be detected at concentrations including but not limited to 1-10 μM, 1-100 μM, 1-1000 μM, 1 μM -10 mM, 100-1000 μM. It will be generally understood by a person skilled in the art that cofactors other than NAD⁺, NADH, NADP⁺, NADPH, NAD⁺ and NADP⁺, and NADH and NADPH can be used in the present disclosure. The detection method will vary according to the cofactor used and may be based on the spectral properties of the cofactors or the detection agents.

The cells may incubate directly within the fluidic system (e.g., microfluidic device), or separately from the fluidic system. In some embodiments, the cells may be cultured to facilitate cell growth and/or proliferation (e.g., within a droplet). In one example, the step of generation of or providing the plurality of droplets in the present methods of the disclosure further comprises incubating the host cell in each droplet with culture media in the droplet to increase the number of host cells, e.g., prior to adding the reaction substrate, detection enzyme, one or more detection reagents, lysis buffer, redox cofactor or combinations thereof. Selection of the culture conditions, including culture media, temperature, oxygen concentration, duration of culture will be understood to be dependent on the host cell and suitable for growth of the host cell. In some embodiments, this cell growth and/or proliferation occurs during continuous flow of the droplets or fluids in the incubation region. For instance, the droplets containing the cells may continuously flow from one incubation chamber to the next without needing to stop flow of the system.

In some embodiments, the droplets containing the cells can be incubated for greater than or equal to 1 minute, greater than or equal to 3 minutes, greater than or equal to 5 minutes, greater than or equal to 10 minutes, greater than or equal to 15 minutes, greater than or equal to 20 minutes, greater than or equal to 30 minutes, greater than or equal to 45 minutes, greater than or equal to 60 minutes, greater than or equal to 90 minutes, or greater than or equal to 120 minutes. In some embodiments, the droplets containing the cells can be incubated for less than or equal to 120 minutes, less than or equal to 90 minutes, less or equal to 60 minutes, less than or equal to 45 minutes, less than or equal to 30 minutes, less than or equal to 20 minutes, less than or equal to 15 minutes, less than or equal to 10 minutes, less than or equal to 5 minutes, less than or equal to 3 minutes, or less than or equal to 1 minute. Combinations of the above-referenced ranges are also possible (e.g., greater than or equal to 1 minute and less than or equal to 120 minutes). Other ranges are possible. In some embodiments, the configuration and/or geometry of the incubation area (e.g., the incubation chambers and/or channels within the incubation area) may determine the incubation time of the cells.

In some embodiments, the cells may grow or proliferate during incubation within the incubation region. In some embodiments, one or more cells may proliferate (e.g., reproduce, multiply) within a droplet. For example, one cell may be initially encapsulated in a droplet, and that one cell may grow into two or more cells. Of course, each daughter cell may also proliferate into two or more cells. In some embodiments, one cell may proliferate into at least 2 cells, at least 3 cells, at least 5 cells, at least 10 cells, at least 20 cells, at least 50 cells, at least 100 cells, at least 200 cells, at least 300 cells, or at least 500 cells. In some embodiments, no greater than 500 cells, no greater than 300 cells, no greater than 200 cells, no greater than 100 cells, no greater than 50 cells, no greater than 20 cells, no greater than 10 cells, no greater than 3 cells, or no greater than two cells are proliferated within a droplet. Combinations of the above-referenced ranges are also possible (e.g., at least 50 cells are proliferated within a droplet and no greater than 500 cells are proliferated within a droplet). Other ranges are possible. In some embodiments, the reagents within a droplet may be used to determine the proliferation of cells within the droplet.

The plurality of incubation chambers within the incubation region may have any suitable dimensions. The maximum transverse cross-sectional dimension of each chamber within the plurality of chambers may be the same or different. In some embodiments, a maximum transverse cross-sectional dimension of a chamber within the plurality of chambers is greater than or equal to 10 μm, greater than or equal to 20 μm, greater than or equal to 25 μm, greater than or equal to 30 μm, greater than or equal to 50 μm, greater than or equal to 100 μm, greater than or equal to 200 μm, greater than or equal to 300 μm, greater than or equal to 400 μm, greater than or equal to 500 μm, greater than or equal to 750 μm, greater than or equal to 800 μm, greater than or equal to 900 μm, or greater than or equal to 1000 μm. In some embodiments, a maximum transverse cross-sectional dimension of a chamber within the plurality of chambers is less than or equal to 1000 μm, less than or equal to 900 μm, less than or equal to 800 μm, less than or equal to 750 μm, less than or equal to 500 μm, less than or equal to 400 μm, less than or equal to 200 μm, less than or equal to 100 μm, less than or equal to 50 μm, less than or equal to 30 μm, less than or equal to 25 μm, less than or equal to 20 μm, or less than or equal to 10 μm. Combinations of the foregoing ranges are also possible (e.g., greater than or equal to 10 μm and less than or equal to 1000 μm). Other ranges are possible. A channel connecting two or more incubation chambers may also have dimensions of or within the foregoing ranges.

In some embodiments, a channel connecting two adjacent incubation chambers has a particular ratio relative to the incubation chamber. These ratios may be tuned in order to control the incubation time of the cells as they move through the incubation region and may also be used to control the relative sequence of droplets exiting the detection region relative to their order upon entering the detection region. In some embodiments, a ratio of a maximum cross-sectional dimension of an incubation chamber (e.g., a first incubation chamber and/or a second incubation chamber) to a maximum cross-sectional dimension of the channel is greater than or equal to 2:1, 3:1, 5:1, 10:1, 13:1, or 15:1. In some embodiments, a ratio of a maximum cross-sectional dimension of an incubation chamber to a maximum cross-sectional dimension of the channel is less than or equal to 15:1, 13:1, 10:1, 5:1, 3:1, or 2:1. Combinations of the foregoing ranges are also contemplated (e.g., greater than or equal to 2:1 and less than or equal to 15:1). Other ranges are possible as this disclosure is not so limited.

The incubation region may include any suitable number of incubation chambers. In some embodiments, the incubation region includes greater than or equal to 2 incubations chambers, greater than or equal to 3 incubation chambers, greater than or equal to 5 incubation chambers, greater than or equal to 10 incubation chambers, greater than or equal to 15 incubation chambers, greater than or equal to 20 incubation chambers, greater than or equal to 25 incubation chambers, greater than or equal to 30 incubation chambers, greater than or equal to 40 incubation chambers, or greater than or equal to 50 incubation chambers. In some embodiments, the incubation chamber includes less than or equal to 50 incubation chambers, less than or equal to 40 incubation chambers, less than or equal to 30 incubation chambers, less than or equal to 20 incubation chambers, less than or equal to 15 incubation chambers, less than or equal to 10 incubation chambers, less than or equal to 5 incubation chambers, less than or equal to 3 incubation chambers, or less than or equal to 2 incubation chambers. Combinations of the above-referenced ranges are also possible (e.g., greater than or equal to 2 incubation chambers and less than or equal to 50 incubation chambers). Other ranges are possible. The incubation chambers may be connected in series or in parallel.

In some embodiments, a bubble trap may be included within a system or microfluidic device positioned downstream the incubation region. The bubble trap may be used to remove gas bubbles formed, for example, during incubation of the cells in the plurality of incubation channels. Hence the bubble trap may be positioned and/or attached to a chamber or a channel downstream the incubation area, but, of course, may be positioned within any position of the fluidic system to facilitate removal of gas bubbles from a channel and/or a chamber. In some embodiments, the bubble trap comprises a membrane (e.g., a polydimethylsiloxane (PDMS) membrane) that may permit diffusion of gas (e.g., air) while preventing the flow of liquid through the bubble trap. Optionally, the bubble trap may further comprise a vacuum port (e.g., an inlet and/or an outlet) to facilitate the removal of gas by applying reduced pressure to the bubble trap

In some embodiments, the at least one or more of the plurality of droplets further comprises a lysis buffer. The lysis buffer lyses the host cell to release an intracellular enzyme which converts the reaction substrate to a target product. Examples of lysis buffers include but are not limited to proteinase-K, sodium dodecyl sulfate (SDS), Triton X-100, benzalkonium chloride, chlorhexidine digluconate, Lysozyme-Polymyxin B and phenol.

The isolated droplets may then be subjected to further assays or analyses. For example, the DNA or cells in the isolated droplets may be further recovered for further analyses. In one example, the recovered cells are cultured to allow growth of the host cell and the characteristics of the enzyme on a large scale, for example, in micro-litre to litre scale, can be tested. In another example, the DNA or the enzyme in the isolated droplet is sequenced to obtain the amino acid sequence, the polynucleotide sequence or both.

In one example, the sequenced enzyme is used to generate a new enzyme variant library. Methods to generate an enzyme library would be understood to one of skill in the art. For example, an enzyme library may be generated by random mutagenesis or targeted mutagenesis. Each variant in an enzyme library comprises a mutation in one or more amino acids compared to a reference protein or enzyme. The mutation may also be a silent mutation for codon optimization. The mutation may also be made to improve expression of a signal peptide that is subsequently removed.

As used herein, the term “enzyme” refers to a protein or protein-based molecule that is capable of catalyzing a chemical or biochemical reaction. Enzymes are totally or partially composed by one or more polypeptides but may also include nucleic acids. The catalytic function of an enzyme constitutes its “activity” or “enzymatic activity”. The specificity of an enzyme mainly depends on the characteristics of the active site, a region where it binds to the substrate before the substrate transformation into a product. An enzyme may be classified according to the type of catalytic function it carries out. For example, enzymes include, but are not limited to, oxidoreductases, transferases, isomerases, hydrolases, lyases and ligases. Enzymes may also include oxygenases, dehydrogenases and lipases. An enzyme may also be classified according to the type of its expression system in a host. For instance, enzymes may include intracellular enzymes, surface displayed enzymes and extracellular enzymes. Enzymes may be cofactor-dependent or cofactor-independent. A cofactor-dependent enzyme requires the presence of a cofactor in order for the enzyme to catalyze the reaction while a cofactor-independent enzyme does not require the presence of a cofactor.

As used herein, the term “substrate” refers to a substance that is capable of binding to or interacting with the active site of an enzyme. The enzyme performs its catalytic activity on the substrate to generate a product.

As used herein, the term “cofactor” refers to any molecule, other than the substrate(s), that is required for an enzyme to carry out its enzymatic activity. Cofactors include, but are not limited to, inorganic ions, coenzymes, or other factors necessary for the activity of the enzymes. Redox cofactors are cofactors required by oxidoreductases to catalyse the enzymatic reaction. Examples of redox cofactors include NADP+ (nicotinamide adenine dinucleotide phosphate), NADPH (i.e. reduced form of NADP+), NAD+ (nicotinamide adenine dinucleotide), and NADH (i.e. a reduced form of NAD+). Other redox cofactors include but are not limited to flavin adenine dinucleotide (FAD and FADH2), flavin mononucleotide (FMN and FMNH2), Vitamin B12, and coenzyme Q as well as synthetic biomimetic cofactors. Insofar as it refers to the redox cofactors NAD+ and NADP+, it will generally be understood that “NAD(P)+” refers to “NAD+” and/or “NADP+”. Similarly, insofar as it refers to the redox cofactors NADH and NADPH, “NAD(P)H” would be understood to refer to “NADH” and/or “NADPH”.

As used herein, the term “screening” in the context of enzymes or enzyme variants refers to the analysis or testing of enzymes for one or more characteristics. Screening involves testing each sample of a group, such as different enzymes or enzyme variants, for a specific characteristic.

As used herein, the term “mutation” refers to, in the context of a polynucleotide, a modification to the polynucleotide sequence resulting in a change in the polynucleotide sequence compared to a reference polynucleotide sequence. Mutations to a polynucleotide sequence may or may not result in a change to the encoded amino acid sequence. For instance, a mutation that does not alter the amino acid sequence may be used for codon optimization for expression purposes. Mutations can be introduced into a polynucleotide through methods including, but not limited to, random mutagenesis, site-specific mutagenesis, oligonucleotide-directed mutagenesis, gene shuffling, directed evolution techniques, combinatorial mutagenesis and site saturation mutagenesis. The term “mutation” refers to, in the context of a protein, a modification to the amino acid sequence resulting in a change in the amino acid sequence of the protein compared to a reference amino acid sequence. The mutation may involve one or more amino acid residues and may be selected from the group consisting of substitution, insertion, deletion, truncation, and combinations thereof. The amino acid residue can be natural or non-natural. A “variant” or “mutant” as used herein is a protein or enzyme comprising a mutation of one or more amino acids as compared to a reference protein. A “variant” may also comprise additional amino acid extensions compared to a reference protein. The extensions may include signal peptides, protein tags and fluorescent tags. Examples of protein tags include His tag, FLAG tag, and Myc tag.

In the context of this application, the term “microfluidics” refers to the science of designing, manufacturing and formulating devices and processes that deal with small volumes of fluid which may be on the order of microliters, nanoliters or picoliters. Microfluidic devices or systems comprise channels on a micrometer scale and are capable of high levels of automation, reduced processing times and lower consumption of samples and reagents.

The term “fluorescence” as used herein refers to a type of luminescence in which an atom or molecule emits measurable radiation during quantum transition from a higher to a lower electronic state. A fluorophore is a fluorescent chemical compound that can re-emit light upon light excitation. Specifically, the fluorophore absorbs light energy of a specific wavelength, resulting in excitation of the fluorophore's electrons, and the fluorophore subsequently re-emits the absorbed light energy at a longer wavelength upon the electrons returning to their basic state.

As used herein, the term “chemiluminescence” refers to the production and emission of light by as a result of a chemical reaction. An example of chemiluminescence is bioluminescence.

As used herein, the term “bioluminescence” refers to the production and emission of light by a chemical reaction catalyzed by an enzyme, protein, protein complex or other biomolecule. In bioluminescence, the light-emitting compound is one that is found in living organisms. Examples of bioluminescent compounds include bacterial luciferase and firefly luciferase.

As used herein, the term “fluorescence resonance energy transfer” (FRET) refers to a process by which energy is transferred between two light-sensitive molecules such as chromophores and fluorophores when these molecules are in close proximity. A donor fluorophore, initially in its electronic excited state, may transfer energy to an acceptor fluorophore through non-radiative dipole-dipole coupling. The acceptor fluorophore then emits light at a different wavelength. This fluorescence emission wavelength may differ depending on the distance and status of the two fluorophores.

In context of this application, the term “quantum dot” refers to a semiconductor nanocrystal. Quantum dots may be classified based on their composition and structure and include but are not limited to core-type quantum dots, core-shell quantum dots and alloyed quantum dots. Quantum dots can also be water soluble or insoluble and can include a core of at least one of a Group II-VI semiconductor material, III-V semiconductor material, a group IV semiconductor material, or a combination thereof. Examples of quantum dots (QDs) include nanocrystals of CdSe, ZnS, ZnSe, ZnTe, CdS, CdTe, GaN, GaP, GaAs, GaSb, InP, InAs, InSb, AlS, AlP, AlAs, AlSb, PbS, PbSe, Ge and Si, and ternary and quaternary mixtures thereof. Quantum dots have a discrete quantized energy emission spectrum that is directly correlated to its physical size. Quantum dots may act as FRET donors or acceptors.

As used herein, the term “biosensor” refers to a device that uses a biological molecule such as a nucleic acid, protein, carbohydrate or lipid to detect a target biological or chemical substance, condition or reaction and transmits information about the biological or chemical substance, condition or reaction as a signal. The biological molecule may be immobilized on the surface of the biosensor by direct chemical bonding or by indirect bonding. Bonding is often through a polymer film that is applied to the surface of the biosensor. In some embodiments, a biological molecule may be immobilized to the surface of the biosensor by directly incorporating the biological molecule into the polymer film to be applied to the surface.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the disclosures embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In some embodiments, a method of screening an enzyme for one or more characteristics is described. In some embodiments the method comprises the steps of generating a plurality of droplets, wherein at least one droplet comprises an enzyme, a reaction substrate; and one or more redox cofactors, or one or more redox cofactors and one or more detection reagents, wherein the enzyme converts the reaction substrate to a target product. The method may also comprise detecting a signal emitted from the droplet, wherein the signal is a fluorescence signal, a bioluminescence signal or a chemiluminescence signal, wherein the signal is emitted when the redox cofactor is oxidized or reduced when the reaction substrate is converted to the target product by the enzyme, or wherein the signal is emitted when the redox cofactor is oxidized or reduced when the target product is further converted to a subsequent product by the one or more detection reagents in the droplet.

In some embodiments, the method of screening an enzyme for one or more characteristics comprises, in a fluidic device comprising an incubation region comprising a plurality of incubation chambers including a first incubation chamber and a second incubation chamber, wherein the plurality of incubation chambers are configured to allow continuous flow of a plurality of droplets, performing the steps of culturing a cell in the droplet to form a plurality of cells while flowing the cell from the first incubation chamber to the second incubation chamber; continuously flowing the droplet in the incubation region, wherein the droplet comprises, an enzyme, a reaction substrate, and one or more redox cofactors, or one or more redox cofactors and one or more detection reagents, wherein the enzyme converts the reaction substrate to a target product; and generating a signal from the droplet, wherein the signal is a fluorescence signal, a bioluminescence signal or a chemiluminescence signal.

In some embodiments, a fluidic system is described, the fluidic system comprising a plurality of droplets including a first droplet comprising a redox cofactor, wherein a concentration of the redox cofactor in the droplet is greater than or equal to 5 μM, a microfluidic channel containing the droplet, and a droplet-sorting region in fluidic communication with the microfluidic channel, wherein the droplet-sorting region comprises a set of electrodes adjacent to the microfluidic channel.

In some embodiments, the fluidic system comprises a microfluidic channel containing a plurality of droplets, wherein at least one droplet comprises a redox cofactor and a detection reagent. In some embodiments, the detection reagent is configured to react with a redox couple of the redox cofactor to produce a luminescent signal.

In some embodiments, the fluidic system comprises a first reagent chamber configured to contain a first liquid and a plurality of cells, at least one of the cells comprising an enzyme. In some embodiments, the fluidic system comprises a second reagent chamber, wherein the second chamber is configured to contain a reaction substrate; a carrier fluid chamber configured to contain a second liquid immiscible with the first fluid, and in fluidic communication with at least the first reagent chamber; a merging region configured to allow merging of the first and second liquids. In some embodiments, the fluidic system comprises an incubation region in fluidic communication with the merging region, wherein the incubation region comprises: a plurality of incubation chambers including a first incubation chamber and a second incubation chamber in fluidic wherein the first and second incubation chambers are configured to allow continuous flow of droplets between the chambers. In some embodiments, the fluidic system comprises a droplet-sorting region in fluidic communication with the incubation region. In some embodiments, the droplet-sorting region comprises a detection area, a set of electrodes downstream from the detection area configured to sort the plurality of droplets based on detection of a component in the droplet. In some embodiments, the fluidic system comprises a collection channel, and a waste channel. In some embodiments, the fluidic system comprises a bubble trap positioned between the incubation region and the droplet-sorting region. In some embodiments, the fluidic system further comprising a second carrier fluid chamber within the droplet-sorting region.

In some embodiments, the fluidic system comprises a first reagent chamber configured to contain a first liquid and a plurality of cells, at least one of the cells comprising an enzyme, a second reagent chamber, wherein the second chamber is configured to contain a reaction substrate, a carrier fluid chamber configured to contain a second liquid immiscible with the first fluid, and in fluidic communication with at least the first reagent chamber, a merging region configured to allow merging of the first and second liquids, an incubation region in fluidic communication with the merging region, wherein the incubation region comprises a plurality of incubation chambers including a first incubation chamber and a second incubation chamber, a detection area comprising a first detector, wherein the first detector is configured to determine a first luminescent signal of a first wavelength and a second luminescent signal of a second wavelength, wherein the first wavelength and the second wavelength are different, a droplet-sorting region in fluidic communication with the incubation region; a set of electrodes downstream from the detection area configured to sort the plurality of droplets based on detection of a component or components in the droplet; a collection channel, and/or and a waste channel.

In some embodiments, the method further comprises screening the enzyme for the one or more characteristics based on the level of emitted signal detected compared to a reference signal, wherein a change in the level of emitted signal compared to the reference signal indicates that the enzyme has the one or more characteristics.

In some embodiments, the method further comprises isolating the droplets based on the level of emitted signal detected compared to a reference signal.

In some embodiments, the method further comprises sorting the droplets based on the level of emitted signal detected compared to a reference signal.

In some embodiments, the method further comprises culturing cells within the plurality of droplets.

In some embodiments, the method comprises culturing comprises proliferating cells within the plurality of droplets.

In some embodiments, the method further comprises lysing one or more cells. In some embodiments of the method, the detecting step of comprises detecting intracellular or cell surface-displayed enzymes.

In some embodiments, the detecting step comprises detecting extracellular enzymes.

In some embodiments, a lower signal is compared to a reference signal is detected in the absence of NADH in the method.

In some embodiments, a fluorescence signal with an excitation wavelength of 590 nm and an emission wavelength of 620 nm is emitted in the presence of NADH.

In some embodiments, the enzyme is a redox cofactor-dependent enzyme and the signal is emitted when the redox cofactor is oxidized or reduced when the reaction substrate is converted to the target product by the enzyme in the method.

In some embodiments, the enzyme is a redox cofactor-independent enzyme and the signal is emitted when the redox cofactor is oxidized or reduced when the target product is further converted to a subsequent product by the one or more detection reagents in the method.

In some embodiments, generation of the plurality of droplets in step a) further comprises incubating the host cell in each droplet with culture media in the droplet to increase the number of host cells prior to adding the reaction substrate, detection reagents, one or more detection reagents, lysis buffer, redox cofactor or combinations thereof in the method.

In some embodiments, the droplets are isolated by using a method selected from the group consisting of fluorescence-activated droplet sorting (FADS), fluorescence-activated cell sorting (FACS) and magnetic-activated cell sorting (MACS), acoustic control techniques, magnetic control techniques, pneumatic control techniques, thermal control techniques, electric control techniques and combinations thereof in the method.

In some systems and methods, one or more characteristics of the enzyme is selected from the group consisting of enzyme activity, stability, specificity, stereoselectivity, ability to convert a non-natural substrate and combinations thereof.

In some embodiments, the method further comprises the step of sequencing the amino acid sequence, the polynucleotide sequence, or both, of the enzyme in the isolated droplets, optionally wherein the sequenced enzyme is used to generate a library of enzyme variants.

In some embodiments of the method, the enzyme is expressed in a host cell, and wherein the plurality of droplets further comprises the host cell or fragments thereof.

In some embodiments, the method further comprises detecting a luminescent signal upstream the sorting region, optionally comparing the first luminescent signal with the signal emitted from the droplet.

In some embodiments, the method further comprises detecting a first and second luminescent signal, optionally comprising comparing the first and second luminescent signal or a derived signal of the first and second luminescent signal to the signal emitted from the droplet.

In some embodiments of the method, a signal generated upstream a sorting region is transmitted to the sorting region.

In some embodiments, the method further comprises applying a voltage to a set of sorting electrodes.

In some embodiments, the method further comprises screening an enzyme for increasing conversion of its natural substrate, increasing conversion of its non-natural substrate, increasing catalytic activity, increasing stereoselectivity, increasing thermal stability, increasing stability in a predetermined pH range, or a combination thereof.

In some embodiments, the system or the method is described wherein the one or more redox cofactors is selected from the group consisting of NAD⁺, NADH, NADP⁺, NADPH, NAD⁺ and NADP⁺, and NADH and NADPH.

In some embodiments, the system or the method is described wherein each droplet of the plurality of droplets further comprises one or more detection reagents that react with the oxidized or reduced redox cofactor to produce the emitted signal.

In some embodiments, the system or the method is described, wherein the redox cofactor is NADH or NADPH or both, and wherein the one or more detection reagents is selected from the group consisting of: resazurin and diaphorase; proluciferin, luciferase, a reductase, ATP and Mg²⁺; and a quantum dot.

In some embodiments, the system or the method is described wherein the redox cofactor is NAD⁺ or NADP⁺ or both, and wherein the one or more detection reagents is selected from the group consisting of: a quantum dot; and a protein biosensor.

In some embodiments, the system or the method is described wherein the quantum dot is a CdSe/CdS/CdZnS/ZnS quantum dot.

In some embodiments, the system or the method is described wherein the quantum dot is a CdSe/CdS/CdZnS/ZnS quantum dot coated with carboxylic acid and bovine serum albumin and Nile blue dye.

In some embodiments, the system or the method is described wherein the quantum dot is water soluble.

In some embodiments, the system or the method is described wherein the quantum dot is a CdSe/CdS/CdZnS/ZnS quantum dot coated with 3-mercaptopropionic acid and linked to a NAD+ binding domain.

In some embodiments, the system or the method is described wherein the NAD⁺ binding domain is a thiolated DBC1 protein sensor.

In some embodiments, the system or the method is described wherein compared to a reference signal, a lower fluorescence signal with excitation wavelength of 538 nm and emission wavelength of 595 nm is emitted in the presence of NAD⁺.

In some embodiments, the system or the method is described wherein the enzyme is a redox cofactor-dependent enzyme or a redox cofactor-independent enzyme.

In some embodiments, the system or the method is described wherein the host cell is selected from the group consisting of a mammalian cell, a plant cell, a bacterial cell, a fungal cell, a yeast cell, a protozoan cell, an algal cell and an archaeal cell.

In some embodiments, the system or the method is described wherein the at least one or more of the plurality of droplets further comprises a lysis buffer.

In some embodiments, the system or the method is described

The system or method of any one of the preceding claims, wherein the plurality of droplets are selected from the group consisting of water-in-oil droplets, water-in-oil-in-water droplets, oil-in-water droplets and hydrogel droplets.

In some embodiments, the system or the method is described wherein the plurality of droplets have a diameter of between about 10 μm and 100 μm.

In some embodiments, the system or the method is described wherein the plurality of droplets have a diameter of about 30 μm.

In some embodiments, the system or the method is described wherein the enzyme is selected from the group consisting of an intracellular enzyme, a surface displayed enzyme, or an extracellular enzyme.

In some embodiments, the system or the method is described wherein the enzyme is selected from the group consisting of secondary alcohol dehydrogenase from Candida parapsilosis (CpSADH), epoxide hydrolase from Sphingomonas sp. HXN-200 (SpEH), styrene oxide isomerase (SOI), P450 monooxygenase (P450pyr), amine dehydrogenase from Rodococcus (AmDH), and butanediol dehydrogenase (BDHA).

In some embodiments, the system or the method is described wherein the enzyme is an enzyme variant selected from a library of enzyme variants of a single enzyme, or wherein the enzyme is selected from a group of different enzymes.

In some embodiments, the system or the method is described wherein the cofactor is an exogenous cofactor.

In some embodiments, the system or the method is described that further comprises one or more droplets each containing at least one cell comprising an endogenous cofactor concentration, and wherein an exogenous cofactor concentration is greater than an endogenous cofactor concentration.

In some embodiments, the system or the method described that further comprises a detector positioned upstream of a sorting region.

In some embodiments, the system or the method is described that further comprises a bubble trap, the bubble trap optionally comprising a vacuum port.

Other embodiments are within the following claims and non-limiting examples. In addition, where features or aspects of the disclosure are described in terms of Markush groups, those skilled in the art will recognize that the invention is also thereby described in terms of any individual member or subgroup of members of the Markush group.

The following examples are intended to illustrate certain embodiments of the present invention, but do not exemplify the full scope of the invention.

### EXAMPLE 1

The following example describes some general materials and methods for enzyme screening and sorting.

### Materials and Methods

### Mutant Library Construction

Mutant libraries were constructed by site-saturation mutagenesis at 3-4 selected amino acid sites. PCR was performed with the corresponding primers containing degenerate NDT or NNK codons (or combination) at the desired sites. The PCR product was digested with the corresponding restriction enzymes, cloned to an expression vector and transformed in E. coli (BL21, DE3) competent cells. The plasmid of each library was extracted and sequenced to confirm the successful introduction of degenerated codons at the corresponding target sites.

### Cell Culture

E. coli expressing the desired enzyme was cultivated in 5 mL of LB media for 12 h at 22° C. and 250 rpm. IPTG (final concentration 0.5 mM) was added at 2 h to induce the enzyme expression. Cells were harvested by centrifuging at 4,000 g for 2 min, washed twice with phosphate buffer (50 mM, pH 7.4), and resuspended in phosphate buffer (50 mM, pH 7.4) to the final density of 2×10⁷ cells/mL (OD₆₀₀=0.015).

### Cell Encapsulation

A 3-channel flow-focusing microfluidic chip was used to produce 30 μm diameter droplets at a speed of at least 1000 droplets/second. During the operation, two aqueous samples were mixed at 1:1 ratio and a flow rate of 1 μL/min, and fluorinated oil HFE-7500 (containing surfactant) was supplied at a flow rate of 3 μL/min.

### Parameters Used for Droplet Sorting

Droplets were collected and introduced into a sorting chip at a speed of 100-1000 droplets/second. The droplets flowed in a single file, and the interval between each droplet was enlarged by supplying a spacing oil at 5 μL/min. The fluorescence signal of each droplet was registered when passing through a 200 μm field of view in a microscope with a photomultiplier (PMT) detector. The voltage output of the PMT was processed by a DAQ data acquisition unit (National Instrument, USA) and recorded in MATLAB software. In parallel, the same PMT output was digitized by an Arduino Due microcontroller at a sample rate of 500 kHz. Once the signal was in the desired range compared to the pre-set sorting threshold, a sorting pulse was sent to the downstream electronics, amplified and sent to the sorting electrodes to trigger the droplet sorting. Liquid flow was delivered by high-accuracy syringe pumps (Pump 11 Pico Plus Elite, Harvard Apparatus) through a glass syringe (Hamilton) or plastic syringe (Terumo) and PTFE tubing.

### Protocol for Droplet Merging

After connecting the tubing containing the solutions into the corresponding inlets on the chip, the flowrate was set as follows: spacing oil at 2 μL/min, droplets at 0.7 μL/min, and Merging solution at 0.2 μL/min.

the function generator was then used at 20 kHz frequency and 1 V amplitude and connected to amplifier to generate the 100V pulses. the Ground and high voltage outlets from amplifier were connected to corresponding electrodes on the chip. After reaching a steady flow in the channels, the amplifier turned on and merging performed.

### EXAMPLE 2

The following example describes the synthesis of NADH and NAD⁺ sensing quantum dots functionalized with Nile blue. NADH sensing QDs synthesis

(1) Synthesis of CdSe Core Nanocrystals. Highly fluorescent CdSe nanocrystals were made by a procedure modified from those reported previously. For a typical reaction, the mixture of 0.2 mmol of CdO, 0.8 mmol of stearic acid, and 2 g of ODE in a 25 mL three-neck flask was heated to about 200° C. to obtain a colorless clear solution. After this solution was cooled to room temperature, ODA (1.5 g) and TOPO (0.5 g) were added into the flask. Under nitrogen flow, this system was reheated to 280° C. At this temperature, a selenium solution made by dissolving 2 mmol of Se in 1.37 g ODE was quickly injected. The growth temperature was then reduced to 260° C. The nanocrystals were purified by precipitating in methanol. At the determined reaction time, it generates CdSe nanocrystals of about 3.2 nm in size with the first absorption peak around 580 nm.

2) Synthesis of CdSe/CdS/CdZnS/ZnS. CdSe nanocrystals (3.2 nm in diameter, 1.07×10⁷ mol of particles) dissolved in chloroform were mixed with 1.5 g ODA and 3.5 g of ODE in a 25 mL three-neck flask. The flask was heated up to 180° C. for 1 h to remove chloroform and residual air from the system. Subsequently, the system was switched to nitrogen flow and the reaction mixture was further heated to 240° C. for the serial injection. The first injection was 0.38 mL of Cd injection solution (0.01 M), and the amounts of subsequent injection solutions were calculated using the reported method. The reaction was terminated by allowing the reaction mixture to cool. The final product was diluted by chloroform and precipitated by adding acetone into the chloroform solution. At the determined reaction time, this reaction generates CdSe/CdS/CdZnS/ZnS quantum dot with the first absorption peak around 590 nm and emission peak around 616-620 nm.

(3) Water solubilization of quantum dots. 3-mercaptopropionic acid (MPA) (400 μL, 4.59 mmol) was added to quantum dots in chloroform with an optical density of 1.5, and the solution was shaken. The chloroform solution turned turbid. The quantum dots were then dissolved in water.

(4) Preparation of BSA coated quantum dots. The MPA-capped quantum dots were mixed with a 1000-fold excess of bovine serum albumin (BSA) in 10 mM HEPES buffer (pH 7.4) containing 10 mM 1-ethyl-3-[3-dimethylaminopropyl] carodiimide hydrochloride (EDC), and the mixture was shaken for 24 h at 4° C. The BSA coated quantum dots were obtained by ultrafiltration.

(5) Preparation of Nile blue capped quantum dots. To the quantum dots particle solution was added BS3 (bis[sulfosuccinimidyl]suberate) stock solution (50 mL, 1 mg/mL in 10 mM HEPES buffer, pH 8), and the mixture was shaken for 15 min. The resulting quantum dots was mixed with a 50-fold excess of stock solution of nile blue (1 mg/mL in 3:2 ethanol/water), and the mixture was shaken for 16 h. Excess of Nile blue was removed by dialysis against 10 mM HEPES buffer pH 8 (MW cutoff 10 k.)

### NAD⁺ Sensing Quantum Dots Synthesis

Detailed procedures of synthesis of quantum dot-based NAD⁺ biosensor are described below.

1) Synthesis of cadmium selenide (CdSe) Core Nanocrystals. Highly fluorescent CdSe nanocrystals were made by a procedure modified from those reported previously. For a typical reaction, the mixture of 0.2 mmol of cadmium oxide (CdO), 0.8 mmol of stearic acid, and 2 g of octadecene (ODE) in a 25 mL three-neck flask was heated to about 200° C. to obtain a colorless clear solution. After this solution was cooled to room temperature, octadecylamine (ODA) (1.5 g) and Trioctylphosphine oxide (TOPO) (0.5 g) were added into the flask. Under nitrogen flow, this system was reheated to 280° C. At this temperature, a selenium solution made by dissolving 2 mmol of Se in 1.37 g ODE was quickly injected. The growth temperature was then reduced to 260° C. The nanocrystals were purified by precipitating in methanol. At the determined reaction time, it generates CdSe nanocrystals of about 2.3 nm in size with the first absorption peak around 528 nm.

(2) Synthesis of CdSe-core CdS(2Ls)/Cd_{0.5}Zn_{0.5}S(3 L)/ZnS(2 Ls) multi shell quantum dots (QDs) were synthesized. CdSe nanocrystals (2.3 nm in diameter, 1.02×10⁷ mol of particles) dissolved in chloroform were mixed with 1.5 g ODA and 3.5 g of ODE in a 25 mL three-neck flask. The flask was heated up to 180° C. for 1 h to remove chloroform and residual air from the system. Subsequently, the system was switched to nitrogen flow and the reaction mixture was further heated to 240° C. for the serial injection. The first injection was 0.38 mL of Cd injection solution (0.01 M), and the amounts of subsequent injection solutions were calculated using the reported method. The reaction was terminated by allowing the reaction mixture to cool. The final product was diluted by chloroform and precipitated by adding acetone into the chloroform solution. At the determined reaction time, this reaction generates CdSe/CdS/CdZnS/ZnS quantum dot with the first absorption peak around 538 nm and emission peak around 595 nm.

(3) Preparation of MPA capped QDs. The above QDs were precipitated from the chloroform solution by addition of 4 mL of methanol to 1 mL of QDs in chloroform, followed by centrifugation for 10 minutes at 8000 rpm. The resulting precipitate was re-dissolved in 1 mL chloroform, to which was added 200 μL of 3-Mercaptopropionic acid (MPA). After the addition of 4 mL of 1 mM NAOH solution, the particles were transferred to water phase. The water phase was collected by centrifugation for 1 min. The excess MPA was removed by two successive precipitation steps of QDs, using saturated NaCl and methanol followed by centrifugation. The resulting MPA capped QDs were re-dissolved in 1 mL of 10 mM HEPES buffer (pH 7.4).

(4) Preparation of thiolated DBC1 protein (deleted in breast cancer 1). DBC1 protein was prepared by treated protein with 2-iminothiolane to introduce sulfhydryl residues onto a sulfydril:protein ratio of ˜20. Five mg of DBC1 dissolved in 1 mL of PBS (pH 7.4) was allowed to react with 2 mg of 2-iminothilane.HCl and kept at 4° C. for 2 h with vortex. The resulting solution was filtered through a size-exclusion centrifugal filter device (Micron YM-30, Millipore).

(5) Preparation of DBC1 protein coated QDs. The MPA capped QDs were incubated with thiolated DBC1 protein in HEPES buffer (pH 7.4) for 5 days at 4° C.

### EXAMPLE 3

The following example describes a fluidic system for high-throughput screening and sorting of cells containing target enzymes.

### Microfluidic Systems for High-Throughput Screening of Intracellular or Surface Displayed Enzymes

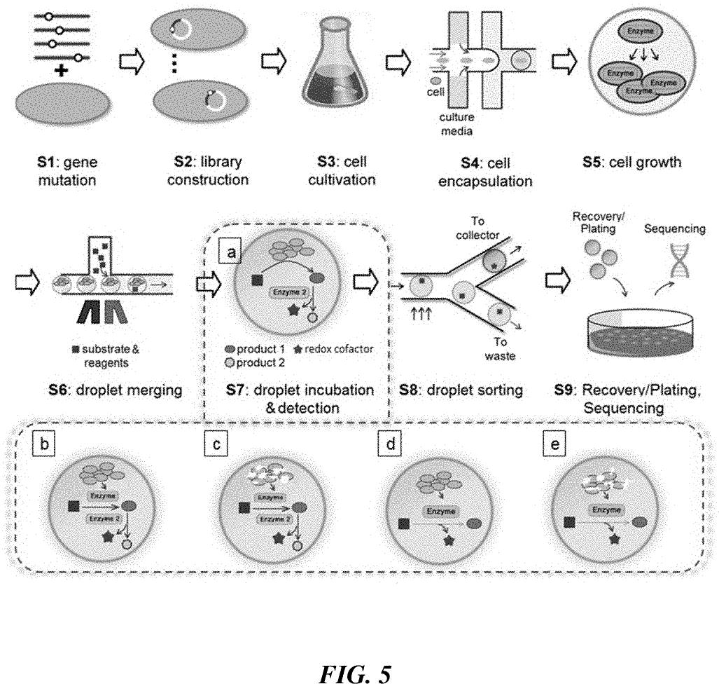

This system incorporated various components for the screening of enzymes and could be used for both cofactor-dependent and cofactor independent reactions with intracellular or surface displayed enzymes as shown in FIG. 5. A mutant library of a target enzyme (10⁴-10⁷ variants) was prepared and transferred into the host microorganism (cell), where each cell expressed only a single enzyme variant. The cells of the library were encapsulated with single cell inside the water-in-oil microdroplets (10-100 μm) containing the reaction substrate, NAD⁺ or NADH, and if required, the detection enzyme and/or lysis buffer. This system could be applied to all forms of intracellular enzymes with whole-cells or lysed cells as well as surface displayed enzymes with whole-cells.

In FIGS. 4A-4C, the detection of cofactor-independent enzymatic reactions was achieved via conversion of the target biotransformation product with another enzymatic reaction to produce the oxidized or reduced cofactor for the highly sensitive and accurate detection of such cofactor and the target reaction thereof. In FIGS. 4A-4E, the highly sensitive and accurate detection of the target reaction was achieved by bioluminescence or fluorescence detection of reduced or oxidized cofactor produced from the target enzymatic reactions by cofactor-dependent enzymes.

The system of FIG. 4A shows the screening of a cofactor-dependent or cofactor-independent intracellular enzyme in a whole cell. After encapsulation of a single cell expressing a single enzyme variant, the droplets that readily contain the reaction substrate, the detection enzyme (enzyme 2), and oxidized or reduced cofactor were incubated. The substrate which diffused into the cell was converted to the product by the intracellular enzyme variant. The resulting product which diffused out of the cell was further converted by the detection enzyme (enzyme 2) to generate the reduced or oxidized cofactor that could be quantitatively detected. Sorting of the droplets with the desired signal level indicated the variants with the desired catalytic performance.

The system in FIG. 4B shows the screening of a surface displayed and cofactor-independent enzyme. After encapsulation of a single cell expressing a single enzyme variant, the droplets that readily contained the reaction substrate, the detection enzyme (enzyme 2), and oxidized or reduced cofactor were incubated. Biotransformation was conducted outside the cell by the surface displayed enzyme and this was followed by highly sensitive and accurate detection via conversion of the target biotransformation product with another enzymatic reaction to produce the reduced or oxidized cofactor and sorting of these droplets.

The system in FIG. 4C shows the screening of NAD(P)⁺- or NAD(P)H-independent intracellular enzyme with lysed cells. After encapsulation of a single cell expressing a single enzyme variant, the droplets that readily contained the reaction substrate, lysis buffer, the detection enzyme (enzyme 2), and oxidized or reduced cofactor were incubated. The target biotransformation was conducted by the intracellular enzymes which were released from the lysed cell into the droplet. This was followed by highly sensitive and accurate detection via conversion of the target biotransformation product with another enzymatic reaction to produce the reduced or oxidized cofactor and sorting of these droplets.

The system in FIG. 4D shows the screening of a surface displayed and cofactor-dependent enzyme. After encapsulation of a single cell expressing a single enzyme variant, the droplets that readily contained the reaction substrate and oxidized or reduced cofactor were incubated. Biotransformation was conducted outside the cell by the surface displayed enzyme. This was followed by highly sensitive and accurate detection of the reduced or oxidized cofactor and sorting of these droplets.

The system in FIG. 4E illustrates the screening of cofactor-dependent intracellular enzyme with lysed cells. After encapsulation of the single cell expressing a single enzyme variant, the incubation of the droplets that readily contained the reaction substrate, lysis buffer, and a suitable cofactor was conducted. The intracellular enzymes were released from the lysed cell into the droplet. The target biotransformation was performed by the enzyme and this was followed by highly sensitive and accurate detection of oxidized or reduced cofactor and sorting of these droplets.

Microfluidics Systems for High-Throughput Screening of Extracellular Enzymes as Well as Intracellular and Surface Displayed Enzymes with Low Activity or Low Expression Level

This system incorporated various components with cell growth in the droplet and droplet merging for the screening of the both cofactor-dependent or cofactor-independent extracellular enzymes as well as intracellular and surface displayed enzymes with low activity or low expression level as illustrated in FIG. 5. In this system, the single E. coli cell was first encapsulated with culture medium inside the droplets. With incubation at 20-40° C. and optimal shaking conditions, the single cell inside the droplet was allowed to reproduce. The reaction substrate, a suitable cofactor, and if required the detection enzyme and/or lysis buffer, was then introduced into the droplet through droplet merging. This system could be applied to all intracellular enzymes with whole-cells or lysed cells with low activity or low expression level as well as extracellular enzymes.

In FIGS. 5A-5C, the detection of cofactor-independent enzymatic reactions was achieved via conversion of the target biotransformation product with another enzymatic reaction to produce oxidized or reduced cofactor for the highly sensitive and accurate detection. In FIGS. 5D-5E, the highly sensitive and accurate detection of the target reaction were achieved by bioluminescence or fluorescence detection of NAD(P)H or NAD(P)⁺, produced from the target enzymatic reactions by the cofactor-dependent enzymes.

The system of FIG. 5A shows the screening of cofactor-dependent or independent intracellular enzyme with low activity or low expression level in whole-cell. After encapsulation of a single cell expressing a single enzyme variant in the culture medium, the cell was grown in the droplet and this was followed by the addition of the reaction substrate, the detection enzyme (enzyme 2), and a suitable cofactor to the droplet through droplet merging. The substrate which diffused into the cell was converted to the product by the intracellular enzyme variant. The resulting product which diffused out of the cell was further converted by the detection enzyme (enzyme 2) to generate oxidized or reduced cofactor for the highly sensitive and accurate detection. Sorting of the droplets with high signal indicated the variants with the desired catalytic performance.

The system in FIG. 5B shows the screening of an extracellular cofactor-independent enzyme. After encapsulation of a single cell expressing a single enzyme variant in the culture medium, the cell was grown in the droplet and this was followed by the addition of the reaction substrate, the detection enzyme (enzyme 2), and a suitable cofactor to the droplet through droplet merging. The droplets were then incubated, and the biotransformation was conducted outside the cell by the extracellular enzyme. This was followed by highly sensitive and accurate detection via conversion of the target biotransformation product with another enzymatic reaction to produce oxidized or reduced cofactor and sorting of these droplets.

The system in FIG. 5C shows the screening of an intracellular cofactor-independent enzyme with low activity or low expression level using lysed cell. After encapsulation of a single cell with a single enzyme variant in the culture medium, the cell was grown in the droplet and this was followed by the addition of the reaction substrate, the detection enzyme (enzyme 2), lysis buffer, and a suitable cofactor to the droplet through droplet merging. The droplets were then incubated. The target biotransformation was performed by the intracellular enzymes which were released from the lysed cell into the droplet. This was followed by highly sensitive and accurate detection via conversion of the target biotransformation product with another enzymatic reaction to produce oxidized or reduced cofactor and sorting of these droplets.

The system in FIG. 5D shows the screening of an extracellular cofactor-dependent enzyme. After encapsulation of a single cell with a single enzyme variant in the culture medium, the cell was grown in the droplet and this was followed by addition of the reaction substrate and a suitable cofactor to the droplet through droplet merging. These droplets were then incubated. The biotransformation was conducted outside the cell by the extracellular enzyme. This was followed by highly sensitive and accurate detection of oxidized or reduced cofactor and sorting of these droplets.

The system in FIG. 5E shows the screening of a cofactor-dependent enzyme with low activity or low expression level using lysed cell. After encapsulation of a single cell with a single enzyme variant in the culture medium, the cell was grown in the droplet and this was followed by the addition of the reaction substrate, lysis buffer, and a suitable cofactor to the droplet through droplet merging. These droplets were then incubated. The target biotransformation was performed by the intracellular enzymes which were released from the lysed cell into the droplet. This was followed by highly sensitive and accurate detection of oxidized or reduced cofactor and sorting of these droplets.

### Generic Assays for Highly Sensitive and Accurate Detection of NAD(P)⁺ and NAD(P)H in Microfluidics

To detect the NAD(P)⁺ or NAD(P)H that are produced from enzymatic reactions in microdroplets, various high-throughput assays for microfluidics with high sensitivity and different applications were developed. The assays are summarized in Table 1.

TABLE 1
High-throughput assays for detection of NAD(P)⁺ or NAD(P)H in
microfluidics.

| | Detection of | Detection of |
|---|---|---|
| Type of assay | NAD(P)⁺ | NAD(P)H |
| Direct fluorescence detection | | ✓ |
| Fluorescence detection by | | ✓ |
| formation of resorufin | | |
| Bioluminescence detection | | ✓ |
| Detection with quantum dots | ✓ | ✓ |
| through FRET effect | | |
| Detection with protein | ✓ | |
| biosensor | | |

### Direct Fluorescence Detection of NAD(P)H

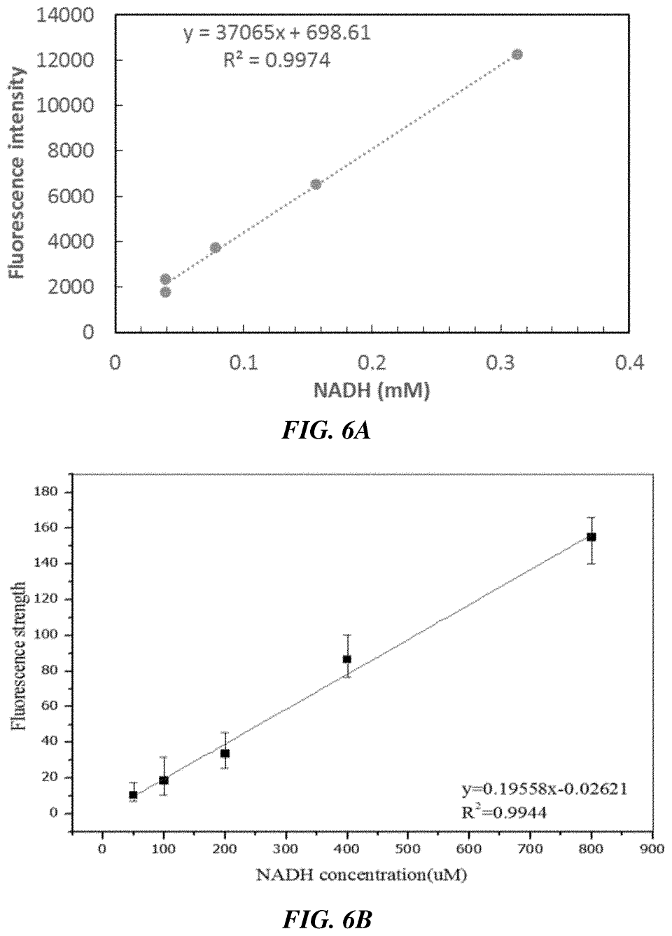

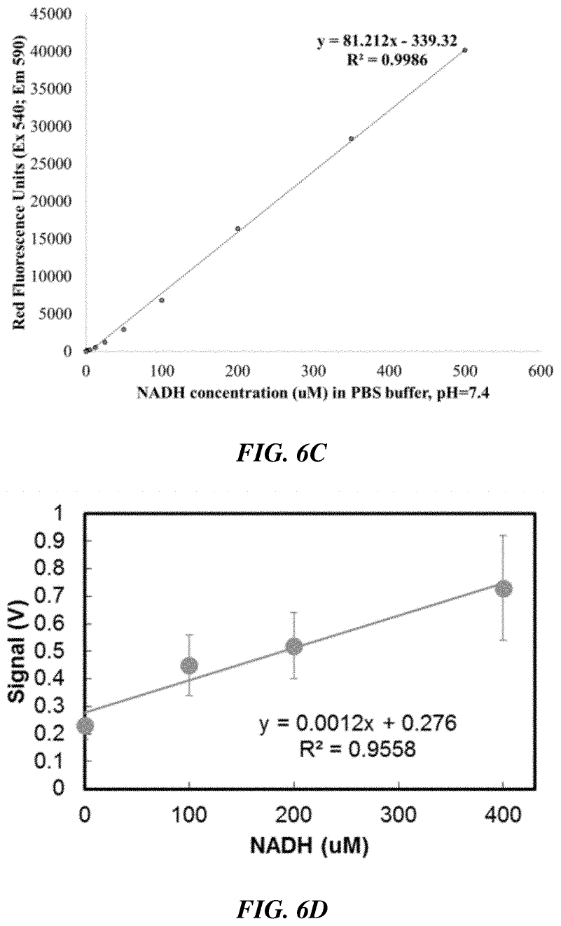

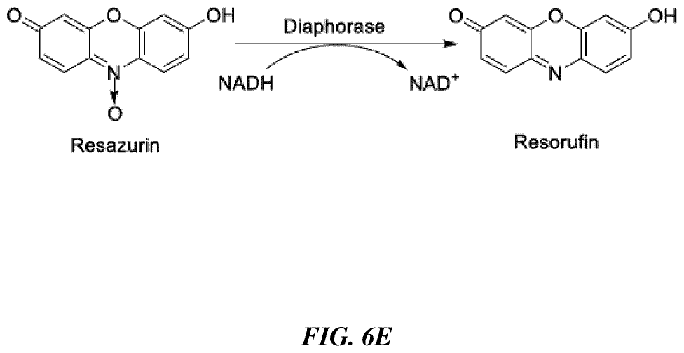

NADH is a fluorescent molecule with 0.1 quantum efficiency. When excited at 340 nm, NADH can be also detected with its own intrinsic fluorescence at 450 nm. In comparison, NAD⁺ has distinctive spectrum feature and is not detectable under the same conditions. The results of the direct detection of NADH fluorescence (with 340 nm excitation and 450 nm emission) in microtiter plate is shown in FIG. 6A. The intensity of the fluorescence signal showed very good linear relationship to the NADH concentration in the range of 40-300 μM.

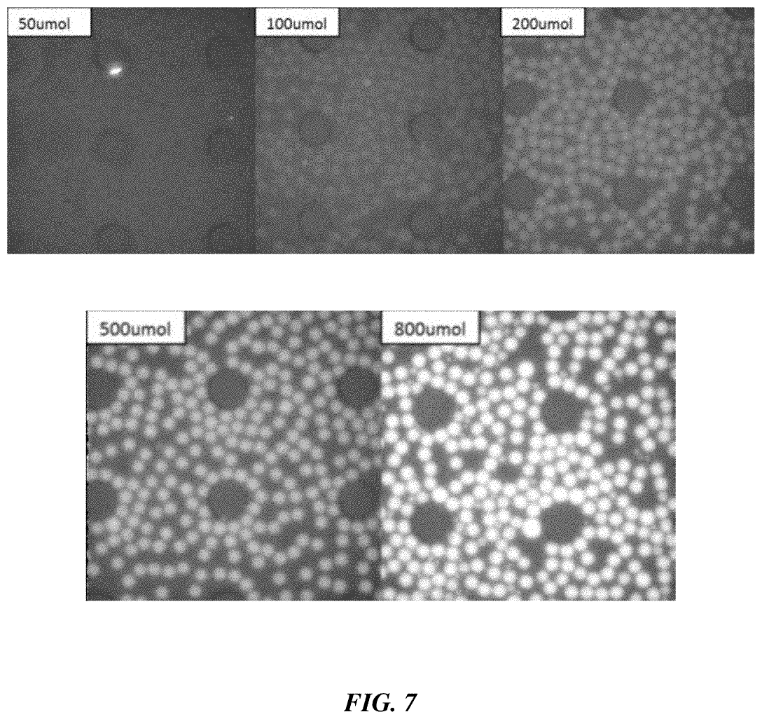

The detection was further validated in microfluidic droplets with diameter of 30 μm and various NADH concentration (50, 100, 200, 400 and 800 μM). The fluorescence signal from NADH in droplets could be captured with a CMOS camera, and the mean fluorescence intensity was calculated through image analysis with ImageJ software (FIG. 6B). The fluorescence intensity linearly correlated to the NADH concentration, with signal to noise ratio (S/N) of 8.1±1.1. In comparison, the detection with PMT gave a much lower S/N of 0.5±0.2, thus the direct fluorescence detection was used to develop image-based sorting system that requires to use CMOS camera (FIG. 7).

### Fluorescence Detection of NAD(P)H by Formation of Resorufin

To develop a more sensitive assay, a cascade reaction that led to the formation of a red fluorescent molecule was incorporated. As shown in FIG. 6E, NAD(P)H rapidly reacts with a weak fluorophore resazurin in the presence of diaphorase, to give a strong red fluorescent product resorufin, which can be detected at 590 nm with an excitation at 540 nm. This coupled enzymatic reaction linked the formation of highly fluorescent resorufin to the catalytic activity of enzyme of interest. In the microtiter plate test, the fluorescence signal of resorufin showed very good linear relationship to the NAD(P)H concentration in the range of 10-500 μM, and the detection limit is 10 μM (FIG. 6C). The resazurin-based NAD(P)H detection was further validated in microfluidics (FIG. 6D). The fluorescence signal from resazurin of the droplet with different NAD(P)H concentration was determined with a photomultiplier tube (PMT) detector. The result showed a linear relationship between the fluorescence intensity and NAD(P)H concentration from 100 to 400 μM. In the presence of 100 μM of NAD(P)H, the signal to background noise ratio is 2.5±0.7. This method was highly specific to NAD(P)H and the presence of NAD(P)⁺ does not affect the fluorescence signal.

### Bioluminescence Detection of NADH

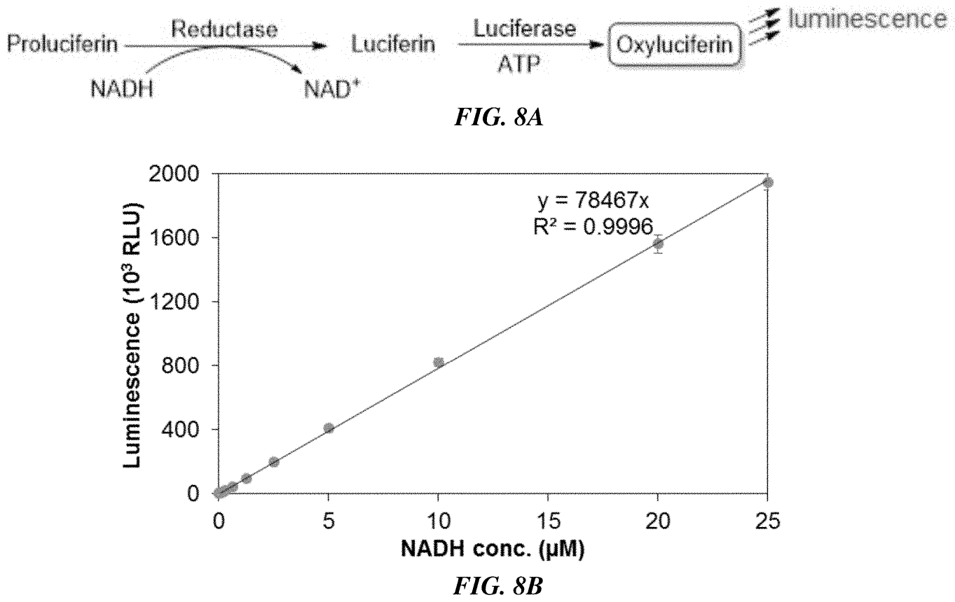

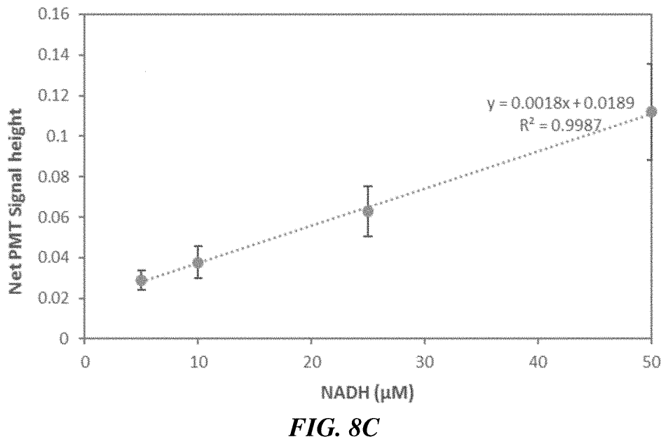

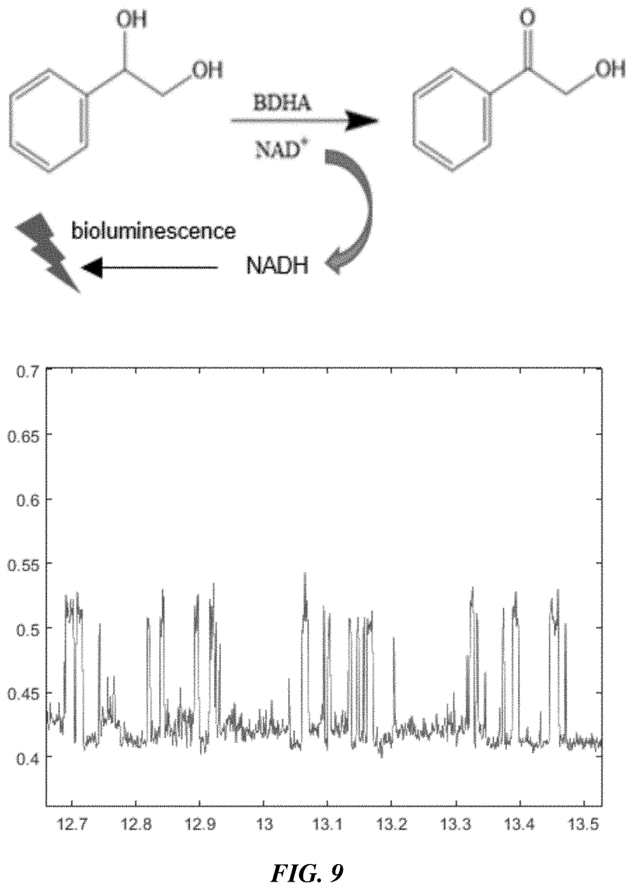

The use of bioluminescent-based biosensor for detecting NADH provided more sensitive detection. In this example, the detection of NADH was performed with commercially available bioluminescence NADH detection kit (NADH-Glo, Promega), which contains luciferase (recombinant beetle luciferase), ATP, and Mg²⁺ for luciferase activity, and proluciferin that could be reduced to D-luciferin by a reductase in presence of NADH, and thus, generate the luminescence signal (FIG. 8A).

The method was examined in microtiter plate and showed a much lower detection limits (0.1 μM) compared with fluorescence-based methods. The linear range for the detection is from 0.1 μM to 25 μM (FIG. 8B). To detect the NADH in microfluidic droplets with the bioluminescent assay, a PMT detector was used to capture the bioluminescent light emission in the presence of NADH. The results showed a detection limit as low as 5 μM NADH, with nearly linear response over the range of 5-50 μM (FIG. 8C). The method did not respond to the presence of NAD⁺.

Detection of NADH produced from BDHA-catalyzed reaction of 1-phenyl-1,2-ethanediol (PED) using bioluminescent assay in the microfluidics system was also examined. The reaction of PED catalyzed by BDHA in KP buffer (pH 7.5, 50 mM) was conducted in microdroplets. The final concentrations of the reagents in the droplet were 100 μM PED, 0.8 mg/mL BDHA, and 0.5 mM NAD⁺. The solution containing enzyme and substrate as well as a solution of bioluminescent assay reagent containing NAD⁺ in 1:1 v/v were injected from two separate inlets and mixed once the droplets formed. The droplets were analyzed by PMT detector after 2 h incubation in the room temperature (FIG. 9).

The quantification of NADH concentrations obtained from analysis of several droplets' peaks using our calibration curve gave concentration of 21±4 μM. This result was consistent with the results from 96-well plate experiment at 2 h (23.5 μM).

Detection of NADH with Quantum Dot Through FRET Effect

Without wishing to be bound by any particular theory, it is believed fluorescence resonance energy transfer (FRET) is a process by which energy transfers between two light-sensitive molecules such as chromophores and fluorophores. A donor fluorophore, initially in its electronic excited state, may transfer energy to an acceptor fluorophore through non-radiative dipole-dipole coupling, then the fluorescence emission of the FRET system can be different depending on the distance and status of the two fluorophores.

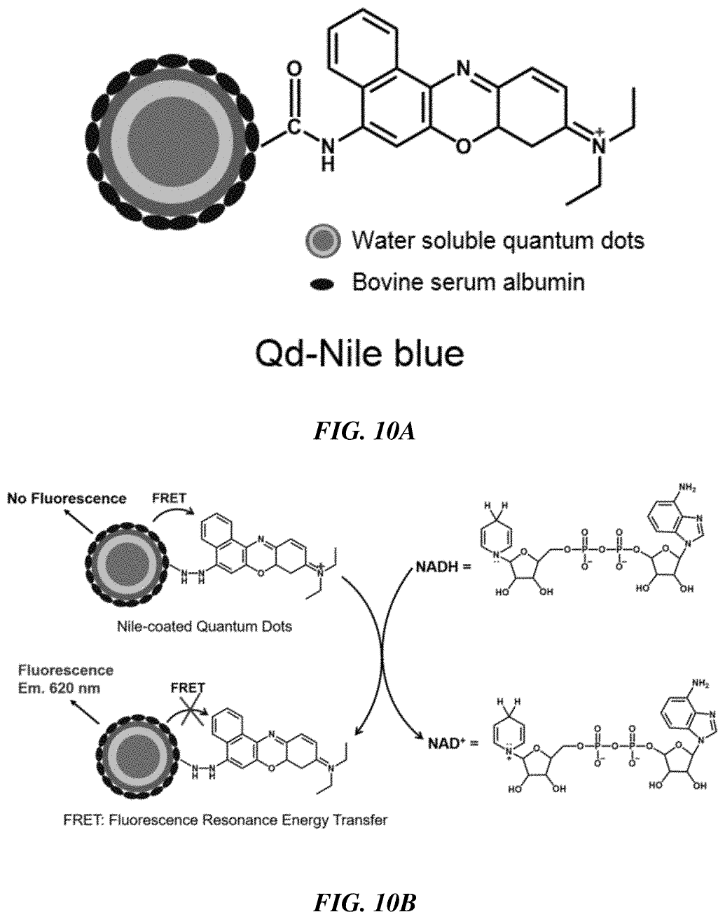

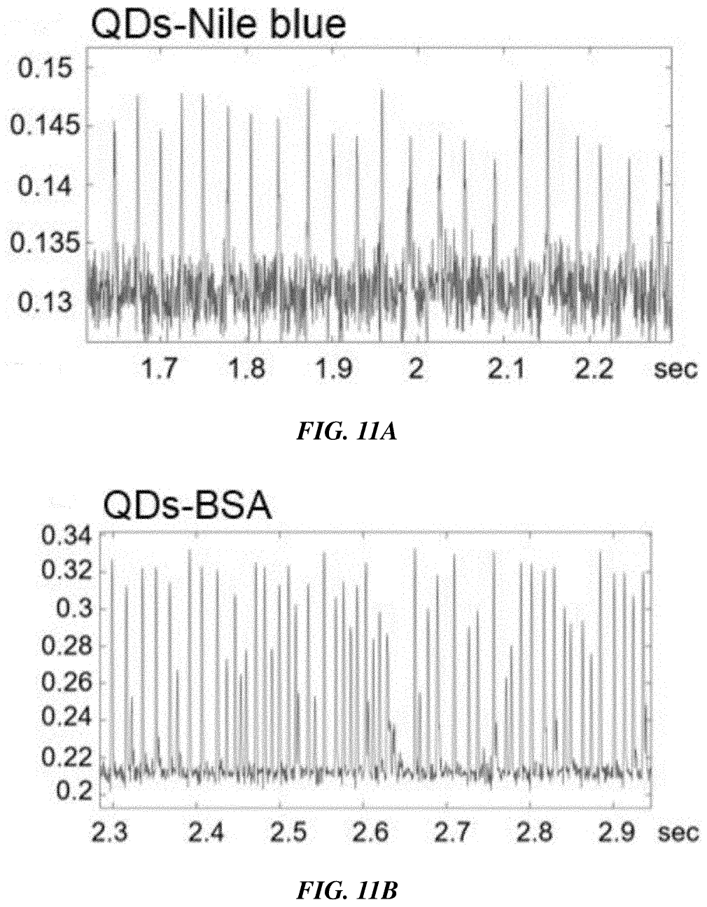

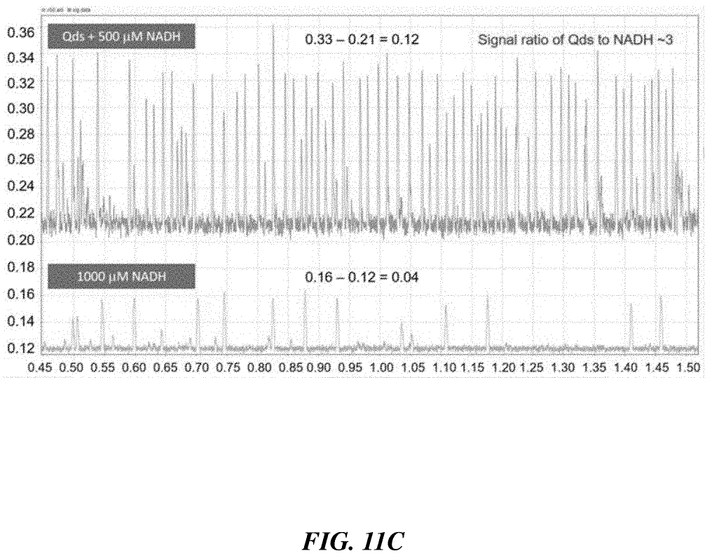

A quantum dot-based NADH sensor with FRET effect was developed (FIG. 10). The highly fluorescent CdSe quantum dot was coated with CdSe/CdS/CdZn nanocrystals, carboxylic acid and bovine serum albumin to give the quantum dot core with an excitation at 590 nm and bright fluorescence at 616-620 nm. For the NADH detection, Nile blue was conjugated onto the surface of quantum dots, which diminished the fluorescence via FRET effect. In the presence of NADH, Nile blue was reduced and the fluorescence of the quantum dot was restored. The strength of the fluorescence was closely related to the concentration of NADH, thus, allowed quantitative detection. The detection of NADH with the quantum dot was tested in microfluidic system (FIG. 11). NADH could be readily detected at NADH concentration>500 μM with a signal to noise ratio of 3.

### Generic Assays for Highly Sensitive and Accurate Detection of NAD⁺ in Microfluidics

Detection of NAD⁺ with Protein Biosensor

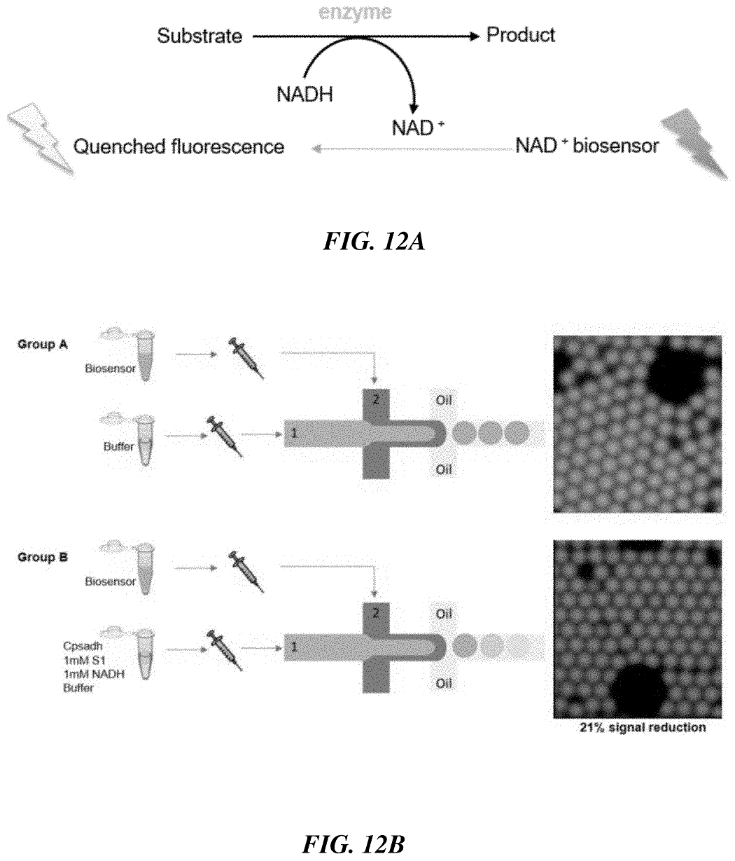

A fluorescent protein-based NAD⁺ biosensor was developed for quantitative detection of NAD⁺. The biosensor consisted of a fluorescent core domain (excitation at 488 nm, emission at 520 nm) and a bipartite NAD⁺ binding domain. In this setup, the fluorescence from the core domain was quenched by NAD⁺ binding corresponding to the concentration of the bound NAD⁺ (FIG. 12A). The biosensor fluorescence intensity and NAD⁺ concentration were negatively correlated in the range of 1 to 1000 μM of NAD⁺. The NAD⁺ detection with the protein biosensor was examined at various NAD⁺ concentration in microfluidic droplets. A nearly-linear correlation between the biosensor fluorescence intensity and NAD⁺ concentration was shown at range of 50 μM to 1000 μM of NAD⁺. The detection of the NAD⁺ using biosensor in microdroplets was demonstrated with 1 mM of NAD⁺. Those droplets that contained the NAD⁺ showed 21% lower fluorescence signal due to the quenching mechanism (FIG. 12B).

Detection of NAD⁺ with Quantum Dot Through FRET Effect

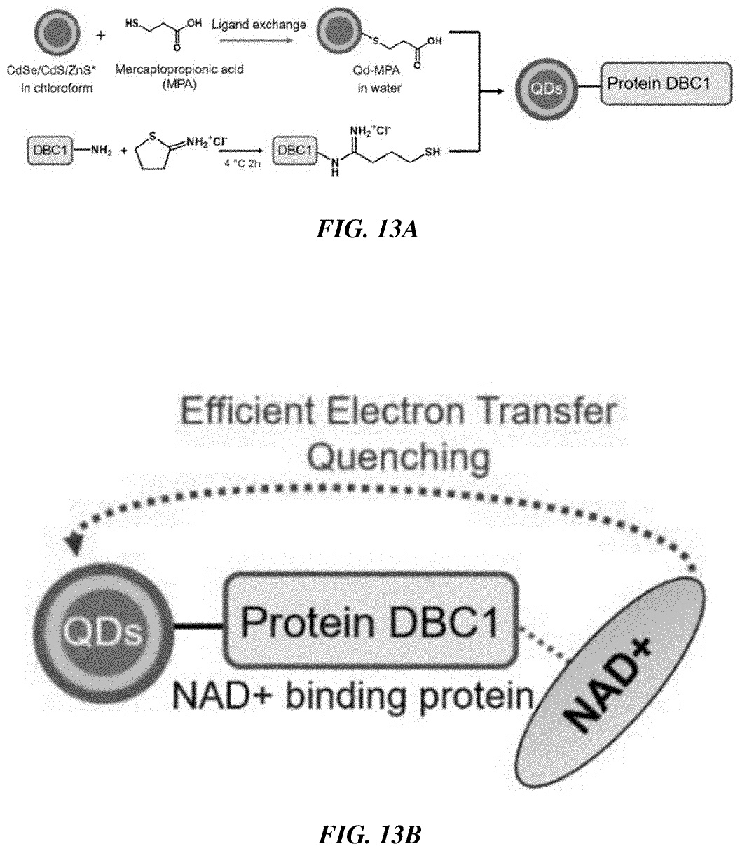

Another example for NAD⁺ sensing was developed with a quantum dot fluorescence sensor through FRET effect (FIG. 13A). The highly fluorescent CdSe quantum dot core was coated with 3-mercaptopropionic acid and linked to a NAD⁺ binding domain (thiolated DBC1 protein sensor). The protein sensor was reported to specifically bind NAD⁺. When such binding occurs, the fluorescence of quantum dot was effectively quenched (FIG. 13B). Consequently, the fluorescence signal of the quantum dot sensor was negatively correlated to NAD+ concentration. This allowed accurate determination of NAD⁺ based on the change of fluorescence signal.

The performance of the quantum dot NAD⁺ sensor was explored. The quantitative NAD⁺ quenching was further examined and validated with an excitation at 538 nm and the fluorescence emission is detected at 595 nm. As concentration of NAD⁺ increased, the fluorescence of the sensor was quenched at a very low NAD⁺ concentration. The sensor enabled the detection of NAD⁺ with a detection limit of 30 μM with a sensor concentration of 1.5 μM with a fixed incubation time of 20 minutes. When the NAD⁺ concentration reached 250 μM and above, more than 90% fluorescence signal is quenched. The quantum dot sensor was highly specific to NAD⁺. The presence of NAD(P)H did not affect the fluorescence signal.

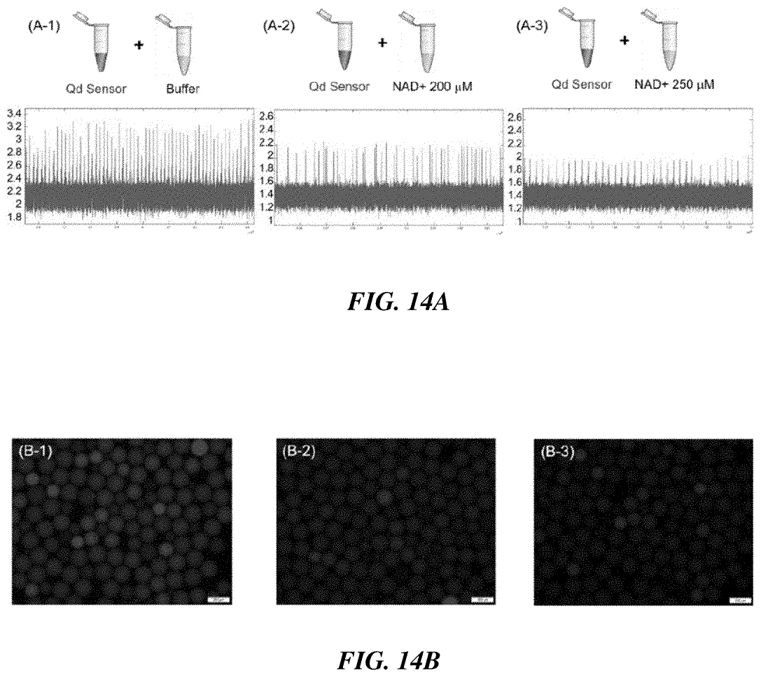

For the NAD⁺ detection in microfluidics, the fluorescence of 1.5 μM of sensor was examined with different concentration of NAD⁺. The fluorescence signal of the sensor reduced ˜25% when incubated with 200 μM NAD⁺, and reduced 45% in the presence of 250 μM NAD⁺ (FIG. 14A). This was further confirmed by fluorescence microscopy analysis showing the fluorescence intensity of droplets negatively correlated to the NAD⁺ concentration (FIG. 14B).

### Design and Development of Microfluidic Devices

### Signal Detection of the Droplets

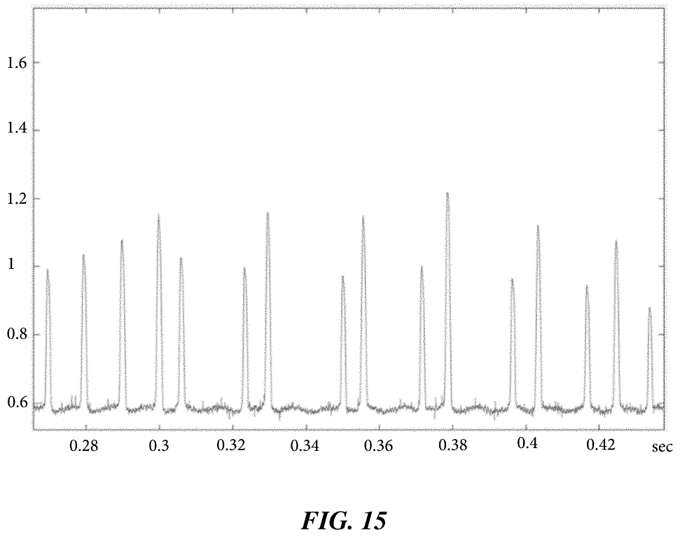

The fluorescence or bioluminescence signals of NAD(P)H-containing droplets was reliably detected with a PMT detector, which outputs a voltage analog signal. An Arduino microcontroller (Due) was used for signal acquisition, A/D conversion and processing. The microcontroller was set to run at free mode that allows 300-500 kHz sample rate with an 8-bits window moving average filter (FIG. 15). This allowed very accurate detection of the change of the signal. The A/D conversion was performed with a 10-bits resolution, which was able to differentiate the fluorescence signal with 0.1% of difference.

The processed signal was then matched against a pre-set sorting threshold. When the signal level reached to a desired range of that threshold, the microcontroller output a digital sorting pulse which enabled sorting action after being amplified.

### Microfluidic Device for Droplet Sorting

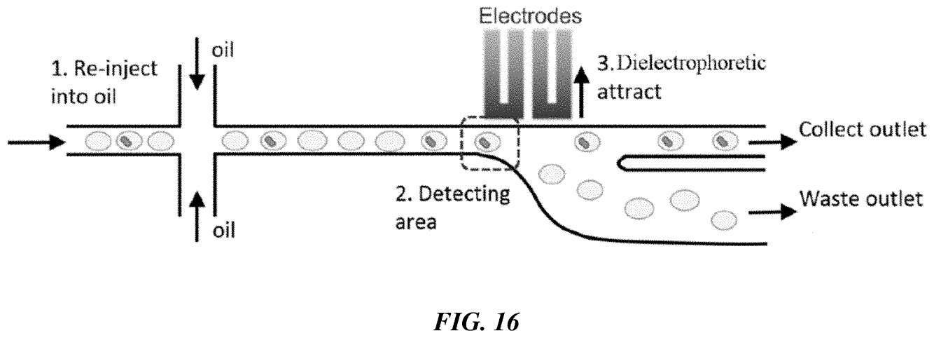

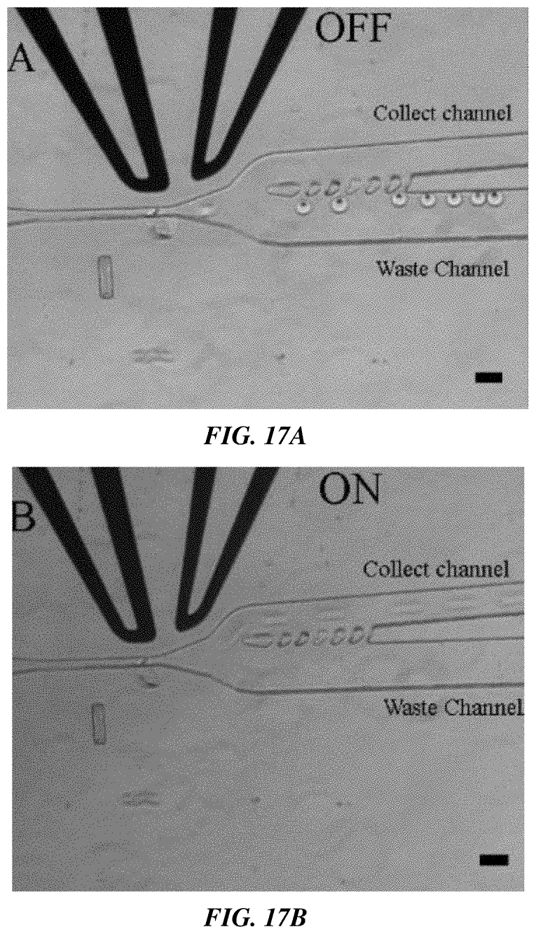

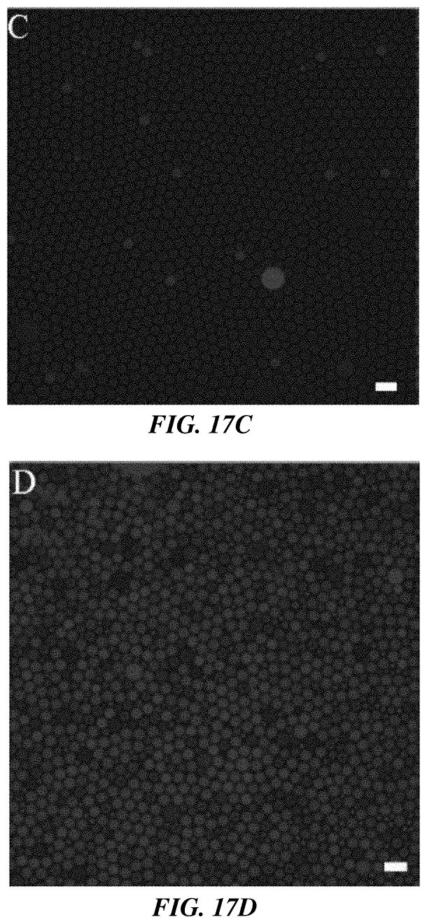

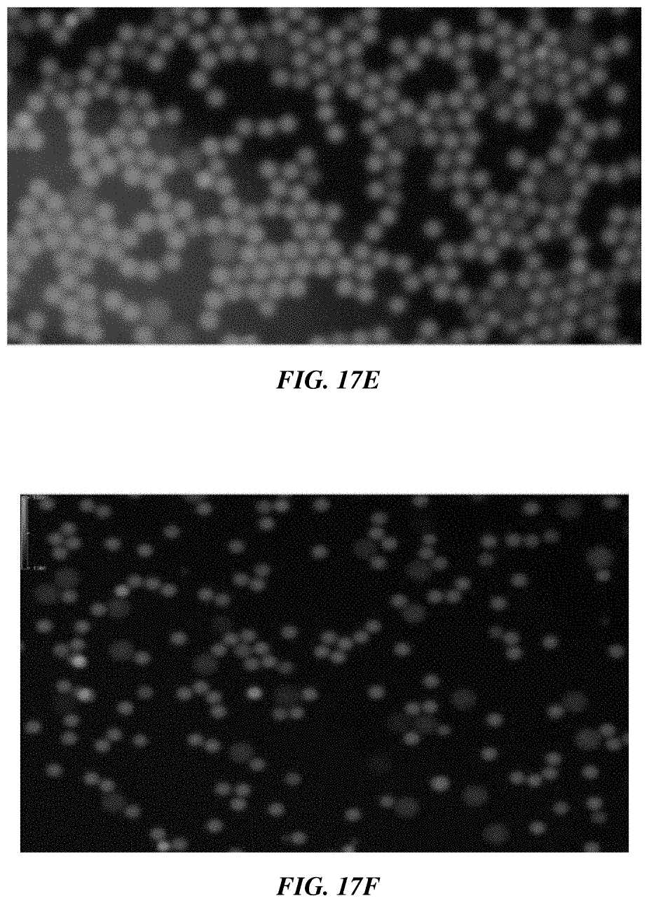

The droplet sorting device is illustrated in FIG. 16. Droplets containing sample of interest were injected into the device consecutively and the inter-droplet space was enlarged by injecting high volume of spacing oil. The fluorescence or bioluminescence signals from each droplet was then detected. Without any sorting action, all droplets flowed to the waste channel (FIG. 17A). Once the signal reached above the desired level, a sorting pulse was triggered. The pulse was amplified to generate a 200-1000 V electric field between sorting electrodes, which pulled the positive droplets away from the path to the waste channel (FIG. 17B).

Coupled with the above-mentioned signal detection hardware, the sorting device was validated through the fluorescence-based sorting of 10% positive fluorescence droplets from 90% of empty (or blank) droplets. After 2 h of sorting, the droplets in the collector contained 92% of positive droplets (FIG. 17D), representing 9-fold sample enrichment. A 95% efficiency was observed as only 0.5% positive droplets remained in the waste channel (FIG. 17C).

Similarly, the sorting of the NADH containing droplets from a mixture of 15% NADH droplets and 85% empty (or blank) droplets was demonstrated (FIG. 17E-17F). The NADH concentration was 10 mM, and the sorting was based on the NADH fluorescence at 450 nm. After 2 h of sorting, the collector channel contained 60% of NADH-containing droplets, showing 4-fold enrichment.

Enrichment of the Droplets Based-On Formed NADH from Reaction of 2-Octanol with Wild-Type CpSADH

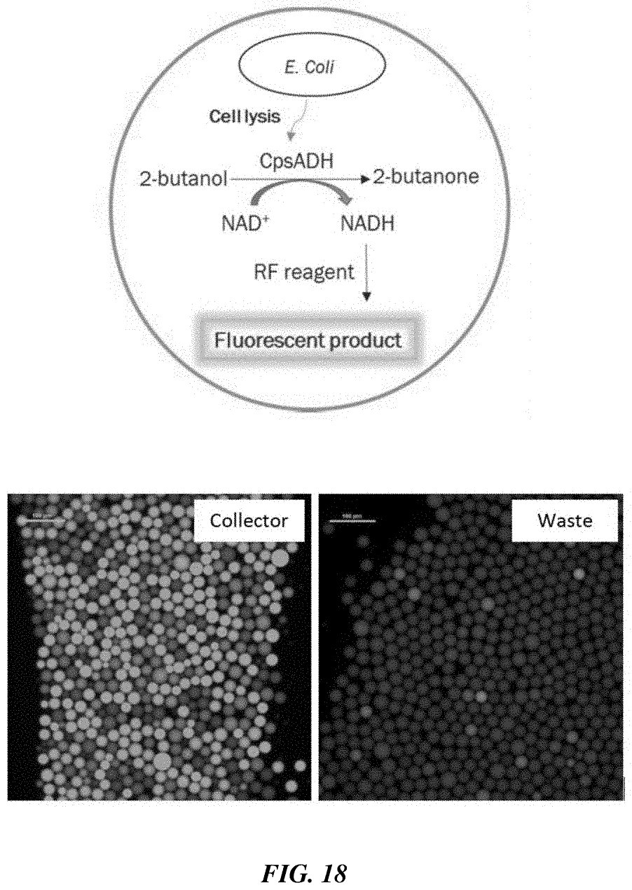

The microfluidic sorting device was validated for the sorting of 10% positive droplets containing single E. coli (CpSADH) cell that catalyzed a NADH-generating reaction and 90% empty (or blank) droplets. In the positive droplets, single cell was lysed to release CpSADH which catalyzed the oxidation of 2-butanol with NAD⁺ to produce stoichiometric amount of NADH. The reagent in the droplet reacted with the NADH to give strong red fluorescence. On the other hand, the empty (or blank) droplets did not produce NADH, thus showing only low background fluorescence. The droplet with high fluorescence were sorted and collected in the collector (FIG. 18). The sorted fractions contained 70% of positive droplets, showing 7-fold enrichment.

The droplet sorting speed could reach to 2000 droplets/sec in the above examples. This high speed sorting could be achieved due to the high sensitivity of the platform of the present invention.

### Droplet Merging Device

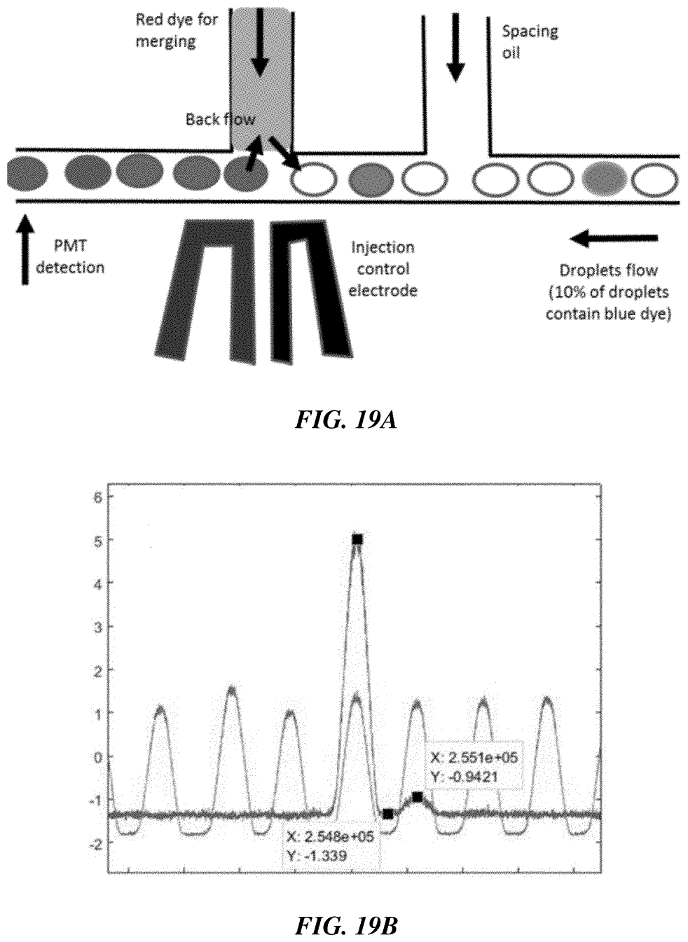

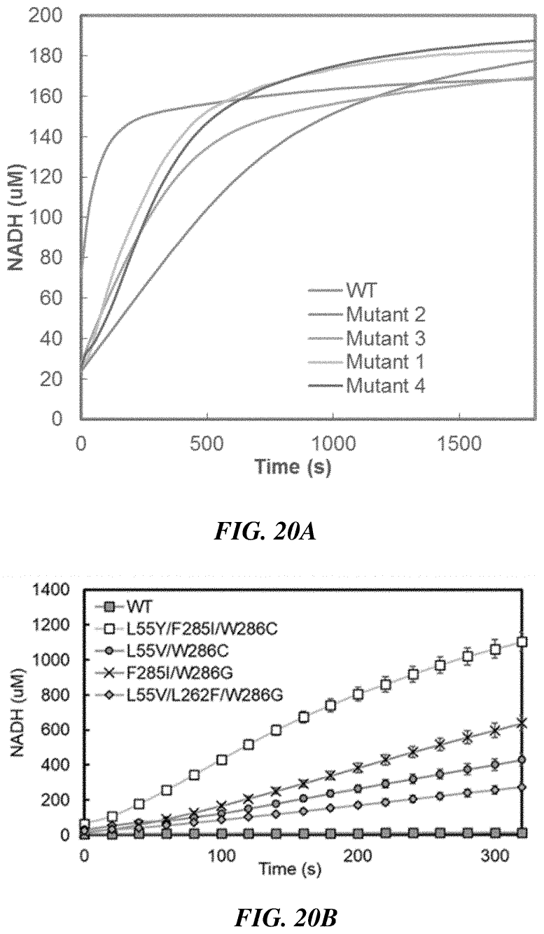

Droplet merging is a technique to reintroduce any reagents into formed droplets, enabling a sophisticated multi-step screening. A merging device was developed to achieve droplet merging as illustrated in FIG. 19A. Droplets were loaded to the device and flushed through the horizontal channel with spacing oil. In the middle of the channel, a perpendicular channel was installed to inject new reagents to the passing droplets through merging. The injection volume and time were precisely controlled by the electrodes.

The merging device was fabricated and tested with a mixture of 90% empty (or blank) droplets and 10% blue fluorescent droplets at a flow rate of 10 droplet/second. A red fluorescent dye was introduced to the droplet mixture through merging at a ratio of 30% volume of the droplets. As shown in the FIG. 19B, the merging process was very consistent with only 5% fluctuation. Examining the blue fluorescence dye in the empty (or blank) droplet that follows immediately the blue droplet shows the sample carry-over (cross-droplet contamination) was less than 7%.

### Directed Evolution of Enzymes Using Microfluidics Screening Platform

The CpSADH was evolved to demonstrate the microfluidics based high-throughput enzyme screening and evolution with an industrially useful enzyme. A targeted library of the CpSADH with 20,000 variants was constructed and transferred into the E. coli, where each cell expresses only a single enzyme variant. The E. coli cells containing variants were grown in 1 mL LB medium containing an appropriate antibiotic at 37° C. After cell OD₆₀₀≃0.6, the IPTG (0.5 mM) was added to induce the expression of enzymes. The cells continued to grow and express enzymes for 12 h at 22° C. They were harvested by centrifugation (4000 g, 10 min), washed twice, and resuspended in an appropriate buffer. A 3-inlet microfluidics chip was used to encapsulate the cells in water microdroplets. A solution containing library cells (OD₆₀₀≃0.02), racemic 2-octanol as substrate, and detection enzyme was injected using a syringe pump from one inlet and the other solution containing red fluorescence reagents, lysis buffer, and NAD⁺ was injected from another inlet and the third inlet was used for oil. The cells then were encapsulated with single cell inside the water-in-oil microdroplets containing the reaction substrate, detection enzyme, and NAD⁺ cofactor. The droplets were sorted according to their red fluorescence signal level that was obtained from the produced NADH. The sorted droplets were subjected to DNA recovery and further analysis of their activity and sequence information. The mutant CpSADH-L55Y/F285I/W286C was identified to provide a 175-folds increase in activity compared to the wild type (WT) for R enantiomer. It also achieved 87.4% of the conversion for racemic mixture while WT could only reach 62%. This new enzyme, unlike the WT, could utilize both enantiomers to avoid wasting the R-enantiomer in conversion of cheaply available racemic 2-octanol to highly desired corresponding ketone.

### EXAMPLE 4

### Example 4

### Directed Evolution and Microfluidic High-Throughput Screening of Secondary Alcohol Dehydrogenase from Candida parapsilosis (CpSADH) as a NAD⁺-Dependent Intracellular Enzyme Using Lysed Cells and NADH Detection

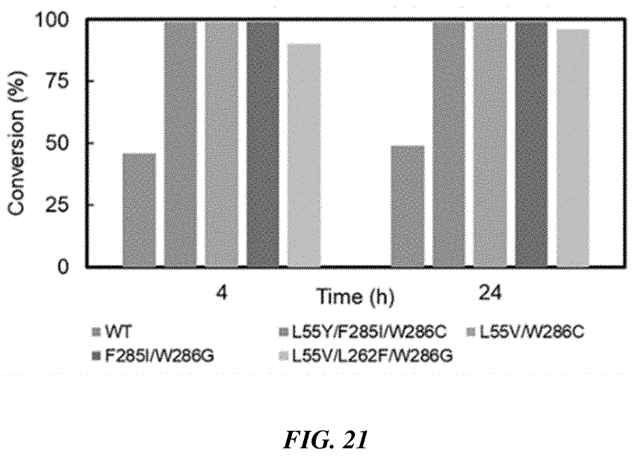

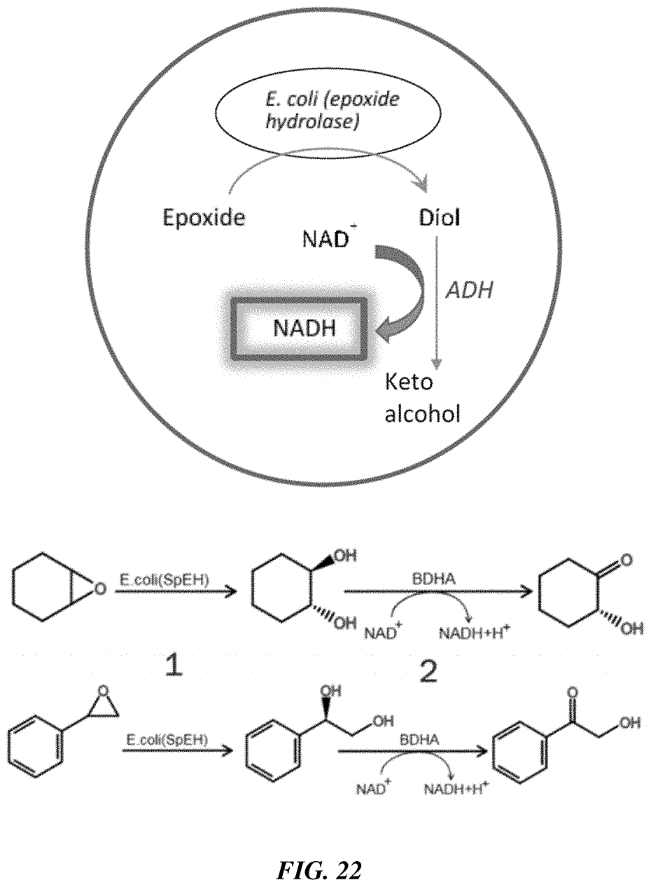

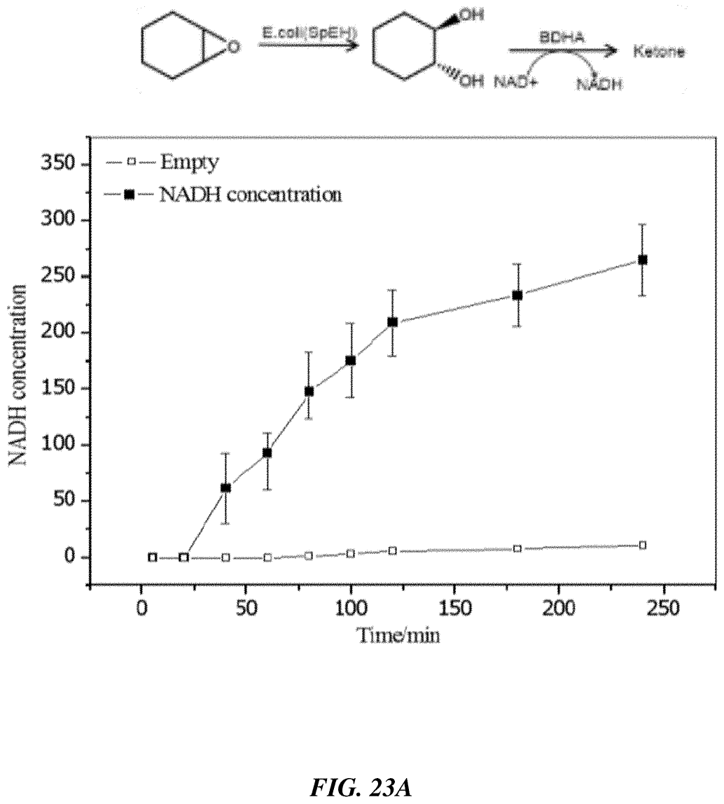

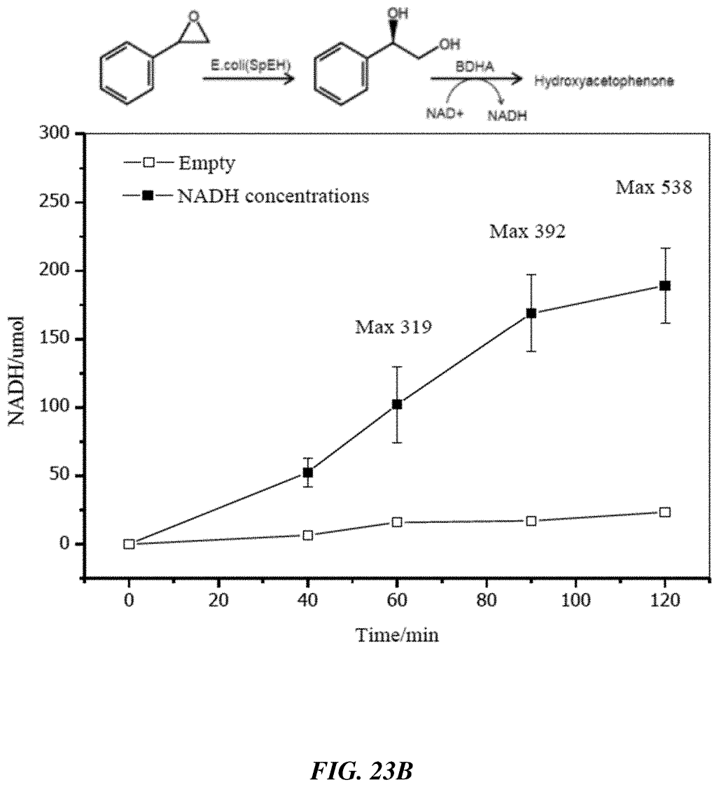

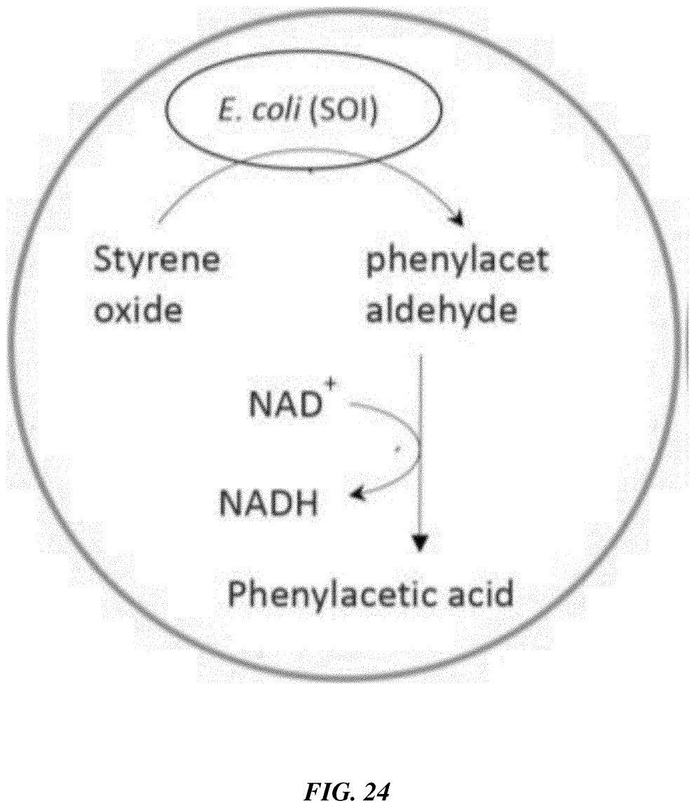

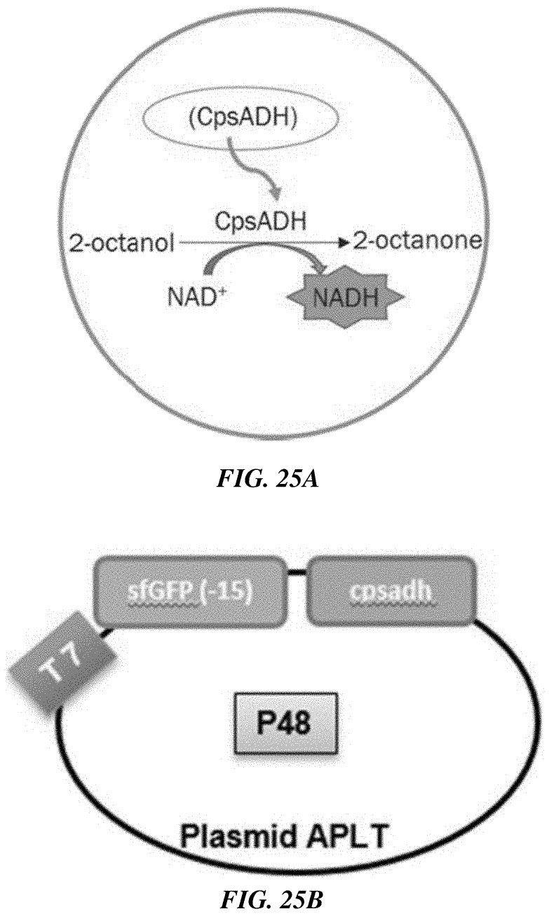

The directed evolution of alcohol dehydrogenase (CpSADH) for non-enantioselective alcohol oxidation was performed. This example demonstrated an example for the system in FIG. 4E with the detection of the produced NADH to sort the droplets. A library of above 20,000 mutants of CpSADH was screened by using microfluidic system. The secondary alcohol dehydrogenase CpSADH is a member of the medium-chain dehydrogenase/reductase (MDR) family of enzymes. CpSADH catalyze reversible redox reaction between alcohol and corresponding ketone with wide substrate scope. Because of its high stereoselectivity, the theoretical conversion is only 50% for the oxidation of racemic alcohol. CpSADH was selected for the engineering to show enhanced R-enantioselectivity or even non-enantioselective for alcohol oxidation. (R)-2-octanol was docked into the active pocket of CpSADH, and four amino acid residues (Leu55, Leu119, Phe285, and Trp286) were selected for simultaneous saturation mutagenesis. NDT degeneracy codon was opted to substitute the selected sites. Then, the mutagenic library was created via overlap extension PCR method. Gene fragments of library was generated by mutagenic primers and flanking primers. The combinatorial mutant library contains 20,736 variants. The E. coli cells containing variants were grown in 1 mL LB medium containing an appropriate antibiotic at 37° C. After cell OD600≃0.6, the IPTG (0.5 mM) was added to induce the expression of enzymes. The cells continued to grow and express enzymes for 12 h at 22° C. They were harvested by centrifugation (4000 g, 10 min) and washed twice and resuspended in an appropriate buffer. A 3-inlet microfluidics chip was used to encapsulate the cells in water microdroplets. A solution containing library cells (OD600≃0.02) and alcohol as substrate was injected using a syringe pump from one inlet and the other solution containing red fluorescence reagents, lysis buffer, and NAD⁺ was injected from another inlet and the third inlet was used for oil. The cells of the library were encapsulated with single cell inside the water-in-oil microdroplets containing the reaction substrate, lysis buffer, and NAD^{≃} cofactor. After incubation, droplets were screened and those with fluorescence intensity higher than the specified threshold were identified as possible hits and thus sorted. The collected droplets were subjected to post-sorting analysis to get the enzyme sequence. A group of enzyme mutants were identified (Table 2), including the four variants Mutant1-Mutant4 (CpSADH-L55V/L262F/W286G, CpSADH-L55Y/F285I/W286C, CpSADH-L55V/W286C, and CpSADH-F285I/W286G, respectively) showing higher activity to (R)-2-butanol than wild type were obtained. The kcat/Km of the Mutant2 showed 175-folds increase compare to the WT for R enantiomer (Table 3). Further characterization of the mutants showed that the full conversion of the racemic 2-octanol to 2-octanone using NADH regeneration system while WT could reach up to 49% conversion at the same reaction conditions (FIG. 21).

TABLE 2
The best performing mutants of CpSADH for the oxidation of racemic
2-octanol obtained from microfluidics-based screening.

[symbol]

[symbol]

indicates data missing or illegible when filed

TABLE 3
Catalytic kinetic constant of the wild type (WT) and mutants of
CpSADH for S- and R-2-octanol enantiomers.

[symbol]

[symbol]

indicates data missing or illegible when filed

TABLE 4
Conversion of the oxidation of 2-octanol with
different CpsADH variants.

| Time | | | | | |
|---|---|---|---|---|---|
| (h) | WT | Mutant 1 | Mutant 2 | Mutant 3 | Mutant 4 |
| 1 | 51.3% | 63.6% | 66.4% | 61.8% | 63.2% |
| 2 | 53.8% | 70.8% | 71.1% | 67.1% | 68.1% |
| 3 | 58.6% | 71.3% | 73.5% | 71.4% | 70.5% |
| 4 | 61.4% | 72.4% | 73.7% | 77.7% | 73.7% |
| 12 | 62.4% | 77.1% | 87.4% | 85.7% | 79.0% |

### EXAMPLE 5

### Example 5

### Directed Evolution and Microfluidic High-Throughput Screening of Epoxide Hydrolase from Sphingomonas sp. HXN-200 (SpEH) as a Cofactor-Independent Intracellular Enzyme Using Whole-Cells and NADH Detection via Additional Enzymatic Reaction of the Target Product

This example demonstrated screening and evolution of epoxide hydrolase (EH) with a procedure as described in FIG. 4A. A mutant library of the epoxide hydrolase from Sphingornonas sp. HXN-200 (SpEH) with 10⁴-10⁷ variants was prepared and transferred into the host microorganism (E. coli) where each cell expressed only a single enzyme variant. The E. coli cells containing variants were grown in 1 mL LB medium containing an appropriate antibiotic at 37° C. After cell OD600≃0.6, the IPTG (0.5 mM) was added to induce the expression of enzymes. The cells continued to grow and express enzymes for 12 h at 22° C. They were harvested by centrifugation (4000 g, 10 min) and washed twice and resuspended in an appropriate buffer. A 3-inlet microfluidics chip was used to encapsulate the cells in water microdroplets. A solution containing library cells (OD600≃0.02), epoxide as substrate, and detection enzyme (an ADH) was injected using a syringe pump from one inlet and the other solution containing red fluorescence reagents and NAD⁺ was injected from another inlet and the third inlet was used for oil. The cells then were encapsulated with single cell inside the water-in-oil microdroplets containing the epoxide, detection enzyme, and NAD⁺ cofactor. The droplets were sorted according to the red fluorescence signal that was obtained from the produced NADH via cascade reaction of diol to keto alcohol. The sorted cells were subjected to further analysis and plated on LB-Agar plate containing an appropriate antibiotic to analyze their activity and obtain their sequence information.

### EXAMPLE 6

### Example 7

### Microfluidic High-Throughput Screening for Directed Evolution of Styrene Oxide Isomerase (SOI) as a Cofactor-Independent Intracellular Enzyme Using Whole-Cells and NADH Detection via Additional Enzymatic Reaction of the Target Product

This example demonstrated screening and evolution of styrene oxide isomerase (SOI) with a procedure as described in FIG. 4A. A mutant library of the SOI (10⁴-10⁷ variants) was prepared and transferred into the host microorganism (E. coli), where each cell expresses only a single enzyme variant. The E. coli cells containing variants were grown in 1 mL LB medium containing an appropriate antibiotic at 37° C. After cell OD600≃0.6, the IPTG (0.5 mM) was added to induce the expression of enzymes. The cells continued to grow and express enzymes for 12 h at 22° C. They were harvested by centrifugation (4000 g, 10 min) and washed twice and resuspended in an appropriate buffer. A 3-inlet microfluidics chip was used to encapsulate the cells in water microdroplets. A solution containing library cells (OD600≃0.02), styrene oxide as substrate, and detection enzyme was injected using a syringe pump from one inlet and the other solution containing red fluorescence reagents and NAD⁺ was injected from another inlet and the third inlet was used for oil. The cells then were encapsulated with single cell inside the water-in-oil microdroplets containing the reaction substrate, detection enzyme, and NAD⁺ cofactor. The droplets were sorted according to the red fluorescence signal that was obtained from the produced NADH via cascade reaction of phenylacetaldehyde to phenylacetic acid. The sorted cells were subjected to further analysis and plated on LB-Agar plate containing an appropriate antibiotic to analyze their activity and obtain their sequence information.

### EXAMPLE 7

### Example 7

### Microfluidic High-Throughput Screening for Directed Evolution of Secondary Alcohol Dehydrogenase from Candida parapsilosis (CpSADH) as a NAD⁺-Dependent Surface Displayed Enzyme Using NADH Detection

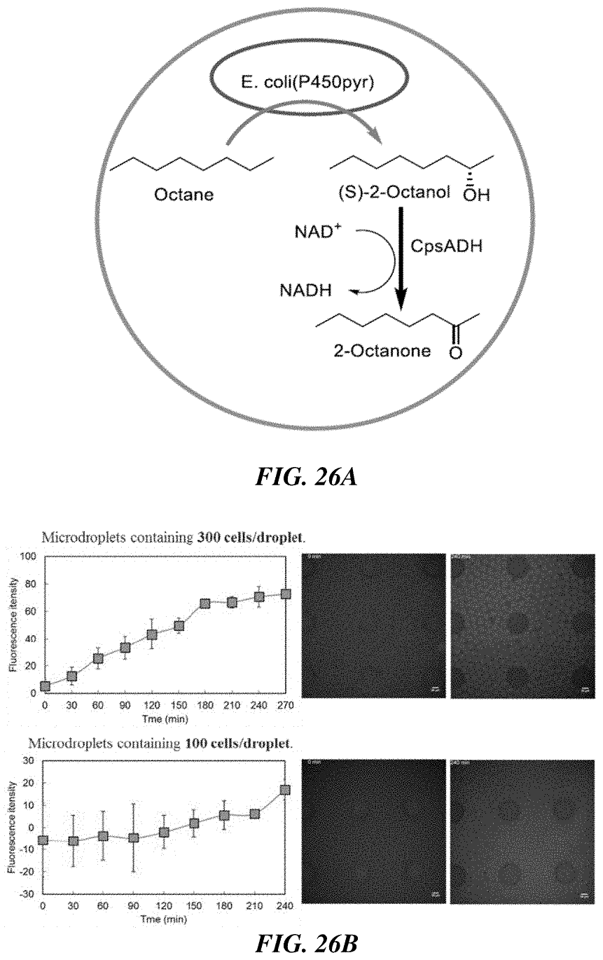

This example demonstrated screening and evolution of alcohol dehydrogenase (CpSADH) as an NADH-dependent enzyme that is surface displayed with E. coli. The plasmid is constructed as illustrated in FIG. 26. The screening procedure was as described in FIG. 4D. A mutant library of the CpSADH (10⁴-10⁷ variants) was prepared and transferred into the host microorganism (E. coli), where each cell expressed only a single enzyme variant. The E. coli cells containing variants were grown in 1 mL LB medium containing an appropriate antibiotic at 37° C. After cell OD600≃0.6, the IPTG (0.5 mM) was added to induce the expression of enzymes. The cells continued to grow and express enzymes for 12 h at 22° C. They were harvested by centrifugation (4000 g, 10 min) and washed twice and resuspended in an appropriate buffer. A 3-inlet microfluidics chip was used to encapsulate the cells in water microdroplets. A solution containing library cells (OD600≃0.02) and 2-octanol as substrate was injected using a syringe pump from one inlet and the other solution containing red fluorescence reagents and NAD⁺ was injected from another inlet and the third inlet was used for oil. The cells of the library are encapsulated with single cell inside the water-in-oil microdroplets containing the reaction substrate, red fluorescence reagent, and NAD⁺ cofactor. The 2-octanol substrate is converted to ketone and the reaction was detected via sensing the produced NADH. The droplets were sorted according to the red fluorescence signal that is obtained from the produced NADH. The sorted cells were subjected to further analysis and plated in LB-Agar plate containing an appropriate antibiotic to analyze their activity and obtain their sequence information.

### EXAMPLE 8

### Example 8

### Microfluidic High-Throughput Screening for Directed Evolution of Epoxide Hydrolase from Sphingomonas sp. HXN-200 (SpEH) as Cofactor-Independent Intracellular Enzyme with Lysed Cells and NADH Detection via Additional Enzymatic Reaction of the Target Product

To demonstrate an example for this category, the SpEH as cofactor independent enzyme was used and the procedure was as described in FIG. 4C. A mutant library of the SpEH (10⁴-10⁷ variants) was prepared and transferred into the host microorganism (E. coli), where each cell expressed only a single enzyme variant. The E. coli cells containing variants were grown in 1 mL LB medium containing an appropriate antibiotic at 37° C. After cell OD600≃0.6, the IPTG (0.5 mM) was added to induce the expression of enzymes. The cells continued to grow and express enzymes for 12 h at 22° C. They were harvested by centrifugation (4000 g, 10 min) and washed twice and resuspended in an appropriate buffer. A 3-inlet microfluidics chip was used to encapsulate the cells in water microdroplets. A solution containing library cells (OD600≃0.02) and epoxide as substrate was injected using a syringe pump from one inlet and the other solution containing red fluorescence reagents, lysis buffer, and NAD⁺ was injected from another inlet and the third inlet was used for oil. The cells of the library were encapsulated with single cell inside the water-in-oil microdroplets containing the reaction substrate, detection enzyme, lysis buffer, and NAD⁺ cofactor. The droplets are sorted according to the fluorescence signal that is obtained from the produced NADH using diaphorase enzyme and resazurin. The droplets were sorted according to the red fluorescence signal that was obtained from the produced NADH via cascade reaction of diol to keto alcohol. The sorted droplets were subjected to further analysis to recover the DNA from the droplets. The obtained DNA was then transformed into electrocompetent E. coli cells (obtained from Lucigen) to analyze their activity and sequence information.

### EXAMPLE 9

### Example 9

### Microfluidic High-Throughput Screening for Directed Evolution of P450 Monooxygenase (P450pyr) as a Cofactor-Dependent Intracellular Enzyme Through Cell-Growth and Droplet-Merging Using Whole-Cells with NADH Detection via Additional Enzymatic Reaction of the Target Product

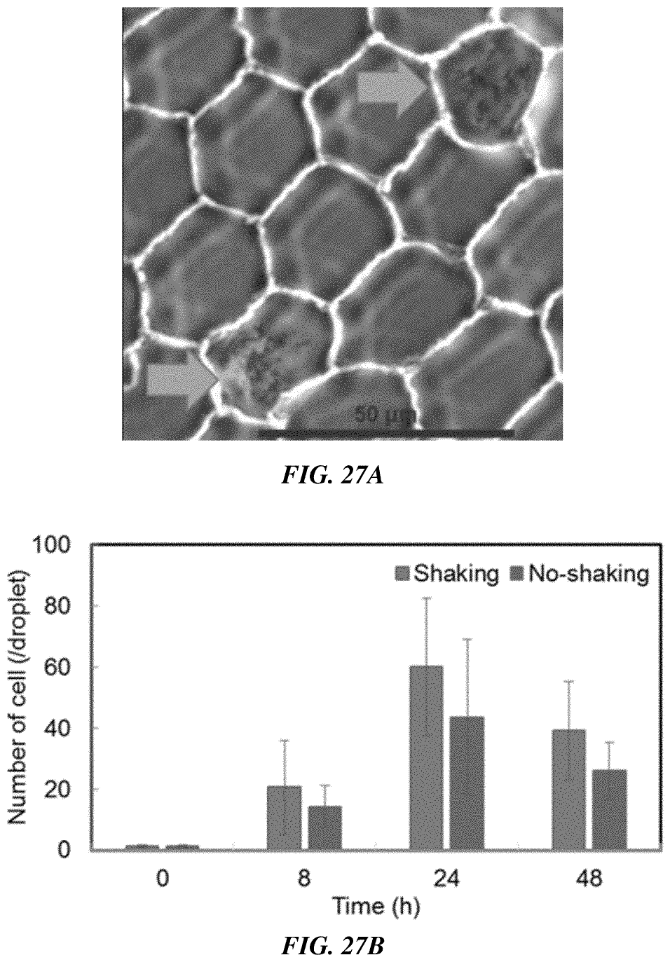

This example demonstrated screening and evolution of P450pyr with a procedure as described in FIG. 5A. A mutant library of the P450pyr (10⁴-10⁷ variants) was prepared and transferred into the host microorganism (E. coli), where each cell expressed only a single enzyme variant. The cells expressing variants were grown in 1 mL LB medium containing an appropriate antibiotic at 37° C. They were harvested by centrifugation (4000 g, 10 min) and washed twice and resuspended in culture media. A 2-inlet microfluidics chip was used to encapsulate the cells in microdroplets. A solution containing library cells (OD600≃0.02) and appropriate medium was injected from one inlet and oil from another using a syringe pump to achieve single-cell encapsulation. The cells were then allowed to grow through the incubation at 30-37° C. and shaking conditions. The single cell inside a droplet could reproduce to increase the number of cells (FIG. 27). The merging system was used to add inducer, octane as substrate, detection enzyme (an ADH), red fluorescence reagents, and NAD⁺ into the droplets. The octane converted to octanol and it was further converted to ketone through a cascade using detection enzyme (ADH). The produced NADH via cascade reaction was detected with red fluorescence reagent and the droplets were sorted. The sorted cells were subjected to further analysis and plated in LB-Agar plate containing an appropriate antibiotic to analyze their activity and obtain their sequence information.

### EXAMPLE 10

### Example 10

### Microfluidic High-Throughput Screening for Directed Evolution of Amine Dehydrogenase from Rhodococcus (AmDH) as a NAD⁺-Dependent Intracellular Enzyme Through Cell-Growth and Droplet-Merging Using Lysed Cells with NADH Detection

Amine dehydrogenase (AmDH) is a useful enzyme for enantionioselective amination. The triple mutant AmDH showed a low catalytic activity. As AmDH is a cofactor dependent enzyme, the produced NADH in the deamination reaction would be detected. Initial efforts to directly lye the cells and perform the reaction with single-cell showed poor sorting enrichment. Therefore, to detect and sort the AmDH variants in microfluidics system, the cells needed to be grown in the droplet then followed by droplet-merging to add reaction reagents and lysis buffer into the droplets. The procedure was as described in FIG. 5E. A mutant library of the AmDH (10⁴-10⁷ variants) was prepared and transferred into the host microorganism (E. coli), where each cell expressed only a single enzyme variant. The cells containing variants were grown in 1 mL LB medium containing an appropriate antibiotic at 37° C. They were harvested by centrifugation (4000 g, 10 min) and washed twice and resuspended in culture media. A 2-inlet microfluidics chip was used to encapsulate the cells in microdroplets. A solution containing library cells (OD600≃0.02) and appropriate medium was injected from one inlet and oil from another using a syringe pump to achieve single-cell encapsulation. The cells were then allowed to grow through the incubation at 30-37° C. and shaking conditions. The single cell inside a droplet could reproduce to increase the number of cells (FIG. 27). The merging system was used to add inducer, lysis buffer, amine as substrate, red fluorescence reagents, and NAD⁺ into the droplets. The amine is converted to ketone and the produced NADH was detected using red fluorescence reagent. The droplets were sorted according to the fluorescence signal. The sorted droplets were subjected to further analysis to recover the DNA from the droplets. The obtained DNA was then transformed into electrocompetent E. coli cells (obtained from Lucigen) to analyze their activity and sequence information.

### EXAMPLE 11

### Example 11

### Microfluidic High-Throughput Screening for Directed Evolution of Secondary Alcohol Dehydrogenase from Candida parapsilosis (CpSADH) as a NADH-Dependent Extracellular Enzyme Through Cell-Growth and Droplet-Merging with NAD⁺ Detection Using Biosensor or Quantum Dots

This example demonstrated screening and evolution of CpSADH with a procedure as described in FIG. 5D. NAD⁺ cofactor detection using biosensor was demonstrated. A mutant library of the CpsADH (10⁴-10⁷ variants) is prepared and transferred into the host microorganism (E. coli) for extracellular expression, where each cell expressed only a single enzyme variant. The cells containing variants were grown in 1 mL LB medium containing an appropriate antibiotic at 37° C. They were harvested by centrifugation (4000 g, 10 min) and washed twice and resuspended in culture media. A 2-inlet microfluidics chip was used to encapsulate the cells in microdroplets. A solution containing library cells (OD600≃0.02) and appropriate medium was injected from one inlet and oil from another using a syringe pump to achieve single-cell encapsulation. The cells were then allowed to grow through the incubation at 30-37° C. and shaking conditions. The single cell inside a droplet could reproduce to increase the number of cells. The merging system was used to add inducer, butanone as non-natural substrate, NAD⁺ biosensor or quantum dots, and NADH into the droplets. The freshly expressed extracellular enzymes performed the conversion of butanone to butanol. The produced NAD⁺ was detected via its interaction with biosensor and the obtained signal was used for droplet sorting. The sorted cells were subjected to further analysis and plated in LB-Agar plate containing an appropriate antibiotic to analyze their activity and obtain their sequence information.

### EXAMPLE 12

### Example 12

### Microfluidic High-Throughput Screening for Directed Evolution of Styrene Oxide Isomerase (SOI) as Cofactor-Independent Intracellular Enzyme Through Cell-Growth and Droplet-Merging Using Lysed Cells and NADH Detection via Additional Enzymatic Reaction of the Target Product

This example demonstrated screening and evolution of styrene oxide isomerase (SOI) with a procedure as described in FIG. 5C. In this example, SOI as non-NADH cofactor dependent was evolved to accept aliphatic epoxide as non-natural substrate. A mutant library of the SOI (10⁴-10⁷ variants) was prepared and transferred into the host microorganism (E. coli), where each cell expressesonly a single enzyme variant. The E. coli cells containing variants were grown in 1 mL LB medium containing an appropriate antibiotic at 37° C. After cell OD600≃0.6, the IPTG (0.5 mM) was added to induce the expression of enzymes. The cells continued to grow and express enzymes for 12 h at 22° C. They were harvested by centrifugation (4000 g, 10 min) and washed twice and resuspended in an appropriate buffer. A 2-inlet microfluidics chip was used to encapsulate the cells in microdroplets. A solution containing library cells (OD600≃0.02) and appropriate medium was injected from one inlet and oil from another using a syringe pump to achieve single-cell encapsulation. The cells were then allowed to grow through the incubation at 30-37° C. and shaking conditions. The single cell inside a droplet could reproduce to increase the number of cells. The merging system was used to add inducer, butanone as non-natural substrate, detection enzyme, and NAD⁺ into the droplets. The cells then were encapsulated with single cell inside the water-in-oil microdroplets containing the reaction substrate, lysis buffer, detection enzyme, and NAD⁺ cofactor. The cells were lysed and aliphatic epoxide then converted to the corresponding aldehyde. The droplets were sorted according to the red fluorescence signal that was obtained from the produced NADH via cascade reaction of aldehyde to acetic acid. The sorted droplets were subjected to further analysis to recover the DNA from the droplets. The obtained DNA was then transformed into electrocompetent E. coli cells (obtained from Lucigen) to analyze their activity and sequence information.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of screening an enzyme for one or more characteristics, the method comprising the steps of:
(a) generating a plurality of droplets, wherein at least one droplet comprises:
an enzyme, a reaction substrate; and one or more redox cofactors, or one or more redox cofactors and one or more detection reagents, wherein the enzyme converts the reaction substrate to a target product;
(b) detecting a signal emitted from the droplet, wherein the signal is a fluorescence signal, a bioluminescence signal or a chemiluminescence signal, (i) wherein the signal is emitted when the redox cofactor is oxidized or reduced when the reaction substrate is converted to the target product by the enzyme, or (ii) wherein the signal is emitted when the redox cofactor is oxidized or reduced when the target product is further converted to a subsequent product by the one or more detection reagents in the droplet.

2. A method of screening an enzyme for one or more characteristics, comprising:
in a fluidic device comprising an incubation region comprising a plurality of incubation chambers including a first incubation chamber and a second incubation chamber, wherein the plurality of incubation chambers are configured to allow continuous flow of a plurality of droplets, performing the steps of:
culturing a cell in the droplet to form a plurality of cells while flowing the cell from the first incubation chamber to the second incubation chamber;
continuously flowing the droplet in the incubation region, wherein the droplet comprises, an enzyme, a reaction substrate, and one or more redox cofactors, or one or more redox cofactors and one or more detection reagents, wherein the enzyme converts the reaction substrate to a target product; and
generating a signal from the droplet, wherein the signal is a fluorescence signal, a bioluminescence signal or a chemiluminescence signal.

3. (canceled)

4. A fluidic system, comprising:
a microfluidic channel containing a plurality of droplets,
wherein at least one droplet comprises a redox cofactor and a detection reagent; and
wherein the detection reagent is configured to react with a redox couple of the redox cofactor to produce a luminescent signal.

5-8. (canceled)

9. The method of claim 1, further comprising screening the enzyme for the one or more characteristics based on the level of emitted signal detected compared to a reference signal, wherein a change in the level of emitted signal compared to the reference signal indicates that the enzyme has the one or more characteristics.

10. The method of claim 1, further comprising isolating the droplets based on the level of emitted signal detected compared to a reference signal.

11. (canceled)

12. The method of claim 1, further comprising culturing cells within the plurality of droplets.

13. (canceled)

14. The method of claim 1, further comprising lysing one or more cells.

15. The method of claim 1, wherein detecting comprises detecting intracellular or cell surface-displayed enzymes.

16. The method of claim 1, wherein detecting comprises detecting extracellular enzymes.

17-18. (canceled)

19. The method of claim 1, wherein the enzyme is a redox cofactor-dependent enzyme and the signal is emitted when the redox cofactor is oxidized or reduced when the reaction substrate is converted to the target product by the enzyme.

20. The method of claim 1, wherein the enzyme is a redox cofactor-independent enzyme and the signal is emitted when the redox cofactor is oxidized or reduced when the target product is further converted to a subsequent product by the one or more detection reagents.

21. The method of claim 1, wherein generation of the plurality of droplets in step a) further comprises incubating the host cell in each droplet with culture media in the droplet to increase the number of host cells prior to adding the reaction substrate, detection reagents, one or more detection reagents, lysis buffer, redox cofactor or combinations thereof.

22. The method of claim 1, wherein the droplets are isolated by using a method selected from the group consisting of fluorescence-activated droplet sorting (FADS), fluorescence-activated cell sorting (FACS) and magnetic-activated cell sorting (MACS), acoustic control techniques, magnetic control techniques, pneumatic control techniques, thermal control techniques, electric control techniques and combinations thereof.

23. The method of claim 1, wherein the one or more characteristics of the enzyme is selected from the group consisting of enzyme activity, stability, specificity, stereoselectivity, ability to convert a non-natural substrate and combinations thereof.

24. The method of claim 1, further comprising the step of sequencing an amino acid sequence, a polynucleotide sequence, or both, of the enzyme in the isolated droplets, optionally wherein the sequenced enzyme is used to generate a library of enzyme variants.

25. The method of claim 1, wherein the enzyme is expressed in a host cell, and wherein the plurality of droplets further comprises the host cell or fragments thereof.

26-30. (canceled)

31. The method of claim 1, wherein the one or more redox cofactors is selected from the group consisting of NAD+, NADH, NADP+, NADPH, NAD+ and NADP+, and NADH and NADPH.

32. The method of claim 1, wherein each droplet of the plurality of droplets further comprises one or more detection reagents that react with the oxidized or reduced redox cofactor to produce the emitted signal.

33-41. (canceled)

42. The method of claim 2, wherein the cell is selected from the group consisting of a mammalian cell, a plant cell, a bacterial cell, a fungal cell, a yeast cell, a protozoan cell, an algal cell and an archaeal cell.

43-48. (canceled)

49. The method of claim 1, wherein the enzyme is an enzyme variant selected from a library of enzyme variants of a single enzyme, or wherein the enzyme is selected from a group of different enzymes.

50-53. (canceled)
